(19) United States
(12) Patent Application Publication
(10) Pub. No.: US 2011/0030077 A1
(43) Pub. Date: Feb. 3, 2011

(54) METHODS OF MODULATING THE ORGANIC SOLUTE AND STEROID TRANSPORTER (OSTalpha-OSTbeta) ACTIVITY AND TREATING ASSOCIATED CONDITIONS

(75) Inventors: Nazzareno Ballatori, Rochester, NY (US); Na Li, Rochester, NY (US)

Correspondence Address: NIXON PEABODY LLP - PATENT GROUP, 1100 CLINTON SQUARE, ROCHESTER, NY, 14604, US

(73) Assignee: UNIVERSITY OF ROCHESTER, Rochester, NY (US)

(21) Appl. No.: 12/865,579

(22) PCT Filed: Feb. 2, 2009
(86) PCT No.: PCT/US09/32872

§ 371 Date: Sep. 8, 2010

(51) Int. Cl. A01K 67/027; A61K 39/395; A61K 38/16; A61P 3/06; A61K 31/7088; C07K 14/00

(52) U.S. Cl. 800/14; 424/139.1; 514/7.4; 514/44 R; 536/23.1; 530/300

(57) ABSTRACT

The present invention is directed to methods of modulating bile acid and lipid transport via the organic solute and steroid transporter Ostα-Ostβ. The present invention is further directed to methods of treating a patient having dyslipidemia or a condition associated with altered bile acid homeostasis. Therapeutic agents and pharmaceutical compositions that modulate Ostα-Ostβ heteromeric complex formation and/or transport activity are also disclosed.

This application claims the benefit of U.S. Provisional Patent Application Ser. Nos. 61/025,690 filed Feb. 1, 2008, and 61/035,226 filed Mar. 10, 2008, each of which is hereby incorporated by reference in its entirety.

This invention was made with government support under grant numbers DK067214 and DK48823 awarded by the NIH. The government has certain rights in this invention.

### FIELD OF THE INVENTION

The present invention is directed to methods and therapeutic agents useful for modulating transport activity of the organic solute and steroid transporter, Ostα-Ostβ. Treatment of various conditions associated with Ostα-Ostβ activity or inactivity are also disclosed.

### BACKGROUND OF THE INVENTION

The steroid-derived class of compounds, including bile acids, steroid hormones, and other cholesterol metabolites, play critical roles in human physiology; however, relatively little is known about the transport proteins that mediate cellular import and export of these molecules. Of relevance, recent studies have identified a plasma membrane transporter, the heteromeric Ostα-Ostβ, which plays a critical role in the transport of bile acids and conjugated steroids in the body (Wang et al., “Expression Cloning of Two Genes that Together Mediate Organic Solute and Steroid Transport in the Liver of a Marine Vertebrate,” Proc. Natl. Acad. Sci. U.S.A. 98:9431-9436 (2001); Seward et al., “Functional Complementation Between a Novel Mammalian Polygenic Transport Complex and an Evolutionarily Ancient Organic Solute Transporter, Ostα-Ostβ,” J. Biol. Chem. 278:27473-27482 (2003); Dawson et al., “The Heteromeric Organic Solute Transporter α-β, Ostα-Ostβ, is an Ileal Basolateral Bile Acid Transporter,” J. Biol. Chem. 280:6960-6968 (2005); Ballatori et al., “Ostα-Ostβ, a Major Basolateral Bile Acid and Steroid Transporter in Human Intestinal, Renal, and Biliary Epithelia,”Hepatology 42:1270-1279 (2005); Boyer et al., “Up-Regulation of a Basolateral Fxr-Dependent Bile Acid Efflux Transporter, Ostα-Ostβ, in Cholestasis in Humans and Rodents,” Am. J. Physiol. 290:G1124-G1130 (2006); Li et al., “Heterodimerization, Trafficking, and Membrane Topology of the Two Proteins, Ostα and Ostβ, that Constitute the Organic Solute and Steroid Transporter,” Biochem. J. 407:363-372 (2007); Hwang et al., “Arachidonic Acid-Induced Expression of the Organic Solute and Steroid Transporter-Beta, Ostβ, in a Cartilaginous Fish Cell Line,”Comparative Biochem. Physiol. 148:39-47 (2008); and Ballatori et al., “Ostα-Ostβ is Required for Bile Acid and Conjugated Steroid Disposition in the Intestine, Kidney, and Liver,” Am. J. Physiol. 295:G179-G186 (2008)).

Bile acids are the major products of cholesterol catabolism, and they regulate a multitude of biological processes, including hepatic bile secretion and the intestinal absorption of fat and fat-soluble vitamins (A. F. Hofmann, “Bile Acids, Cholesterol, Gallstone Calcification, and the Enterohepatic Circulation of Bilirubin,” Gastroenterology 116:1276-1277 (1999); Trauner et al., “Bile Salt Transporters: Molecular Characterization, Function, and Regulation,” Physiol. Rev. 83:633-671 (2003); and A. F. Hofmann, “Biliary Secretion and Excretion in Health and Disease: Current Concepts,” Ann. Hepatol. 6:15-27 (2007)). Bile acids also modulate triglyceride, cholesterol, energy, and glucose homeostasis through their activation of specific receptors and signaling pathways (Houten et al., “Endocrine Functions of Bile Acids,”EMBO J. 25:1419-1425 (2006)); regulate pancreatic secretions and the release of gastrointestinal peptides (Koop et al., “Physiological Control of Cholecystokinin Release and Pancreatic Enzyme Secretion By Intraduodenal Bile Acids,” Gut 39:661-667 (1996) and Riepl et al., “Effect of Intraduodenal Taurodeoxycholate and L-Phenylalanine on Pancreatic Secretion and on Gastroenteropancreatic Peptide Release in Man,” Eur. J. Med. Res. 1:499-505 (1996)); activate carboxyl ester lipase (Kirby et al., “Bile Salt-Stimulated Carboxyl Ester Lipase Influences Lipoprotein Assembly and Secretion in Intestine: A Process Mediated Via Ceramide Hydrolysis,” J. Biol. Chem. 277:4104-4109 (2002)); promote the propulsive motility of the muscle layer in colon (Flynn et al., “The Effect of Bile Acids on Colonic Myoelectrical Activity,” Br. J. Surg. 66:776-779 (1979) and Shiff et al., “Mechanism of Deoxycholic Acid Stimulation of the Rabbit Colon,” J. Clin. Invest. 69:985-992 (1982)); induce bile flow and biliary lipid secretion in the liver, and promote mitosis during hepatic regeneration (A. F. Hofmann, “Biliary Secretion and Excretion in Health and Disease: Current Concepts,” Ann. Hepatol. 6:15-27 (2007) and Geier et al., “Bile Acids are “Homeotrophic” Sensors of the Functional Hepatic Capacity and Regulate Adaptive Growth During Liver Regeneration,” Hepatology 45:251-253 (2007)); solubilize cholesterol, trap cholephilic xenobiotics in micelles, and stimulate bicarbonate secretion via cystic fibrosis transmembrane conductance regulator (CFTR) and anion exchanger AE2 in the biliary tract, as well as promote proliferation when bile duct secretion is obstructed (Alpini et al., “Bile Acids Stimulate Proliferative and Secretory Events in Large But Not Small Cholangiocytes,” Am. J. Physiol. 273:G518-G529 (1997) and Dray-Charier et al., “Regulation of Mucin Secretion in Human Gallbladder Epithelial Cells: Predominant Role of Calcium and Protein Kinase C,” Gastroenterology 112:978-990 (1997)); stimulate thermogenesis by thyroid hormone in brown adipose tissue (Watanabe et al., “Bile Acids Induce Energy Expenditure by Promoting Intracellular Thyroid Hormone Activation,” Nature 439:484-489 (2006)); and play a role in regulating gene expression via nuclear receptors and triggering various signal pathways impinging on numerous cellular processes, including apoptosis (Jones et al., “Bile Salt-Induced Apoptosis of Hepatocytes Involves Activation of Protein Kinase C,” Am. J. Physiol. 272:G1109-G1115 (1997) and Kullak-Ublick et al., “Enterohepatic Bile Salt Transporters in Normal Physiology and Liver Disease,” Gastroenterology 126:322-342 (2004)).

Bile acids are synthesized in the liver and are delivered via the bile into the small intestine, where they aid in the solubilization of dietary lipids (especially fats and lipid soluble vitamins), lipophilic drugs, and electrolytes. Within the small intestine, most of the bile acids (>85%) are reabsorbed and returned to the liver via portal blood. Bile acid uptake from the intestinal lumen into the enterocytes is mediated largely by the apical sodium-dependent bile acid transporter, Asbt/Slc10a2, whereas the export of the bile acids from the enterocytes into portal blood is mediated largely by Ostα-Ostβ.

Dyslipidemia, including hypercholesterolemia and hypertriglyceridemia, is a common and important cluster of risk factors for coronary heart disease, hypertension, hyperinsulinemia and obesity. Obesity, in turn, is associated with an increased risk of many other diseases, including stroke and certain forms of cancer. Treatments of lipid imbalances involve a combination of altered diet and other lifestyle changes, drugs that alter lipid absorption and/or metabolism, and in some cases surgery. The three major pharmacologic approaches for treating dyslipidemias are: 1) Inhibiting cholesterol synthesis with statins; 2) Inhibiting cholesterol absorption with ezetimibe, plant stanols/sterols, polyphenols, or with nutraceuticals such as oat bran, psyllium and soy proteins; and 3) Sequestering bile acids in the intestine with non-absorbable resins. Adjuvant therapies include: fibrates to increase expression of lipoprotein lipase, niacin to raise the levels of high-density lipoprotein cholesterol (HDL-C), and drugs such as orlistat that inhibit pancreatic lipase, thereby reducing the digestion and absorption of fat in the small intestine. Although reduction of plasma cholesterol by statins is effective at diminishing the risk of coronary heart disease, such therapy is often sub-optimal, particularly in patients with reduced LDL receptors (familial hypercholesterolemia), and novel or adjuvant therapies are therefore warranted.

Given the importance of bile acids to overall lipid balance, much effort has been devoted to the development of additional strategies for altering bile acid disposition. Bile acids are physiological detergents that are synthesized in liver from cholesterol, and are delivered via the bile into the small intestine, where they play a critical role in the emulsification and intestinal absorption of dietary lipids. The bile acids themselves are largely reabsorbed in the small intestine and returned to the liver via portal blood, creating an enterohepatic cycle. The enterohepatic circulation of bile acids is critical for the absorption, transport, and distribution of lipids, and is thus a pharmacological target for the modulation of blood triglyceride, cholesterol, and other lipid levels.

A number of drugs and bile acid-derivatives have been tested as possible inhibitors of intestinal bile acid absorption, but none exhibit the required efficacy and specificity. Because bile acid transport activity can also be affected at the transcriptional level, drugs that target nuclear receptors are also currently being evaluated, although none are yet established. As noted above, one approach that has been used successfully in humans since the 1960s is the oral administration of non-absorbable bile acid-binding resins or polymers, which sequester the bile acids in the intestinal lumen and prevent their absorption. Although bile acid sequestrants are effective at limiting bile acid and lipid absorption, they have some adverse effects, including interfering in the absorption of some drugs and vitamins, altering the physicochemical state of the intestinal contents leading to constipation, and increasing plasma triglyceride levels due to alterations of hepatic lipid metabolism. To overcome some of these limitations, considerable effort has been devoted to the development of drugs that can selectively inhibit bile acid absorption, but none of the agents thus far identified are available for use in humans.

As noted above, bile acid uptake from the intestinal lumen into the enterocytes is mediated largely by the sodium-bile acid cotransporter, Asbt/Slc10a2, whereas efflux from the enterocytes into the splanchnic circulation appears to be mediated largely by the basolateral organic solute efflux transporter, Ostα-Ostβ. Ostα-Ostβ is composed of a predicted 340 amino acid, 7-transmembrane domain protein (Ostα), and a putative 128 amino acid, single-transmembrane (TM) domain ancillary polypeptide (Ostβ). Although co-expression of Ostα and Ostβ is required to elicit transport activity, the roles of the two proteins in generating the functional transporter are unknown.

It would be desirable to more fully characterize the Ostα-Ostβ transporter and its activity to identify methods of perturbing its activity, so as to alter bile acid absorption or lipid absorption (including cholesterol absorption), which can be used to treat or prevent any of a variety of dyslipidemias and associated conditions, as well as imbalances in bile acid levels.

The present invention is directed to overcoming these and other deficiencies in the art.

### SUMMARY OF THE INVENTION

One aspect of the present invention is directed to a method of treating a patient having a dyslipidemia or a disease associated with dyslipidemia. This method includes providing a therapeutic agent that inhibits Ostα-Ostβheteromeric complex formation or inhibits activity of the functional Ostα-Ostβtransporter, and administering the therapeutic agent to a patient having the dyslipidemia or a disease associated with dyslipidemia. Administration of the therapeutic agent is effective to treat the dyslipidemia or a disease associated with dyslipidemia.

A second aspect of the present invention is directed to a method of inhibiting bile acid absorption in a patient. This method includes providing a therapeutic agent that inhibits the Ostα-Ostβheteromeric complex formation or Ostα-Ostβ transport activity and administering the therapeutic agent to the patient. Administration of the therapeutic agent is effective to reduce bile acid absorption.

A third aspect of the present invention is directed to a method of inhibiting lipid absorption in a patient. This method includes providing a therapeutic agent that inhibits the Ostα-Ostβ heteromeric complex formation or Ostα-Ostβ transport activity and administering the therapeutic agent to the patient. Administration of the therapeutic agent is effective to reduce lipid absorption.

A fourth aspect of the present invention is directed to a method of treating a condition associated with altered bile acid homeostasis in a patient. This method includes providing a therapeutic agent that modulates Ostα-Ostβ heteromeric complex formation or modulates Ostα-Ostβ transporter activity, and administering the therapeutic agent to modulate bile acid transport in the patient. Administration of the therapeutic agent is effective, to modulate bile acid transport, to treat the condition associated with altered bile acid homeostasis in the patient.

A fifth aspect of the present invention relates to an isolated nucleic acid or polypeptide that binds to either (i) an Ostα or an Ostβ transmembrane domain epitope, which upon binding prevents formation of an Ostα-Ostβ heteromeric complex, or (ii) an Ostα or Ostβ transport domain, which upon binding prevents transport by the Ostα-Ostβ transporter complex. The present invention also relates to isolated nucleic acid molecules that encode a functional Ostα or an Ostβ protein, and expression vectors that encode the same, including such vectors that include a mammalian tissue-specific promotor.

A sixth aspect of the present invention relates to a pharmaceutical composition that includes an isolated nucleic acid, peptide, antibody, or small molecule of the present invention and a pharmaceutically acceptable carrier.

A seventh aspect of the present invention relates to a non-human mammal having in its germ and somatic cells an artificially induced Ostα null mutation, wherein the mammal does not express Ostα protein.

Overall, because Ostα-Ostβ plays a central role in the transport of bile acids, conjugated steroids, and structurally-related molecules across the basolateral membrane of many epithelial cells, it is a highly promising therapeutic target in a number of conditions related to imbalances in bile acid, sterol, or lipid homeostasis, or in diseases related to bile acid malabsorption, cholestasis, or cholelithiasis. As demonstrated with Ostα-deficient mice, complete disruption of the Ostα-Ostβ transporter produces a marked defect in intestinal bile acid and conjugated steroid absorption; a decrease in bile acid pool size and serum bile acid levels; altered intestinal, hepatic, and renal disposition of known substrates of the transporter; and lower serum triglyceride and cholesterol levels. Thus, as described herein and demonstrated in the accompanying examples, inhibition of this transporter should have advantages over other maneuvers that have been used to interrupt the enterohepatic circulation of bile acid.

### BRIEF DESCRIPTION OF THE DRAWINGS

FIGS. 1A-D are amino acid sequence alignments of Ostα from multiple species. FIG. 1A is an amino acid sequence alignment of the entire Ostα protein. Reference sequences for human, chimp, mouse and rat Ostα proteins were obtained from the NCBI database and were aligned using the CLUSTAL 2.0.1 program. The seven predicted transmembrane domains are boxed and the conserved cysteine-rich region is bolded. FIGS. 1B-C are amino acid sequence alignments of the individual transmembrane domains of Ostα from human, chimp, mouse, rat, dog, horse, and skate. Also shown are various consensus sequences derived from each transmembrane domain sequence alignment. These consensus sequences include a “mammalian consensus sequence-1” generated from the alignment of human, chimp, mouse, and rat Ostα transmembrane sequences, a “mammalian consensus sequence-2” generated from the alignment of human, chimp, mouse, rat, dog, and horse Ostαtransmembrane sequences, and a “consensus sequence with skate” generated from the alignment of human, chimp, mouse, rat, and skate Ostα transmembrane sequences. FIG. 1D is an amino acid sequence alignment of the cysteine-rich region of the Ostα protein located between transmembrane domains three and four. This amino acid region is conserved across human, chimp, mouse, rat, dog, horse and skate Ostα protein sequences. “*” denotes conserved amino acids among the aligned sequences; “:” denotes a conserved amino acid substitution; and “.” denotes a semi-conserved amino acid substitution.

FIGS. 2A-B are amino acid sequence alignments of Ostβ from multiple species. FIG. 2A is an amino acid sequence alignment of the entire Ostβ protein. Reference sequences for human, chimp, mouse and rat Ostβ proteins were obtained from the NCBI database and were aligned using the CLUSTAL 2.0.1 program. The predicted transmembrane domain of Ostβ is boxed and the five amino acid upstream region is bolded. FIG. 2B is an amino acid sequence alignment of the transmembrane domain and five amino acid upstream region of Ostβ from human, chimp, mouse, rat, dog, horse, and skate. Also shown are consensus sequences derived from the transmembrane domain sequence alignment. These consensus sequences include a “mammalian consensus sequence-1” generated from the alignment of human, chimp, mouse, and rat Ostβ transmembrane sequences, a “mammalian consensus sequence-2” generated from the alignment of human, chimp, mouse, rat, dog, and horse Ostβ transmembrane sequences, and a “consensus sequence with skate” generated from the alignment of human, chimp, mouse, rat, and skate Ostβ transmembrane sequences. “*” denotes conserved amino acids among the aligned sequences; “:” denotes a conserved amino acid substitution; and “.” denotes a semi-conserved amino acid substitution.

FIGS. 3A-F show glycosidase sensitivity of Ostα and Ostβ in mouse ileum, and co-immunoprecipitation of these proteins in mouse ileum and transfected HEK293 cells. Mouse ileum membrane proteins (40 μg) were incubated in the presence and absence of PNGase F, and then subjected to Western Blotting using the anti-Ostα (FIG. 3A) or anti-Ostβ (FIG. 3B) antibodies. In FIG. 3A, the open arrows indicate bands that are sensitive to PNGase F, and the solid arrows indicate the new bands formed after PNGase F treatment. Mouse ileum membrane protein samples were immunoprecipitated (IP) using anti-Ostα, anti-Ostβ, or Mrp1 antibodies, and immunoblotted (IB) using anti-Ostα (FIG. 3C) or anti-Ostβ (FIG. 3D) antibodies. HEK293 cells transfected with 1 μg plasmid DNA of Ostα-FLAG, Ostβ-c-myc, or Ostα-FLAG-IRES-Ostβ-c-myc sequences were lysed and immunoprecipitated with anti-FLAG or anti-c-myc antibody, and the immunoprecipitated fractions were collected by protein G agarose and immunoblotted using anti-Ostα (FIG. 3E) or anti-Ostβ (FIG. 3F) antibodies. In FIG. 3C-E, the arrows denote the bands that were co-immunoprecipitated. The asterisk in FIG. 3C indicates the band that was generated by the antibody used to immunoprecipitate the proteins.

FIGS. 4A-G illustrate heterodimerization and functional activity of Ostα-YN and Ostβ-YC in HEK293 cells. HEK293 cells were transfected with bJun-YN, bFos-YC, and pDsRed2-ER (FIG. 4A-C), or Ostα-YN, Ostβ-YC, and pDsRed2-ER (FIG. 4D-F), and cultured for 24 hours. The pDsRed2-ER was visualized using 543 nm excitation and 610/30 nm emission filters (FIGS. 4A and D). The BiFC complexes were visualized using 488 nm excitation and 555/15 nm emission filters (FIGS. 4B and E). The images were superimposed to evaluate the distribution of the complexes in the same cell (FIGS. 4C and F). Functional activity of the expressed proteins was examined by measuring the uptake of [H³]taurocholate (FIG. 4G). Cells were washed in KH buffer and incubated in 25 μM [H³]taurocholate for 30 min at 37° C. Cells were then washed and lysed to determine cell associated radioactivity and protein, (n=3, Mean±SEM). Asterisks denote values that are significantly different from control (pDsRed2-Mem-transfected cells), P<0.05.

FIGS. 5A-G show Ostα homodimer formation in HEK293 cells. Cells were transfected with Ostα-YN, Ostα-YC, and pDsRed2-ER (FIG. 5A-C), or Ostα-YN, Ostα-YC, Ostβ, and pDsRed2-Mem (FIGS. 5D-F), and 24 hours later the pDsRed2-ER and pDsRed2-Mem were visualized using 543 nm excitation and 610/30 nm emission filters (FIGS. 5A and D). The BiFC complexes were visualized using 488 nm excitation and 555/15 nm emission filters (FIGS. 5B and E). The images were superimposed to compare the distribution of the complexes in the same cell (FIGS. 5C and F). Functional activity was assessed by measuring the uptake of 25 μM [H³]taurocholate for 30 min at 37° C. (FIG. 5G), (n=3, Mean±SEM). Asterisks denote values that are significantly different from control (pDsRed2-Mem-transfected cells), P<0.05.

FIGS. 6A-F demonstrate that Ostβ is a type Ia transmembrane protein. Cells that had been transfected with either Ostα-FLAG and Ostβ-c-myc (FIGS. 6A, B, E and F) or Ostα-FLAG and c-myc-Ostβ (FIGS. 6C and D) were permeabilized using 0.1% saponin (FIGS. 6A, C and E) or not permeabilized (FIGS. 6B, D and F) and labeled with c-myc antibody (FIGS. 6A-D) or anti-FLAG mouse antibody (FIGS. 6E and F). The secondary antibody was anti-mouse IgG conjugated with rhodamine. The cells were visualized using an Olympus AH-2 microscope using 20× air immersion objective lenses.

FIGS. 7A-E show the membrane topology of Ostα and Ostβ as assessed by BiFC analysis. Twenty four hours before observation, HEK293 cells were transfected with both carboxyl-terminal fusion proteins Ostα-YN and Ostβ-YC (FIG. 7A), or amino- and carboxyl-terminal fusion proteins Ostα-YN and YC-Ostβ (FIG. 7B) or YN-Ostα and Ostβ-YC (FIG. 7C), or both amino-terminal fusion proteins YN-Ostα and YC-Ostβ (FIG. 7D). All cells were also transfected with pDsRed2-ER. Images of transfected HEK293 cells under YFP and DsRed2 filters were superimposed to evaluate the distribution of the putative Ost complex in the same cells (FIG. 7A-D). Functional activity was assessed by measuring the uptake of 25 μM [H³]taurocholate for 30 min at 37° C. (E), (n=3, Mean±SEM). Asterisks denote values that are significantly different from control (pDsRed2-Mem-transfected cells), P<0.05.

FIGS. 8A-H show the relative turnover rates of the Ostα and Ostβ proteins in HEK293 cells. Cells were transfected with 0.1 μg plasmid DNA containing either Ostα-FLAG, Ostβ-c-myc, or both, and after 48 hours, protein abundance was probed by Western blotting using anti-FLAG or anti-c-myc antibodies (FIGS. 8A and B). Proteins levels were also examined at 2, 8, and 24 hour without (FIGS. 8C and D) or with treatment with 100 μM cycloheximide (FIG. 8 E-H). Actin was used as a loading control.

FIGS. 9A-B show Ost/3mRNA and Ostβ protein levels in tissues from Ostα^{-/-} mice. Total RNA and proteins isolated from Ostα^{-/-} and wild-type mice were analyzed by real-time quantitative RT-PCR (FIG. 9A, n=5-6 mice for each analysis; mean±SEM), and Western blotting (FIG. 9B) using anti-Ostβ (mB91) antibody. Ostβ mRNA data are reported relative to β-actin mRNA levels in each tissue. Proteins from Ostα and Ostβ co-transfected HEK293 cells were used as a control in the Western blot (lane 1). BD=below detection limit. Actin was used as a loading control.

FIGS. 10A-B illustrate the genetic organization of the Ostβ truncations and the functional activity of Ostα-YN and Ostβ-YC or truncations of Ostβ-YC in HEK293 cells. The different truncated forms of Ostβ shown in FIG. 10A were made and inserted into the pBiFCC155 plasmid. The black bar symbolizes the relative length and the position of the predicted single transmembrane (TM) domain in the truncations. HEK293 cells were transfected with Ostα-YN, or Ostα-YN paired with either Ostβ-YC, Ostβ₁₋₁₀₇-YC, Ostβ₁₋₅₃-YC, Ostβ₂₈₋₁₂₈-YC, Ostβ₂₉₋₅₃-YC, or Ostβ_{Δ34-53} YC. After 24 hours, functional activity of the expressed proteins was examined by measuring the uptake of [³H]taurocholate (FIG. 10B). Cells were washed in KH buffer and incubated in medium containing 25 μM [³H]taurocholate for 30 min at 37° C. Cells were then washed and lysed to determine cell associated radioactivity and protein (n=5 separate experiments, mean±SEM). a=Significantly different from mock-transfected cells; b=Significantly different from Ostα-YN-transfected cells.

FIGS. 11A-X show the bimolecular fluorescence complementation analysis of heterodimerization and subcellular localization of various combinations of Ostα-YN, Ostβ-YC, or their mutants in HEK293 cells. HEK293 cells were transfected with pDsRed2-ER (FIGS. 11D-F, G-I, J-L, and V-X) or pDsRed2-MEM (FIGS. 11A-C, M-O, P-R, and S-U) as ER or plasma membrane markers, respectively. Cells were co-transfected with Ostα-YN paired with either Ostβ-YC (FIGS. 11A-C, D-F), Ostβ₁₋₁₀₇-YC (FIGS. 11G-I), Ostβ₁₋₅₃-YC (FIGS. 11J-L), Ostβ₂₈₋₁₂₈-YC (FIG. 11M-O), Ostβ₂₉₋₅₃-YC (FIG. 11P-R), or Ostβ_{Δ34-53}-YC (FIGS. 11S-U), or with Ostα_{C7/S7}-YN paired with Ostβ-YC, and cultured for 24 hours. The pDsRed2-ER was visualized using 543 nm excitation and 610/30 nm emission filters (shown in red) (FIGS. 11A, D, G, J, M, P, S, and V). The BiFC complexes were visualized using 488 nm excitation and 555/15 nm emission filters (shown in green) (FIGS. 11B, E, H, K, N, Q, T, and W). The images were superimposed to evaluate the distribution of the complexes in the same cell (FIGS. 11C, F, I, L, O, R, and X).

FIG. 12 shows inhibition of Ost α-YN and Ostβ-YC transport activity in HEK293 cells by Ostβ TM domain and Ostβ truncation constructs. HEK293 cells were transfected with 0.5 μg Ostα-YN, Ostβ-YC or Ostα-YN paired with Ostβ-YC. Cells transfected with Ostα-YN/Ostβ-YC were co-transfected with lag of various Ostβ truncation constructs. After 24 hours transfection, functional activity of the expressed proteins was examined by measuring the uptake of [H³]taurocholate. Cells were washed in KH buffer and incubated in 25 μM [H³]taurocholate for 30 min at 37° C. Cells were then washed and lysed to determine cell associated radioactivity and protein, (n=5, Mean±SEM).

FIGS. 13A-C show that mutation of the cysteine-rich region in Ostα inhibits taurocholate transport activity, but mutations in the putative ER retention signals (RRK/RNR) minimally affect function. HEK293 cells were transfected with Ostα-FLAG paired with Ostβ-c-myc, or Ostα_{RRK/AAA}-FLAG paired with Ostβ-c-myc, Ostβ_{RNR/AAA}-c-myc paired with Ostα-FLAG, Ostα_{C7/S7}-FLAG paired with Ostβ plasmids, or the individual mutants (FIGS. 13A and B). After 24 hours, co-immunoprecipitation (FIG. 13C) and functional activity of the expressed proteins (FIGS. 13A and B) was examined. a=Significantly different from cells transfected with either Ostα-FLAG or Ostβ-c-myc alone; n=4 separate experiments, mean±SEM.

FIGS. 14A-R demonstrate that disruption of the RRK sequence in Ostα or the RNR sequence in Ostβ does not influence heterodimerization or trafficking of these proteins. HEK293 cells were transfected with pDsRed2-ER as an ER marker and co-transfected with wild type Ostα-GFP (FIGS. 14A-C), Ostα_{RRK/AAA}-GFP (FIGS. 14G-I), Ostβ_{RNR/AAA}-GFP (FIGS. 14M-O), Ostα-GFP paired with Ostβ (FIG. 14D-F), Ostα_{RRK/AAA}-GFP paired with Ostβ (FIG. 14J-L), or Ostβ_{RNR/AAA}-GFP paired with Ostα (FIGS. 14P-R) and cultured for 24 hours. pDsRed2-ER was visualized under 610/30 nm emission filter. GFP was visualized under 530/10 nm filter. The images were superimposed to evaluate the distribution of GFP tagged Ostα or Ostβ protein in the cell.

FIG. 15 illustrates the decreases in taurocholate transport activity observed with mutagenesis of the five evolutionarily conserved residues of mouse Ostβ (i.e., amino acids 29-30 (ED), 34-35 (WN), and 61 (R) of SEQ ID NO: 9, FIG. 2A). HEK293 cells were transfected with 1 μg each of an Ostα construct and various Ostβ constructs. After 40 h, functional activity of the expressed proteins was assayed by measuring the uptake of 25 μM [3H]taurocholate for 30 min at 37° C. Values are from three different construct preparations, each measured in duplicate, mean±SEM. Values are adjusted for transport observed in mock transfected cells. a=Significant decreases in transport activity from Ostα-Ostβ transfected cells (P<0.05).

FIGS. 16A-F are bar graphs depicting total body weight, organ weight, and intestinal length in pre-weanling Ostα^{-/-}, Ostα^{-/+}, and Ostα^{+/+} mice. Total body weights of male and female Ostα^{+/+}, Ostα^{+/-}, and Ostα^{-/-} mice at 21d of age and as adults (2 months of age) are shown in FIGS. 16A and B. Organ weights expressed as a percentage of body weight are illustrated in FIGS. 16C and D, and the length of the intestines are shown in FIGS. 16E and F, respectively. Ostα^{-/-} mice are smaller than Ostα^{+/+} mice, and Ostα^{-/-} mice have heavier and longer small intestines. Values are means±SE, n>3. Asterisks indicate values are statistically significant from Ostα^{+/+} animals, p<0.05.

FIGS. 17A-B illustrate the decrease in bile acid pool size and serum cholesterol and triglyceride levels observed in Ostα^{-/-} mice. The bile acid pool size was assessed by measuring total bile acids in the liver, gallbladder, bile duct, and small intestine of female mice that were fasted overnight (FIG. 17A). Fecal bile acids were measured in female mice that had been fed ad libitum, and are expressed as μmol of bile acids excreted per 24 h, per 100 g body weight (FIG. 17A). Cholesterol and triglyceride levels in serum of overnight fasted female mice (FIG. 17B). Values are means±SE, n>4. Asterisks indicate values are statistically significant from Ostα^{+/+} animals, p<0.05

FIGS. 18A-D show a decrease in ileal absorption of taurocholate and estrone 3-sulfate in Ostα-deficient mice. [³H]Taurocholate, 0.01 μmol/100 μl, was injected directly into the ileal lumen of Ostα^{+/+} and Ostα^{-/-} male (FIG. 18A) and female (FIG. 18B) mice that had been fed ad libitum. Tissue distribution of [³H]taurocholate was determined after 15 min. [³H]Estrone 3-sulfate, 0.005 μmol/100 μl, was injected directly into the ileal lumen of Ostα^{+/+} and Ostα^{-/-} male (FIG. 18C) and female (FIG. 18D) mice. Tissues were collected for analysis of radioactivity after 30 min. Values are means±SE, n>3; asterisks indicate values that are statistically significant from Ostα^{+/+} animals, p<0.05.

FIGS. 19A-D show the altered distribution of estrone 3-sulfate and DHEAS in Ostα-deficient mice. Tissue distribution of [³H]estrone 3-sulfate in male (FIG. 19A) and female (FIG. 19B) mice that were injected intraperitoneally with 0.1 μmol/200 μl of this compound. Tissues were collected for analysis of radioactivity after 2h. [³H]Estrone 3-sulfate, 0.4 μmol/400 μl, was injected intraperitoneally into male mice and tissues were collected for analysis of radioactivity after 2h (FIG. 19C). [³H]DHEAS, 0.4 μmol/400 μl, was injected intraperitoneally into male mice and tissues were collected for analysis of radioactivity after 2h (FIG. 19D). All of the animals for these studies were fed ad libitum until the time of experimentation. Values are means±SE, n>3; asterisks indicate values that are statistically significant from Ostα^{+/+} animals, p<0.05.

FIGS. 20A-C show gene expression in small intestine, liver, and kidney in Ostα^{-/-} animals. Total RNA of these tissues was isolated from Ostα^{+/+} and Ostα^{-/-} female mice that had been fed ad libitum, and mRNA levels were analyzed via real time RT-PCR. mRNA expression levels in the small intestine (FIG. 20A), liver (FIG. 20B), and kidney (FIG. 20C) are functionally categorized into uptake, regulation, metabolism (Met), and excretion groupings and reported relative to the expression in Ostα^{+/+} animals. All samples were analyzed in triplicate and values are means±SE, n=4 experiments. Asterisks indicate values that are statistically significant from Ostα^{+/+} animals, p<0.05; BD, below detection limit.

FIGS. 21A-B illustrate the alterations of intestinal gene expression patterns in Ostα^{-/-} animals. The small intestine of female Ostα^{+/+} and Ostα^{-/-} mice was divided into 3 sections of equal length. Total RNA was isolated, and expression levels of the indicated genes were subsequently determined by real time RT-PCR. FIG. 21A shows mRNA expression levels of the apical sodium-dependent bile acid transporter (ASBT) in the proximal, middle, and distal segments of the small intestine. Changes in Asbt expression in the Ostα^{+/+} and Ostα^{-/-} mice are reported relative to β-actin. FIG. 21B shows individual gene expression levels along the intestinal tract relative to gene expression levels in wild type animals. Samples were analyzed in triplicate and values are means±SE, n=4. Asterisks indicate values that are statistically significant from Ostα^{+/+} animals, p<0.05; BD, below detection limit.

### DETAILED DESCRIPTION OF THE INVENTION

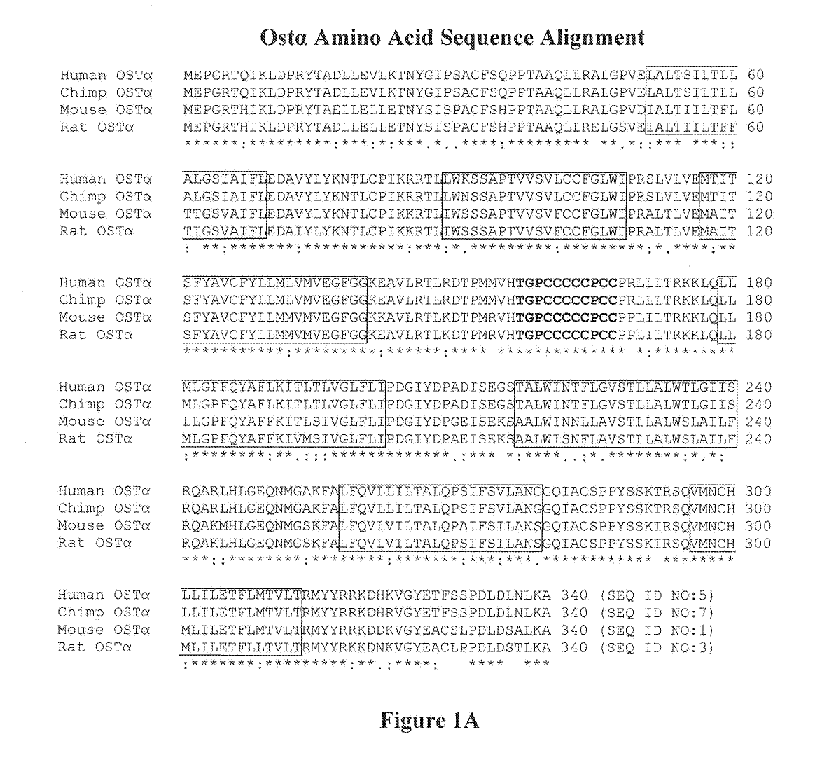

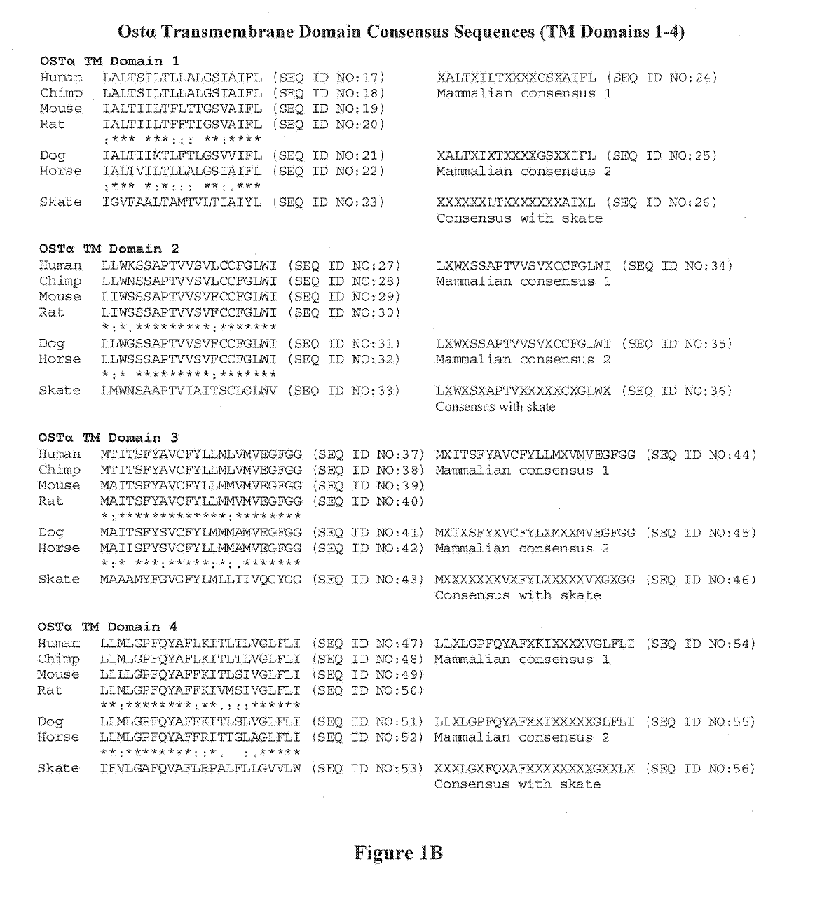

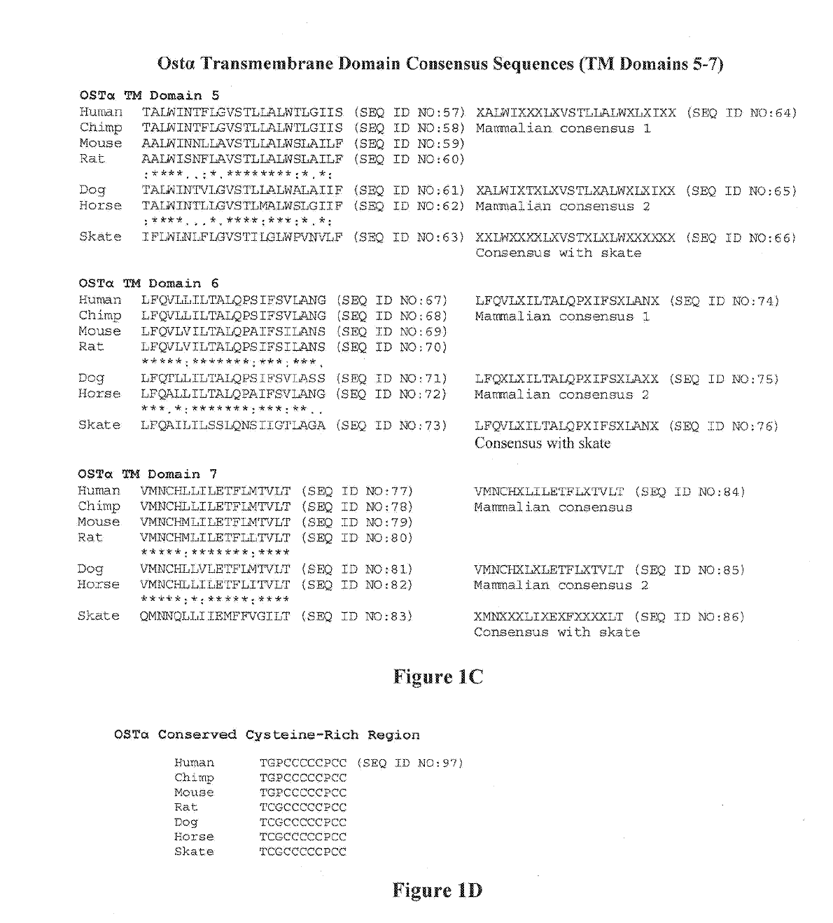

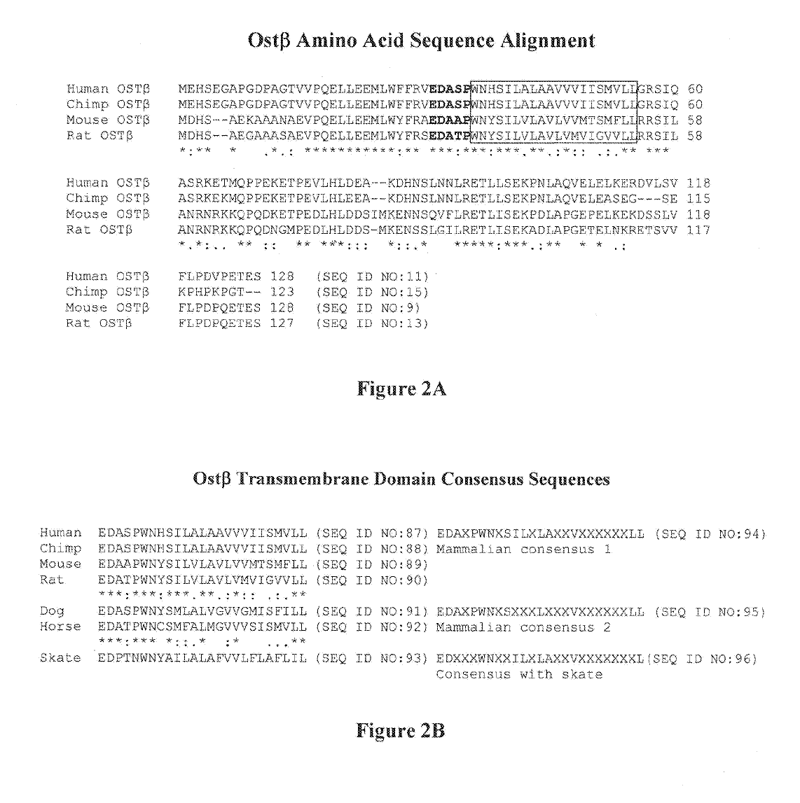

The present invention relates to various uses of therapeutic agents that target the organic solute and steroid transporter, Ostα-Ostβ, either inhibiting its heteromeric complex formation or functional transporter activity in certain tissues or enhancing its expression (and function) in certain tissues. Ostα-Ostβ is an unusual heteromeric carrier that plays a central role in the transport of bile acids, conjugated steroids, and structurally related molecules across the basolateral membrane of many cells, including hepatocytes, cholangiocytes, enterocytes, and renal tubular epithelial cells. As illustrated in FIGS. 1 and 2, as well as below, the amino acid and nucleic sequences for a number of Ostα and Ostβ homologs are well known in the art.

An exemplary mouse Ostα (Genbank Accession NM_{—}145932) amino acid sequence is shown in FIG. 1A (SEQ ID NO: 1). Its encoding nucleotide sequence (SEQ ID NO: 2) is shown below:

| | |
|---|---|
| atggagccag gcaggactca tatcaaactt gaccccaggt acacagcaga gcttctggaa | 60 |
| cttctggaga ccaattacag catctcccct gcctgcttct ctcaccctcc caccgcagcc | 120 |
| cagctcctga gagcactggg cccagtggac atagccctca ccatcatctt gacctttctt | 180 |
| accactggct cagttgccat ttttctggag gatgctgttt acctatacaa gaacaccctt | 240 |
| tgccccatca agaagaggac tctgatctgg agtagctctg cacccacggt ggtatctgtg | 300 |
| ttctgctgct ttggtctctg gattcctcgc gccctcacac ttgtagaaat ggccataacc | 360 |
| tcgttttatg ccgtatgctt ttacctgctg atgatggtca tggtggaagg ctttggtggg | 420 |
| aagaaagcag tactgaggac actgaaggac accccgatga gggtgcacac gggtccctgt | 480 |
| tgctgctgct gtccctgctg cccacctctc atacttacca gaaagaagct gcagctgctc | 540 |
| ctgctgggcc ctttccagta cgccttcttc aagataacgc tgagcatagt gggcctgttc | 600 |
| ctcatccctg acggcatcta tgacccagga gaaatttctg agaagagcgc agctctctgg | 660 |
| atcaacaatc tccttgctgt gtctaccctt ctggccctct ggtccctggc catccttttc | 720 |
| cgtcaagcca agatgcatct gggtgaacag aacatgggat ccaagtttgc tctgttccag | 780 |
| gtgcttgtca tcctgaccgc cctgcagcct gccattttct ccatcttggc taacagtggg | 840 |
| cagatcgctt gctcacctcc ctactcttct aaaatcaggt ctcaagtgat gaactgccac | 900 |
| atgctcatac tggagacctt cctgatgaca gtgctgacac gaatgtacta tcgaaggaaa | 960 |
| gacgacaagg ttgggtatga ggcttgttcg ctgccagacc tggactcagc actcaaagcc | 1020 |
| tga | 1023 |

An exemplary rat Ostα (Genbank Accession NM_{—}001107087) amino acid sequence is shown in FIG. 1A (SEQ ID NO: 3). Its encoding nucleotide sequence (SEQ ID NO: 4) is shown below:

| | |
|---|---|
| atggagccag gcaggactca tataaaactt gaccccaggt acacagcaga tcttctggag | 60 |
| ctgctggaaa ccaattacag catctcccct gcctgcttct ctcaccctcc cactgcagcc | 120 |
| cagctcctga gagaattggg ctcagtggaa attgccctca ccatcatctt gacctttttt | 180 |
| accattggat cagttgccat ttttctggag gatgctattt acctatacaa gaacaccctt | 240 |
| tgccccatca agaagaggac tctgatctgg agtagctctg cgcccacggt ggtgtctgtg | 300 |
| ttctgctgct ttggtctctg gatcccacgt gccctcacac ttgtggaaat ggccataacc | 360 |
| tcgttttatg ccgtatgctt ttacctgctg atgatggtca tggtggaagg ctttggtggg | 420 |
| aaggaagcag tactaaggac actgaaggac accccaatga gggtgcacac aggtccctgc | 480 |
| tgctgctgct gtccctgctg cccacccctc atacttacca ggaagaagct acagttgctc | 540 |
| atgttgggcc ctttccagta tgccttcttc aagatagtga tgagcatagt gggcctgttt | 600 |
| ctcattcctg atggcatcta tgacccagca gaaatttctg agaagagcgc agctctctgg | 660 |
| atcagcaatt tccttgctgt gtccaccctt ttggctctct ggtccctggc catccttttc | 720 |
| cgtcaagcca agttgcacct gggtgaacag aatatgggat ccaagtttgc tctgttccag | 780 |
| gtgcttgtca tcctgaccgc cctgcagccc tccattttct ccatcttggc taacagcggg | 840 |
| cagatcgctt gctcacctcc ctattcttct aaaatcaggt cccaagtgat gaactgtcac | 900 |
| atgctcatcc tggagacctt cctgttgaca gtgctgacac gcatgtacta tcgaaagaaa | 960 |
| gataacaagg ttgggtatga ggcttgttta ccgccagacc tggactcaac gctcaaagcc | 1020 |
| tga | 1023 |

An exemplary human Ostα (Genbank Accession NM_{—}152672) amino acid sequence is shown in FIG. 1A (SEQ ID NO: 5). Its encoding nucleotide sequence (SEQ ID NO: 6) is shown below:

| | |
|---|---|
| atggagccgg gcaggaccca gataaagctt gaccccaggt acacagcaga tcttctggag | 60 |
| gtgctgaaga ccaattacgg catcccctcc gcctgcttct ctcagcctcc cacagcagcc | 120 |
| caactcctga gagccctggg ccctgtggaa cttgccctca ctagcatcct gaccttgctg | 180 |
| gcgctgggct ccattgccat cttcctggag gatgccgtct acctgtacaa gaacaccctt | 240 |
| tgccccatca agaggcggac tctgctctgg aagagctcgg cacccacggt ggtgtctgtg | 300 |
| ctgtgctgct ttggtctctg gatccctcgt tccctggtgc tggtggaaat gaccatcacc | 360 |
| tcgttttatg ccgtgtgctt ttacctgctg atgctggtca tggtggaagg ctttgggggg | 420 |
| aaggaggcag tgctgaggac gctgagggac accccgatga tggtccacac aggcccctgc | 480 |
| tgctgctgct gcccctgctg tccacgtctg ctgctcacca ggaagaagct tcagctgctg | 540 |
| atgttgggcc ctttccaata cgccttcttg aagataacgc tgaccctggt gggcctgttt | 600 |
| ctcatccccg acggcatcta tgacccagca gacatttctg aggggagcac agctctatgg | 660 |
| atcaacactt tcctcggcgt gtccacactg ctggctctct ggaccctggg catcatttcc | 720 |
| cgtcaagcca ggctacacct gggtgagcag aacatgggag ccaaatttgc tctgttccag | 780 |
| gttctcctca tcctgactgc cctacagccc tccatcttct cagtcttggc caacggtggg | 840 |
| cagattgctt gttcgcctcc ctattcctct aaaaccaggt ctcaagtgat gaattgccac | 900 |
| ctcctcatac tggagacttt tctaatgact gtgctgacac gaatgtacta ccgaaggaaa | 960 |
| gaccacaagg ttgggtatga aactttctct tctccagacc tggacttgaa cctcaaagcc | 1020 |
| taa | 1023 |

An exemplary chimp Ostα (Genbank Accession XM_{—}516971) amino acid sequence is shown in FIG. 1A (SEQ ID NO: 7). Its encoding nucleotide sequence (SEQ ID NO: 8) is shown below:

| | |
|---|---|
| atggagccgg gcaggaccca gataaagctt gaccccaggt acacagcaga tcttctggag | 60 |
| gtgctgaaga ccaattacgg catcccctcc gcctgcttct ctcagcctcc cacagcagcc | 120 |
| caactcctga gagccctggg ccctgtggaa cttgccctca ccagcatcct gaccttgctg | 180 |
| gcgctgggct ccattgccat cttcctggag gatgccgtct acctgtacaa gaacaccctt | 240 |
| tgccccatca agaggcggac tctgctctgg aacagctcgg cacccacggt ggtgtctgtg | 300 |
| ctgtgctgct ttggtctctg gatccctcgt tccctggtgc tggtggaaat gaccatcacc | 360 |
| tcgttttatg ccgtgtgctt ttacctgctg atgctggtca tggtggaagg ctttgggggg | 420 |
| aaggaggcag tgctgaggac gctgagggac accccgatga tggtccacac aggcccctgc | 480 |
| tgctgctgct gcccctgctg tccgcggctg ctgctcacca ggaagaagct tcagctgctg | 540 |
| atgttgggcc ctttccaata cgccttcttg aagataacgc tgaccctggt gggcctgttt | 600 |
| ctcatccccg acggcatcta tgacccagca gacatttctg aggggagcac agctctatgg | 660 |
| atcaacactt tcctcggcgt gtccacactg ctggctctct ggaccctggg catcatttcc | 720 |
| cgtcaagcca ggctacacct gggtgagcag aacatgggag ccaaatttgc tctgttccag | 780 |
| gttcttctca tcctgactgc cctacagccc tccatcttct cagtcttggc caacggtggg | 840 |
| cagattgctt gttcgcctcc ctattcctct aaaaccaggt ctcaagtgat gaattgccac | 900 |
| ctcctcatac tggagacttt tctaatgact gtgctgacac gaatgtacta ccgaaggaaa | 960 |
| gaccacaggg ttgggtatga aactttctct tctccagacc tggacttgaa cctcaaagcc | 1020 |
| taa | 1023 |

An exemplary mouse Ostβ (Genbank Accession NM_{—}178933) amino acid sequence is shown in FIG. 2A (SEQ ID NO:9). Its encoding nucleotide sequence (SEQ ID NO: 10) is shown below:

| | |
|---|---|
| atggaccaca gtgcagagaa agctgcagcc aatgccgagg tgccccagga actgctggaa | 60 |
| gaaatgcttt ggtattttcg tgcagaagat gcggctcctt ggaattattc catcctggtc | 120 |
| ctggcagtcc tggtggtcat gacaagcatg ttcctcctga gaaggagcat cctggcaaac | 180 |
| agaaatcgaa agaagcagcc acaagacaaa gaaacaccag aagacctgca tcttgatgac | 240 |
| tccataatga aagagaacaa cagccaggtc ttcctaagag agacattgat ctcagagaaa | 300 |
| ccagacttgg ccccggggga acctgagttg aaagagaaag actcatcact tgtctttctg | 360 |
| ccagacccac aggaaacaga gagctag | 387 |

An exemplary human Ostβ (Genbank Accession NM_{—}178859) amino acid sequence is shown in FIG. 2A (SEQ ID NO: 11). Its encoding nucleotide sequence (SEQ ID NO: 12) is shown below:

| | |
|---|---|
| atggagcaca gtgagggggc tcccggagac ccagccggta ctgtggtacc ccaggagctg | 60 |
| ctggaagaga tgctttggtt ttttcgtgtg gaagatgcat ctccctggaa tcattccatc | 120 |
| cttgccctgg cagctgtggt ggtcattata agcatggtcc tcctgggaag aagcatccag | 180 |
| gcaagcagaa aagaaacgat gcagccacca gaaaaagaaa ctccagaagt cctgcatttg | 240 |
| gatgaggcca aggatcacaa cagcctaaac aacctaagag aaactttgct ctcagaaaag | 300 |
| ccaaacttgg cccaggtgga acttgagtta aaagagagag atgtgctgtc agttttcctt | 360 |
| ccggatgtac cagaaactga gagctag | 387 |

An exemplary rat Ostβ (Genbank Accession XM_{—}238546) amino acid sequence is shown in FIG. 2A (SEQ ID NO: 13). It encoding nucleotide sequence (SEQ ID NO: 14) is shown below:

| | |
|---|---|
| atggaccaca gtgcagaagg agctgcagcc agtgccgagg tgccccagga gctgctggag | 60 |
| gaaatgcttt ggtatttccg ttcagaggat gcaactcctt ggaactattc catcctggtt | 120 |
| ctggcagtcc tggtgatggt gataggcgtg gtcctcctga gaaggagcat cctggcaaac | 180 |
| agaaatcgaa agaagcagcc acaagacaac ggaatgccag aagacctgca tctagatgat | 240 |
| tccatgaaag aaaacagcag cctgggcatc ctaagagaga cgctgatctc agagaaggca | 300 |
| gacttggccc caggggaaac cgagttgaac aagagagaga catcagtcgt ctttctacca | 360 |
| gacccccagg aaactgagag ctag | 384 |

An exemplary chimp Ostβ (Genbank Accession XM_{—}001174338) amino acid sequence is shown in FIG. 2A (SEQ ID NO: 15). It encoding nucleotide sequence (SEQ ID NO: 16) is shown below:

| | |
|---|---|
| atggagcaca gtgagggggc tcccggagac ccagccggta ctgtggtgcc ccaggagctg | 60 |
| ctggaagaga tgctttggtt ttttcgggtg gaagatgcat ctccctggaa tcattccatc | 120 |
| cttgccctgg cagctgtggt ggtcattata agcatggtcc tcctgggaag aagcatccag | 180 |
| gcaagcagaa aagaaaagat gcagccacca gaaaaagaaa ctccagaagt cctgcatttg | 240 |
| gaagaggcca aggatcacaa cagcctaaac aacctaagag aaactttgct ctcagaaaag | 300 |
| ccaaacttgg cccaggtgga acttgaagct agtgagggtt cagagaagcc ccatcctaag | 360 |
| ccaggcacat ga | 372 |

One aspect of the present invention is directed to a method of treating a patient having a dyslipidemia or a disease associated with dyslipidemia. This method includes providing a therapeutic agent that inhibits Ostα-Ostβ heteromeric complex formation or inhibits activity of the functional Ostα-Ostβ transporter, and administering the therapeutic agent to a patient having the dyslipidemia or a disease associated with dyslipidemia. Administration of the therapeutic agent is effective to treat the dyslipidemia or a disease associated with dyslipidemia.

In accordance with this aspect of the present invention, a patient suffering from dyslipidemia may have hypercholesterolemia, hypertriglyceridemia, and/or hyperlipidemia. In addition, patients suffering from any disease associated with or caused by dyslipidemia are also amenable to treatment using the methods of the present invention. Diseases typically associated with dyslipidemia include, but are not limited to, obesity, stroke, atherosclerosis, coronary heart disease, hypertension, and hyperinsulinemia.

As used herein, “patient” refers to any animal that exhibits a condition, such as dyslipidemia or any other condition described infra, which is associated with a disruption in bile acid homeostasis and is therefore amenable to treatment in accordance with the methods of the present invention. Preferably, the patient is a mammal. Exemplary mammalian patients include, without limitation, humans, non-human primates, dogs, cats, rodents (e.g., mouse, rat, guinea pig), horses, cattle and cows, sheep, and pigs.

In accordance with this aspect of the present invention, a therapeutic agent that is effective for treating a patient having a dyslipidemia or a disease associated with dyslipidemia is an agent that reduces uptake or absorption of dietary lipids and cholesterol. Therefore, an effective therapeutic agent of the present invention reduces a patients lipid and cholesterol blood levels.

A second aspect of the present invention is directed to a method of inhibiting lipid absorption in a patient. This method includes providing a therapeutic agent that inhibits the Ostα-Ostβ heteromeric complex formation or Ostα-Ostβ transport activity and administering the therapeutic agent to the patient.

Administration of the therapeutic agent is effective to reduce lipid absorption. In accordance with this aspect of the present invention inhibition of lipid absorption includes inhibition of any fat-soluble, naturally occurring molecule, such as fats, oils, waxes, cholesterol, sterols, fat-soluble vitamins, including vitamins A, D, E, and K, monoglycerides, diglycerides, triglycerides, phospholipids, and others.

A third aspect of the present invention is directed to a method of inhibiting bile acid absorption in a patient. This method includes providing a therapeutic agent that inhibits the Ostα-Ostβ heteromeric complex formation or Ostα-Ostβ transport activity and administering the therapeutic agent to the patient. Administration of the therapeutic agent is effective to reduce bile acid absorption.

In accordance with this aspect of the invention, inhibition of bile acid absorption in a patient enhances either urinary or fecal excretion of bile acids. Accordingly, a preferred therapeutic agent inhibits bile acid absorption from the small intestine and/or kidneys. Greater than 85% of bile acids are reabsorbed from the intestine and returned to the liver via portal circulation in the small intestine. While bile acid uptake from the intestinal lumen is mediated by the apical sodium dependent bile acid transporter, bile acid export from the enterocytes into the splanchinic circulation is mediated largely by the Ostα-Ostβ transporter. Likewise, while bile acid uptake from the renal tubular lumen is mediated by the apical sodium dependent bile acid transporter, bile acid export from the renal tubular cells into the peritubular circulation is mediated largely by the Ostα-Ostβ transporter. Therapeutic agents which inhibit Ostα-Ostβ transport activity, either directly or via inhibition of heterocomplex formation, will effectively reduce bile acid absorption via the small intestine and/or kidneys.

The therapeutic agent useful for carrying out the methods of the present invention can be an agent that inhibits Ostα-Ostβ heteromeric complex formation. Therapeutic agents which inhibit Ostα-Ostβ heteromeric complex formation are agents which bind to a region within or near the Ostβ transmembrane domain or a region within or near one of the seven Ostα transmembrane domains, and thereby prevent complex formation. Such agents prevent the Ostα-Ostβ complex formation and, due to protein instability, promote otherwise natural degradation of one or both proteins. This has the effect of limiting or eliminating the presentation of a functional Ostα-Ostβ transporter. Alternatively, the therapeutic agent may inhibit Ostα or Ostβ translation and expression, also preventing complex formation. Suitable therapeutic agents include protein or peptides (i.e. antibodies and inhibitory peptides), nucleic acids molecules (i.e. RNAi, antisense molecules, aptamers, and nucleic acids encoding antibodies and inhibitory peptides), and small molecules.

In an alternative embodiment of the present invention, the therapeutic agent can inhibit Ostα-Ostβ transporter activity. That is, the functional transporter can be present in the cellular membrane, but its activity (i.e., ability to absorb bile acids, sterol, etc.) is disrupted. Therapeutic agents which inhibit Ostα-Ostβ transport activity are agents which bind to regions of the Ostα protein involved in ligand recognition or binding, transporter formation, trafficking to the cell surface, or other aspects of transport activity. The regions of Ostα protein important in modulating transport activity include the extracellular N-terminal region of Ostα as well as the internal cysteine-rich domain located between transmembrane domains three and four of Ostα. Alternatively, the therapeutic agent may target the N-terminal region (i.e., amino acids 1-27) of the Ostβ protein. Suitable therapeutic agents include protein or peptides (i.e. antibodies and inhibitory peptides), nucleic acids molecules (i.e. RNAi, antisense molecules, aptamers, and nucleic acids encoding antibodies and inhibitory peptides), and small molecules.

As described supra, in one embodiment of the present invention, the therapeutic agent that inhibits Ostα-Ostβ heteromeric complex formation is a protein or peptide. Exemplary protein or peptide therapeutic agents include inhibitory peptides, aptamers, and antibodies.

Inhibitory peptides of the present invention include peptides that bind to an Ostα transmembrane domain region. Suitable peptides that bind to an Ostαtransmembrane domain region include those peptides that mimic at least a portion of the Ostβ transmembrane region. Accordingly, the peptides of the present invention may mimic a portion of the amino acid region upstream of the Ostβ transmembrane domain, or a portion of the Ostβ transmembrane domain and the amino acid region upstream. Because the Ostβ TM domain and the amino acid region upstream is critical for Ostα-Ostβ heterocomplex formation, peptides mimicking this region will interfere with, and prevent, endogenous heterocomplex formation, and thus inhibit functional activity. Accordingly, suitable inhibitory peptides include, but are not limited to, those peptides having an amino acid sequence of SEQ ID NO:87 (corresponding to human OstβTM domain and upstream amino acid region), SEQ ID NO:88 (corresponding to chimp Ostβ TM domain and upstream amino acid region), SEQ ID NO:89 (corresponding to mouse Ostβ TM domain and upstream amino acid region), SEQ ID NO:90 (corresponding to rat Ostβ TM domain and upstream amino acid region), SEQ ID NO:91 (corresponding to dog Ostβ TM domain and upstream amino acid region), SEQ ID NO:92 (corresponding to horse Ostβ TM domain and upstream amino acid region), or SEQ ID NO:93 (corresponding to skate Ostβ TM domain and upstream amino acid region). Alternatively, the inhibitory peptide may be derived from a consensus sequence of the Ostβ TM domain and amino acid region upstream having an amino acid sequence of SEQ ID NO: 94, SEQ ID NO:95, or SEQ ID NO:96 (See FIG. 2B). In these consensus sequences, the X residues can be any amino acid, but preferably a neutral or hydrophobic amino acid (the latter being most preferred). Additionally, peptide fragments that comprise at least 50 percent, more preferably at least 75 or 80 or 85 percent, most preferably at least 90 or 95 percent of contiguous amino acid residues from any one of SEQ ID NOs:87-96 are also suitable.

Inhibitory peptides of the present invention also include peptides that bind to an Ostβ transmembrane domain region. Suitable peptides that bind to an Ostβ transmembrane domain region include those peptides that mimic at least a portion of the Ostα transmembrane region. In a preferred embodiment, the inhibitory peptide of the present invention mimics a region of an Ostα transmembrane domain that interacts with the Ostβ transmembrane domain region, i.e. an interaction domain. Accordingly, suitable inhibitory peptides include those peptides having an amino acid sequence corresponding to Ostα TM domain 1 (SEQ ID NOs:17-23) or a consensus sequence thereof (SEQ ID NOs:24-26); Ostα TM domain 2 (SEQ ID NOs:27-33) or a consensus sequence thereof (SEQ ID NO:34-36); Ostα TM domain 3 (SEQ ID NOs:37-43) or a consensus sequence thereof (SEQ ID NO:44-46); Ostα™ domain 4 (SEQ ID NOs:47-53) or a consensus sequence thereof (SEQ ID NO:54-56); Ostα TM domain 5 (SEQ ID NOs:57-63) or a consensus sequence thereof (SEQ ID NO:64-66); Ostα™ domain 6 (SEQ ID NOs:67-73) or a consensus sequence thereof (SEQ ID NO:74-76); Ostα TM domain 7 (SEQ ID NOs:77-83) or a consensus sequence thereof (SEQ ID NO:84-86). In the consensus sequences, the X residues can be any amino acid, but preferably a neutral or hydrophobic amino acid (the latter being most preferred). Additionally, peptide fragments that comprise at least 50 percent, more preferably at least 75 or 80 or 85 percent, most preferably at least 90 or 95 percent of contiguous amino acid residues from any one of SEQ ID NOs:17-86 are also suitable.

The peptides of the present invention can also be in the form of a fusion protein that includes a sequence that interferes with Ostα-Ostβ heterocomplex formation or transport activity and one or more additional sequences that (i) promote cellular uptake, (ii) target the peptide to a particular cell type, or (iii) target the peptide to a specific sub-cellular localization after cell uptake. Exemplary sequences of these types are described infra. The generation of fusion proteins, and their encoding nucleic acid molecules, via recombinant molecular techniques are well known in the art.

The peptides of the present invention may be prepared using standard methods of synthesis known in the art, including solid phase peptide synthesis (Fmoc or Boc strategies) or solution phase peptide synthesis. Alternatively, peptides of the present invention may be prepared using recombinant expression systems.

Generally, the use of recombinant expression systems involves inserting the nucleic acid molecule encoding the amino acid sequence of the inhibitory peptide into an expression system to which the molecule is heterologous (i.e., not normally present). One or more desired nucleic acid molecules encoding a peptide of the invention may be inserted into the vector. When multiple nucleic acid molecules are inserted, the multiple nucleic acid molecules may encode the same or different peptides. The heterologous nucleic acid molecule is inserted into the expression system or vector in proper sense (5′→3′) orientation relative to the promoter and any other 5′ regulatory molecules, and correct reading frame.

The nucleic acid molecules can be derived from the known Ostα and Ostβ nucleic acid sequences using the above-referenced Genbank Accessions or consensus sequences (or by using hereafter identified Ostα and Ostβ nucleic acid sequences). In certain embodiments, it may be desirable to prepare codon-enhanced nucleic acids that will favor expression of the desired peptide in the transgenic expression system of choice.

The preparation of the nucleic acid constructs can be carried out using standard cloning procedures well known in the art as described by Sambrook et al., Molecular Cloning: A Laboratory Manual, Cold Springs Laboratory, Cold Springs Harbor, N.Y. (1989). U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in a suitable host cell.

A variety of genetic signals and processing events that control many levels of gene expression (e.g., DNA transcription and messenger RNA (“mRNA”) translation) can be incorporated into the nucleic acid construct to maximize peptide production. DNA transcription, for example, is dependent upon the presence of a promoter, which is a DNA sequence that directs the binding of RNA polymerase, and thereby promotes mRNA synthesis. Promoters vary in their “strength” (i.e., their ability to promote transcription). For the purposes of expressing a cloned gene, it is desirable to use strong promoters to obtain a high level of transcription and, hence, expression of the gene. Depending upon the host system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the P_{R} and P_{I}, promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Common promoters suitable for directing expression in mammalian cells include, without limitation, SV40, MMTV, metallothionein-1, adenovirus Ela, CMV, immediate early, immunoglobulin heavy chain promoter and enhancer, and RSV-LTR.

Specific initiation signals are also required for efficient gene transcription and translation in prokaryotic cells. These transcription and translation initiation signals may vary in “strength” as measured by the quantity of gene specific messenger RNA and protein synthesized, respectively. The nucleic acid expression vector, which contains a promoter, may also contain any combination of various “strong” transcription and/or translation initiation signals. For instance, efficient translation in E. coli requires a Shine-Dalgarno (“SD”) sequence about 7-9 bases 5′ to the initiation codon (ATG) to provide a ribosome binding site. Thus, any SD-ATG combination that can be utilized by host ribosomes may be employed. Such combinations include but are not limited to the SD-ATG combination from the cro gene or the N gene of coliphage lambda, or from the E. coli tryptophan E, D, C, B or A genes. Additionally, any SD-ATG combination produced by recombinant DNA or other techniques involving incorporation of synthetic nucleotides may be used. Depending on the vector system and host utilized, any number of suitable transcription and/or translation elements, including constitutive, inducible, and repressible promoters, as well as minimal 5′ promoter elements, enhancers or leader sequences may be used. For a review on maximizing gene expression see Roberts and Lauer, Methods in Enzymology, 68:473 (1979), which is hereby incorporated by reference in its entirety.

When multiple nucleic acid molecules are inserted into an expression vector, the multiple nucleic acid molecules may all be placed under a single 5′ regulatory region and a single 3′ regulatory region, where the regulatory regions are of sufficient strength to transcribe and/or express the nucleic acid molecules as desired.

A nucleic acid molecule encoding a suitable Ostα or Ostβ inhibitory peptide, a promoter molecule of choice, including, without limitation, enhancers, and leader sequences; a suitable 3′ regulatory region to allow transcription in the host, and any additional desired components, such as reporter or marker genes, are cloned into the vector of choice using standard cloning procedures in the art, such as described in Sambrook et al., Molecular Cloning: A Laboratory Manual, Cold Spring Laboratory, Cold Spring Harbor, N.Y. (1989); Ausubel et al., “Short Protocols in Molecular Biology,” New York:Wiley (1999), and U.S. Pat. No. 4,237,224 to Cohen and Boyer, which are hereby incorporated by reference in their entirety.

Once the nucleic acid molecule encoding the peptide has been cloned into an expression vector, it is ready to be incorporated into a host. Recombinant molecules can be introduced into cells, without limitation, via transfection (if the host is a eukaryote), transduction, conjugation, mobilization, or electroporation, lipofection, protoplast fusion, mobilization, or particle bombardment, using standard cloning procedures known in the art, as described by Sambrook et al., Molecular Cloning: A Laboratory Manual, Second Edition, Cold Springs Laboratory, Cold Springs Harbor, N.Y. (1989), which is hereby incorporated by reference in its entirety.

A variety of suitable host-vector systems may be utilized to express the recombinant protein or polypeptide. Primarily, the vector system must be compatible with the host used. Host-vector systems include, without limitation, the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria. The expression elements of these vectors vary in their strength and specificities. Depending upon the host-vector system utilized, any one of a number of suitable transcription and translation elements can be used to carry out this and other aspects of the present invention.

Purified peptides may be obtained by several methods readily known in the art, including ion exchange chromatography, hydrophobic interaction chromatography, affinity chromatography, gel filtration, and reverse phase chromatography. The peptide is preferably produced in purified form (preferably at least about 80% or 85% pure, more preferably at least about 90% or 95% pure) by conventional techniques. Depending on whether the recombinant host cell is made to secrete the peptide into growth medium (see U.S. Pat. No. 6,596,509 to Bauer et al., which is hereby incorporated by reference in its entirety), the peptide can be isolated and purified by centrifugation (to separate cellular components from supernatant containing the secreted peptide) followed by sequential ammonium sulfate precipitation of the supernatant. The fraction containing the peptide is subjected to gel filtration in an appropriately sized dextran or polyacrylamide column to separate the peptides from other proteins. If necessary, the peptide fraction may be further purified by HPLC.

Alternatively, if the peptide of interest is not secreted, it can be isolated from the recombinant cells using standard isolation and purification schemes. This includes disrupting the cells (e.g., by sonication, freezing, French press, etc.) and then recovering the peptide from the cellular debris. Purification can be achieved using the centrifugation, precipitation, and purification procedures described above and in Philip L. R. Bonner, Protein Purification (Routledge 2007), which is hereby incorporated by reference in its entirety.

Whether the peptide of interest is secreted or not, it may also contain a purification tag (such as poly-histidine (His₆), a glutathione-S-transferase (GST-), or maltose-binding protein (MBP-)), which assists in the purification but can later be removed, i.e., cleaved from the peptide following recovery. Protease-specific cleavage sites can be introduced between the purification tag and the desired peptide. The desired peptide product can be purified further to remove the cleaved purification tags.

Other suitable therapeutic agents of the present invention include antibodies which are capable of binding to a particular epitope of Ostα or Ostβ, preferably one of the seven transmembrane domains of Ostα or the transmembrane domain region of Ostβ (see FIGS. 1B-C and FIG. 2B). The disclosed antibodies may be monoclonal, polyclonal, or active fragments thereof.

Monoclonal antibody production may be effected by techniques which are well-known in the art. Monoclonal Antibodies-Production, Engineering and Clinical Applications, Ritter et al., Eds. Cambridge University Press, Cambridge, UK (1995), which is hereby incorporated by reference in its entirety. Basically, the process involves first obtaining immune cells (lymphocytes) from the spleen of a mammal (e.g., mouse) which has been previously immunized with the antigen of interest (i.e., a peptide fragment of Ostα or Ostβ containing the epitope of interest) either in vivo or in vitro. Exemplary Ostα and Ostβ peptides for generating antibodies of the present invention are peptide sequences corresponding to the Ostαtransmembrane domains shown in FIGS. 1B-C (i.e. SEQ ID NO: 17-86) and peptide sequences corresponding to the Ostβ transmembrane domain shown in FIG. 2B (i.e. SEQ ID NO: 87-96). Alternatively, an Ostα peptide comprising the conserved cysteine-rich region between transmembrane domain three and four may be used to generate an antibody that inhibits Ostα-Ostβ transport activity. Ideally, this Ostαpeptide would comprise at least a portion of the amino acid sequence of SEQ ID NO:97 (FIG. 1D). Likewise, an Ostβ peptide comprising at least a portion of the N-terminal amino acid sequence (i.e. amino acid residues 1-27) of SEQ ID NO: 9, 11, 13, or 15, would also be suitable for generating an antibody that inhibits Ostα-Ostβ transport activity.

The antibody-secreting lymphocytes are then fused with (mouse) myeloma cells or transformed cells, which are capable of replicating indefinitely in cell culture, thereby producing an immortal, immunoglobulin-secreting cell line. Fusion with mammalian myeloma cells or other fusion partners capable of replicating indefinitely in cell culture is effected by standard and well-known techniques, for example, by using polyethylene glycol (PEG) or other fusing agents (Milstein and Kohler, “Derivation of Specific Antibody-Producing Tissue Culture and Tumor Lines by Cell Fusion,” Eur. I Immunol., 6:511 (1976), which is hereby incorporated by reference in its entirety). This immortal cell line, which is preferably murine, but may also be derived from cells of other mammalian species, including without limitation, rats and humans, is selected to be deficient in enzymes necessary for the utilization of certain nutrients, to be capable of rapid growth, and have good fusion capability. Many such cell lines are known to those skilled in the art, and others are regularly described. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, “Continuous Cultures of Fused Cells Secreting Antibody of Predefined Specificity,” Nature 256:495 (1975), which is hereby incorporated by reference in its entirety.

Alternatively monoclonal antibodies can also be made using recombinant DNA methods as described in U.S. Pat. No. 4,816,567 to Cabilly et al, which is hereby incorporated by reference in its entirety. The polynucleotides encoding a monoclonal antibody are isolated from mature B-cells or hybridoma cells, for example, by RT-PCR using oligonucleotide primers that specifically amplify the genes encoding the heavy and light chains of the antibody. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells. Also, recombinant monoclonal antibodies or fragments thereof of the desired species can be isolated from phage display libraries as described (McCafferty et al., “Phage Antibodies: Filamentous Phage Displaying Antibody Variable Domains,” Nature 348:552-554 (1990); Clackson et al., “Making Antibody Fragments using Phage Display Libraries,” Nature 352:624-628 (1991); and Marks et al., “By-Passing Immunization. Human Antibodies from V-Gene Libraries Displayed on Phage,”J. Mol. Biol. 222:581-597 (1991), which are hereby incorporated by reference in their entirety).

The polynucleotide(s) encoding a monoclonal antibody can further be modified in a number of different manners using recombinant DNA technology to generate alternative antibodies. In one embodiment, the constant domains of the light and heavy chains of, for example, a mouse monoclonal antibody can be substituted for those regions of a human antibody to generate a chimeric antibody. Alternatively, the constant domains of the light and heavy chains of a mouse monoclonal antibody can be substituted for a non-immunoglobulin polypeptide to generate a fusion antibody. In other embodiments, the constant regions are truncated or removed to generate the desired antibody fragment of a monoclonal antibody. Furthermore, site-directed or high-density mutagenesis of the variable region can be used to optimize specificity and affinity of a monoclonal antibody.

In one embodiment of the present invention, the monoclonal antibody directed against the Ostβ or Ostα transmembrane domain region is a humanized antibody. Humanized antibodies are antibodies that contain minimal sequences from non-human (e.g. murine) antibodies within the variable regions. Such antibodies are used therapeutically to reduce antigenicity and human anti-mouse antibody responses when administered to a human subject. In practice, humanized antibodies are typically human antibodies with minimum to no non-human sequences. A human antibody is an antibody produced by a human or an antibody having an amino acid sequence corresponding to an antibody produced by a human.

An antibody can be humanized by substituting the complementarity determining region (CDR) of a human antibody with that of a non-human antibody (e.g. mouse, rat, rabbit, hamster, etc.) having the desired specificity, affinity, and capability (Jones et al., “Replacing the Complementarity-Determining Regions in a Human Antibody With Those From a Mouse,” Nature 321:522-525 (1986); Riechmann et al., “Reshaping Human Antibodies for Therapy,” Nature 332:323-327 (1988); Verhoeyen et al., “Reshaping Human Antibodies: Grafting an Antilysozyme Activity,” Science 239:1534-1536 (1988), which are hereby incorporated by reference in their entirety). The humanized antibody can be further modified by the substitution of additional residues either in the Fv framework region and/or within the replaced non-human residues to refine and optimize antibody specificity, affinity, and/or capability.

Human antibodies can be produced using various techniques known in the art. Immortalized human B lymphocytes immunized in vitro or isolated from an immunized individual that produce an antibody directed against a target antigen can be generated (See e.g. Reisfeld et al., Monoclonal Antibodies and Cancer Therapy 77 (Alan R. Liss 1985) and U.S. Pat. No. 5,750,373 to Garrard, which are hereby incorporated by reference in their entirety). Also, the human antibody can be selected from a phage library, where that phage library expresses human antibodies (Vaughan et al., “Human Antibodies with Sub-Nanomolar Affinities Isolated from a Large Non-immunized Phage Display Library,” Nature Biotechnology, 14:309-314 (1996); Sheets et al., “Efficient Construction of a Large Nonimmune Phage Antibody Library: The Production of High-Affinity Human Single-Chain Antibodies to Protein Antigens,” Proc. Nat'l. Acad. Sci. U.S.A. 95:6157-6162 (1998); Hoogenboom et al., “By-passing Immunisation. Human Antibodies From Synthetic Repertoires of Germline VII Gene Segments Rearranged In Vitro,” J. Mol. Biol. 227:381-8 (1992); Marks et al., “By-passing Immunization. Human Antibodies from V-gene Libraries Displayed on Phage,” J. Mol. Biol. 222:581-97 (1991), which are hereby incorporated by reference in their entirety). Humanized antibodies can also be made in transgenic mice containing human immunoglobulin loci that are capable upon immunization of producing the full repertoire of human antibodies in the absence of endogenous immunoglobulin production. This approach is described in U.S. Pat. Nos. 5,545,807 to Surani et al.; 5,545,806 to Lonberg et al.; 5,569,825 to Lonberg et al.; 5,625,126 to Lonberg et al.; 5,633,425 to Lonberg et al.; and 5,661,016 to Lonberg et al., which are hereby incorporated by reference in their entirety

Procedures for raising polyclonal antibodies are also well known. Typically, such antibodies can be raised by administering the peptide or polypeptide containing the epitope of interest (i.e. Ostα or Ostβ transmembrane domain peptide) subcutaneously to New Zealand white rabbits which have first been bled to obtain pre-immune serum. The antigens can be injected in combination with a synthetic surfactant adjuvant pluronic polyols, or pulverized acrylamide gel containing the protein or polypeptide after SDS-polyacrylamide gel electrophoresis. The rabbits are then bled approximately every two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. Ultimately, the rabbits are euthanized with pentobarbital 150 mg/Kg IV. This and other procedures for raising polyclonal antibodies are disclosed in Harlow et al., Eds., Antibodies: A Laboratory Manual, Cold Spring Harbor Laboratory, New York (1988), which is hereby incorporated by reference in its entirety.

In addition to whole antibodies, the present invention encompasses binding portions of such antibodies. Such binding portions include the monovalent Fab fragments, Fv fragments (e.g., single-chain antibody, scFv), and single variable V_{H} and V_{L} domains, and the bivalent F(ab′)₂ fragments, Bis-scFv, diabodies, triabodies, minibodies, etc. These antibody fragments can be made by conventional procedures, such as proteolytic fragmentation procedures, as described in J. Goding, Monoclonal Antibodies: Principles and Practice, (pp. 98-118) Academic Press: New York (1983), and Harlow et al., Antibodies: A Laboratory Manual, Cold Spring Harbor Laboratory, New York (1988), which are hereby incorporated by reference in their entirety, or other methods known in the art.

Particularly suitable for use in the present invention are antibody fragments engineered to bind to intracellular proteins, i.e. intrabodies. Intrabodies are generally obtained by selecting a single variable domain from variable regions of an antibody having two variable domains (i.e., a heterodimer of a heavy chain variable domain and a light chain variable domain). Methods for obtaining heavy chain-light chain heterodimers are described by Kohler et al., “Continuous Cultures of Fused Cells Secreting Antibody of Predefined Specificity,” Nature 256:495-7 (1975); Campbell et al., “Monoclonal Antibody Technology the Production and Characterization of Rodent and Human Hybridomas,” Burdon et al., Eds. Laboratory Techniques in Biochemistry and Molecular Biology, v. 13 Elsevier Science Publishers, Amsterdam (1985), which are hereby incorporated by reference in their entirety. Single chain Fv fragments which are the minimal recombinant antigen-binding fragments of antibodies are the most commonly used format for intrabody development. However, other suitable formats include Fab fragments, ScFv-Ck fusion proteins, single chain diabodies, V_{H}-C_{H}1 fragments, and even whole IgG molecules (Kontermann, R. E., “Intrabodies as Therapeutic Agents,” Methods 34:163-70 (2004), which is here by incorporated by reference in its entirety).

Intrabodies can be bi- or multi-functional. In addition to the antigen binding region, intrabodies can include other polypeptide regions defining a bioactive or function domain. Suitable functional domains include an enzyme, e.g., a protease, which can lead to the proteolysis of the Ostα or Ostβ protein. In another embodiment, the intrabody includes a targeting signal, e.g., ubiquitin, which can target Ostα or Ostβ to a proteasome for subsequent destruction. In yet another embodiment, the intrabody includes a targeting signal that is capable of retargeting the intrabody bound Ostα or Ostβ to another cellular locale. Such a locale may be cytoplasmic, nuclear, lysosomal, plasma membrane-associated, endoplasmic reticulum-associated, peroxisomal, or proteasomal. In addition, the intrabodies or binding molecules of the invention may encompass any art recognized targeting signal for altering the cellular location of a heterologous polypeptide.

Suitable intrabodies recognizing a transmembrane domain region of Ostα or Ostβ can be obtained from phage display, yeast surface display, or ribosome surface display. Methods for producing libraries of intrabodies and isolating intrabodies of interest are further described in U.S. Published Patent Application No. 20030104402 to Zauderer and U.S. Published Patent Application No. 20050276800 to Rabbitts, which are hereby incorporated by reference in their entirety. Methods for improving the stability and affinity binding characteristics of intrabodies are described in WO/2008/070363 to Zhenping and Contreras-Martinez et al., “Intracellular Ribosome Display via SecM Translation Arrest as a Selection for Antibodies with Enhanced Cytosolic Stability,” J. Mol. Biol. 372(2):513-24 (2007), which are hereby incorporated by reference in their entirety.

It may further be desirable, especially in the case of antibody fragments, to modify the antibody in order to increase its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

A number of antibody mimics are known in the art including, without limitation, those known as monobodies, which are derived from the tenth human fibronectin type III domain (¹⁰Fn3) (Koide et al., “The Fibronectin Type III Domain as a Scaffold for Novel Binding Proteins,”J. Mol. Biol. 284:1141-1151 (1998); Koide et al., “Probing Protein Conformational Changes in Living Cells by Using Designer Binding Proteins: Application to the Estrogen Receptor,” Proc. Natl. Acad. Sci. USA 99:1253-1258 (2002), each of which is hereby incorporated by reference in its entirety); and those known as affibodies, which are derived from the stable alpha-helical bacterial receptor domain Z of staphylococcal protein A (Nord et al., “Binding Proteins Selected from Combinatorial Libraries of an alpha-helical Bacterial Receptor Domain,” Nature Biotechnol. 15(8):772-777 (1997), which is hereby incorporated by reference in its entirety). Variations in the antibody mimics can be created by substituting one or more domains of these polypeptides and then screening the modified monobodies or affibodies for selective binding to one of the above-identified Ostα or Ostβ regions or domains.

Exemplary therapeutic agents of the present invention further include nucleic acid molecules that prevent Ostα and Ostβ complex formation by inhibiting

Ostα or Ostβ translation, binding to an Ostα or Ostβ transmembrane region, or encoding an inhibitory peptide or antibody that interferes with complex formation.

Nucleic acid molecules that prevent Ostα and Ostβ complex formation by inhibiting Ostα or Ostβ translation, i.e. inhibitory RNAs, include antisense RNAs or RNAi, such as short interfering RNAs (siRNA), short hairpin RNAs (shRNA), and microinterfering RNAs (miRNA).

The use of antisense methods to inhibit the in vivo translation of genes and subsequent protein expression is well known in the art (e.g., U.S. Pat. No. 7,425,544 to Dobie et al.; U.S. Pat. No. 7,307,069 to Karras et al.; U.S. Pat. No. 7,288,530 to Bennett et al.; U.S. Pat. No. 71,791,796 to Cowsert et al., which are hereby incorporated by reference in their entirety). Antisense nucleic acids are nucleic acid molecules (e.g., molecules containing DNA nucleotides, RNA nucleotides, or modifications (e.g., modification that increase the stability of the molecule, such as 2′-O-alkyl (e.g., methyl) substituted nucleotides) or combinations thereof) that are complementary to, or that hybridize to, at least a portion of a specific nucleic acid molecule, such as an mRNA molecule (see, e.g., Weintraub, H. M., “Antisense DNA and RNA,” Scientific Am. 262:40-46 (1990), which is hereby incorporated by reference in its entirety). The antisense nucleic acids hybridize to corresponding nucleic acids, such as mRNAs, to form a double-stranded molecule, which interferes with translation of the mRNA, as the cell will not translate a double-stranded mRNA. Antisense nucleic acids used in the methods of the present invention are typically at least 10-12 nucleotides in length, for example, at least 15,20,25,50, 75, or 100 nucleotides in length. The antisense nucleic acid can also be as long as the target nucleic acid with which it is intended to form an inhibitory duplex. Antisense nucleic acids can be introduced into cells as antisense oligonucleotides, or can be produced in a cell in which a nucleic acid encoding the antisense nucleic acid has been introduced, for example, using gene therapy methods.

siRNAs are double stranded synthetic RNA molecules approximately 20-25 nucleotides in length with short 2-3 nucleotide 3′ overhangs on both ends. The double stranded siRNA molecule represents the sense and anti-sense strand of a portion of the target mRNA molecule, in this case a portion of the Ostα or Ostβ mRNA sequence. Numerous reports have been published on critical advances in the understanding of the biochemistry and genetics of both gene silencing and RNAi (Matzke et al., “RNA-Based Silencing Strategies in Plants,” Curr. Opin. Genet. Dev. 11(2):221-227 (2001), which is hereby incorporated by reference in its entirety). In RNAi, the introduction of double stranded RNA (dsRNA, or iRNA, for interfering RNA) into animal or plant cells leads to the destruction of the endogenous, homologous mRNA, phenocopying a null mutant for that specific gene. In both post-transcriptional gene silencing and RNAi, the dsRNA is processed to short interfering molecules of 21-, 22-, or 23-nucleotide RNAs (siRNA) by a putative RNAaseIII-like enzyme (Tuschl T., “RNA Interference and Small Interfering RNAs,” Chembiochem 2:239-245 (2001); Zamore et al., “RNAi: Double Stranded RNA Directs the ATP-Dependent Cleavage of mRNA at 21 to 23 Nucleotide Intervals,” Cell 101:25-3, (2000), each of which is hereby incorporated by reference in its entirety). The endogenously generated siRNAs mediate and direct the specific degradation of the target mRNA. In the case of RNAi, the cleavage site in the mRNA molecule targeted for degradation is located near the center of the region covered by the siRNA (Elbashir et al., “RNA Interference is Mediated by 21- and 22-Nucleotide RNAs,” Gene Dev. 15(2):188-200 (2001), which is hereby incorporated by reference in its entirety). The dsRNA for Ostα or Ostβ can be generated by transcription in vivo, which involves modifying the nucleic acid molecule encoding Ostα or Ostβ for the production of dsRNA, inserting the modified nucleic acid molecule into a suitable expression vector having the appropriate 5′ and 3′ regulatory nucleotide sequences operably linked for transcription and translation, and introducing the expression vector having the modified nucleic acid molecule into a suitable host cell or subject. Alternatively, complementary sense and antisense RNAs derived from a substantial portion of the coding region of the Ostα or Ostβ nucleic acid molecule are synthesized in vitro (Fire et al., “Specific Interference by Ingested dsRNA,” Nature 391:806-811 (1998); Montgomery et al, “RNA as a Target of Double-Stranded RNA-Mediated Genetic Interference in Caenorhabditis elegans,” Proc Natl Acad Sci USA 95:15502-15507; Tabara et al., “RNAi in C. elegans: Soaking in the Genome Sequence,” Science 282:430-431 (1998), each of which is hereby incorporated by reference in its entirety). The resulting sense and antisense RNAs are annealed in an injection buffer, and dsRNA is administered to the subject using any method of administration described herein.

siRNA molecules are typically designed to target a region of the mRNA target approximately 50-100 nucleotides downstream from the start codon. siRNA for Ostα or Ostβ can be selected using the online services provided by GenScript, Promega, or Ambion Inc. Various improvements of siRNA compositions, such as the incorporation of modified nucleosides or motifs into one or both strands of the siRNA molecule to enhance stability, specificity, and efficacy, have been described and are suitable for use in accordance with this aspect of the invention (See e.g., WO 2004/015107 to Giese et al.; WO 2003/070918 to McSwiggen et al.; WO 1998/39352 to Imanishi et al.; U.S. Patent Application Publication No. 2002/0068708 to Jesper et al.; U.S. Patent Application Publication No. 2002/0147332 to Kaneko et al; U.S. Patent Application Publication No. 2008/0119427 to Bhat et al., which are hereby incorporated by reference in their entirety). Ostα and Ostβ siRNA are commercially available from Santa Cruz Biotechnology.

Short or small hairpin RNA molecules are similar to siRNA molecules in function, but comprise longer RNA sequences that make a tight hairpin turn. shRNA is cleaved by cellular machinery into siRNA, which silences gene expression via the cellular RNA interference pathway. Ostα and Ostβ shRNA are commercially available from Santa Cruz Biotechnology and OriGene Technologies.

Suitable therapeutic nucleic acid molecules of the present invention further include aptamers which bind to at least a portion of the Ostβ transmembrane domain that is illustrated in FIG. 2B (i.e. SEQ ID NOs: 87-96) or to at least a portion of one of the Ostα transmembrane domains that are illustrated in FIGS. 1A-C (SEQ ID NOs: 17-86).

Aptamers are single-stranded, partially single-stranded, partially double-stranded, or double-stranded nucleotide sequences, advantageously a replicatable nucleotide sequence, capable of specifically recognizing a selected nonoligonucleotide molecule or group of molecules by a mechanism other than Watson-Crick base pairing or triplex formation. Aptamers include, without limitation, defined sequence segments and sequences comprising nucleotides, ribonucleotides, deoxyribonucleotides, nucleotide analogs, modified nucleotides and nucleotides comprising backbone modifications, branchpoints and normucleotide residues, groups or bridges. Aptamers include partially and fully single-stranded and double-stranded nucleotide molecules and sequences; synthetic RNA, DNA, and chimeric nucleotides; hybrids; duplexes; heteroduplexes; and any ribonucleotide, deoxyribonucleotide, or chimeric counterpart thereof and/or corresponding complementary sequence, promoter, or primer-annealing sequence needed to amplify, transcribe, or replicate all or part of the aptamer molecule or sequence.

Nucleic acid aptamers include multivalent aptamers and bivalent aptamers. Methods of making bivalent and multivalent aptamers and their expression in multi-cellular organisms are described in U.S. Pat. No. 6,458,559 to Shi et al., which is hereby incorporated by reference in its entirety. A method for modular design and construction of multivalent nucleic acid aptamers, their expression, and methods of use are described in U.S. Patent Publication No. 2005/0282190, which is hereby incorporated by reference in its entirety. Aptamers may be designed to inhibit translation of Ostα or Ostβ, inhibit heteromeric complex formation, and/or to bind to and inhibit transport activity of an otherwise functional Ostα-Ostβ transporter.

Identifying suitable nucleic acid aptamers of the present invention that inhibit translation of Ostα or Ostβ basically involves selecting aptamers that bind Ostα or Ostβ mRNA or protein with sufficiently high affinity (e.g., K_{d}=20−50 nM) and specificity from a pool of nucleic acids containing a random region of varying or predetermined length (Shi et al., “A Specific RNA Hairpin Loop Structure Binds the RNA Recognition Motifs of the Drosophila SR Protein B52,” Mol. Cell. Biol. 17:1649-1657 (1997), which is hereby incorporated by reference in its entirety).

Using these same procedures, suitable nucleic acid aptamers of the present invention that inhibit heteromeric complex formation or inhibit activity of the Ost transporter can also be identified. Rather than screening aptamer pools against the mRNA, instead Ostα or Ostβ peptide domains can be used to select and enrich the aptamers.

For example, identifying suitable nucleic acid aptamers can be carried out using an established in vitro selection and amplification scheme known as SELEX. The SELEX scheme is described in detail in U.S. Pat. No. 5,270,163 to Gold et al.; Ellington and Szostak, “In Vitro Selection of RNA Molecules that Bind Specific Ligands,” Nature 346:818-822 (1990); and Tuerk & Gold, “Systematic Evolution of Ligands by Exponential Enrichment: RNA Ligands to Bacteriophage T4 DNA Polymerase,” Science 249:505-510 (1990), which are hereby incorporated by reference in their entirety. The SELEX procedure can be modified so that an entire pool of aptamers with binding affinity can be identified by selectively partitioning the pool of aptamers. This procedure is described in U.S. Patent Application Publication No. 20040053310, which is hereby incorporated by reference in its entirety. Aptamers that bind to and inhibit activity of the Ostα-Ostβ transporter may also be identified using screening assays such as yeast-two hybrid approaches described in U.S. Patent Application Serial No. 20040210040 to Landolfi et al., which is hereby incorporated by reference in its entirety.

A fourth aspect of the present invention is directed to a method of treating a condition associated with altered (i.e., a disruption in) bile acid homeostasis in a patient. This method includes providing a therapeutic agent that modulates Ostα-Ostβ heteromeric complex formation or modulates Ostα-Ostβ transporter activity, and administering the therapeutic agent to modulate bile acid in the patient. Administration of the therapeutic agent is effective, to modulate bile acid transport, to treat the condition associated with altered bile acid homeostasis in the patient.

As used herein, therapeutic agents that “modulate” Ostα-Ostβ heteromeric complex formation or functional transport activity include agents that enhance heteromeric complex formation or transport activity to increase bile acid transport as well as agents that inhibit complex formation or functional transport activity to decrease bile acid transport. Therapeutic agents suitable for inhibiting Ostα-Ostβ complex formation and/or transport activity include the inhibitory peptides, antibodies, and nucleic acid molecules described supra. Therapeutic agents suitable for enhancing Ostα-Ostβ transport activity are those which enhance Ostα and Ostβ expression. These agents include nucleic acid molecules encoding Ostα (e.g. SEQ ID NOs: 2, 4, 6, and 8) and Ostβ (e.g. SEQ ID NOs: 10, 12, 14, 16) proteins. In a preferred embodiment, the nucleic acid molecules encoding Ostα or Ostβ are incorporated into an expression vector. The expression vector will preferably contain a tissue-specific promoter to direct expression of the nucleic acid molecule(s) encoding Ostα and/or Ostβ to a specific tissue or cell type. Suitable tissue-specific promoters are described infra and include those that direct expression to the kidney, liver, or small intestine

In accordance with this aspect of the present invention, conditions associated with altered bile acid homeostasis include, without limitation, bile acid malabsorption, cholestasis, cholelithiasis, bile acid induced oxidative stress, necrotizing enterocolitis, and colon cancer.

Bile acid malabsorption involves a disruption in bile acid transport from the ileum lumen to the portal circulation, and as a result not enough bile acid is reabsorbed. Therefore, suitable therapeutic agents for treating bile acid malabsorption include those agents that enhance Ostα-Ostβ bile acid transport. Bile acid malabsorption is observed in a number of human conditions, including intractable diarrhea, irritable bowel syndrome, immunodeficiency virus (HIV) enteropathy, and cystic fibrosis (Weber et al., “Malabsorption of Bile Acids in Children With Cystic Fibrosis,” N. Engl. J. Med. 289:1001-1005 (1973); O'Brien et al., “Intestinal Bile Acid Malabsorption in Cystic Fibrosis,” Gut 34:1137-1141 (1993); Sciarretta et al., “Bile Acid Malabsorption in AIDS-Associated Chronic Diarrhea: A Prospective 1-Year Study,” Am. J. Gastroenterol. 89:379-381 (1994); and Steuerwald et al., “HIV-Enteropathy and Bile Acid Malabsorption: Response to Cholestyramine,”Am. Gastroenterol. 90:2051-2053 (1995), which are hereby incorporated by reference in their entirety), all of which are suitable for treatment in accordance with this aspect of the present invention.

Excessive bile acid reabsorption or transport across the enterocytes of the ileum causes constipation (van Tilburg et al. “Na⁺-Dependent Bile Acid Transport in The Ileum: The Balance Between Diarrhea and Constipation,” Gastroenterology 98:25-32 (1990), which is hereby incorporated by reference in its entirety). Accordingly, suitable therapeutic agents for treating this type of constipation include those agents which inhibit Ostα-Ostβ complex formation and transport activity, including any of those described supra.

Cholelithiasis is another condition associated with altered bile acid homeostasis and altered lipid levels. In general, cholesterol gallstones develop when bile contains too much cholesterol and not enough bile salts, and thus enhanced bile acid absorption from the intestine might provide a benefit for such individuals, if carried out in conjunction with a low cholesterol diet. Therefore, suitable therapeutic agents for treating cholelithiasis include those agents that enhance Ostα-Ostβ bile acid transport.

Other conditions leading to elevated, and often toxic bile acid concentrations, where inhibition of Ostα-Ostβ transport activity is desired, include bile acid induced oxidative stress, with secondary consequences of inflammation and fibrosis, necrotizing enterocolitis (NEC), and colon cancer. Suitable therapeutic agents for treating these conditions include agents that inhibit Ostα-Ostβ bile acid transport.

Cholestasis is another condition associated with altered bile acid homeostasis that is suitable for treatment in accordance with this aspect of the present invention. Cholestasis is a condition where bile transport from the liver to the duodenum is disrupted or blocked. Decreasing bile transport via the Ostα-Ostβ transporter should diminish liver injury associated with cholestatic disease. Therefore, suitable therapeutic agents for treating cholestasis and cholestatic liver diseases, include those agents that block Ostα-Ostβ heterocomplex formation or Ostα-Ostβ transport activity. In one preferred embodiment, the therapeutic agent which blocks Ostα-Ostβ heterocomplex formation and/or transport activity includes a tissue specific targeting component to achieve inhibition of Ostα-Ostβ complex formation or transport activity in the proximal tubule of the kidney, thereby facilitating increased renal excretion of bile acids. In another preferred embodiment, the therapeutic agent which blocks Ostα-Ostβ heterocomplex formation and/or transport activity includes a tissue specific targeting component to achieve inhibition of Ostα-Ostβ complex formation or transport activity in hepatocytes, which should reduce total bile acid secretion. Suitable strategies for achieving tissue-specific delivery of the therapeutic agents of the present invention are described infra.

From the foregoing, it should thus be appreciated that a further aspect of the present invention relates to an isolated nucleic acid or polypeptide that binds to either (i) an Ostα or an Ostβ transmembrane domain region, which upon binding prevents formation of an Ostα-Ostβ heteromeric complex, or (ii) an Ostα transport domain, which upon binding prevents transport by the Ostα-Ostβ transporter complex. The present invention also relates to isolated nucleic acid molecules that encode a functional Ostα or an Ostβ protein. Suitable nucleic acids and polypeptides are described above.

A further aspect of the invention relates to a pharmaceutical composition that includes an isolated nucleic acid, peptide, antibody, or small molecule of the present invention and a pharmaceutically acceptable carrier.

The isolated nucleic acid of the pharmaceutical composition (i.e. inhibitory RNA molecule targeting Ostα or Ostβ, a nucleic acid molecule encoding the inhibitory Ostα or Ostβ peptides or antibodies, or a nucleic acid molecule encoding the Ostα or Ostβ protein) will preferably be contained in a gene therapy vector to facilitate delivery of the nucleic acid molecule to the target tissue and, if appropriate, in vivo expression of the Ostα or Ostβ peptide, antibody, or protein. Delivery and expression thereof of an Ostα or Ostβ inhibitory peptide or antibody can prevent the formation of an active Ostα-Ostβ transporter. Alternatively, delivery and expression of nucleic acids encoding the Ostα and Ostβ proteins will enhance transporter expression and, subsequently, activity. Thus, naked DNA or infective transformation vectors can be used for delivery, whereby the naked DNA or infective transformation vector contains a recombinant gene that encodes the peptide, antibody, protein, or RNA. The peptide, antibody, or RNA molecule is then expressed in the transformed cell, and inhibits or enhances the formation of an active Ostα-Ostβ transporter.

The recombinant gene includes, operatively coupled to one another, an upstream promoter operable in mammalian cells and optionally other suitable regulatory elements (i.e., enhancer or inducer elements), a coding sequence that encodes the therapeutic nucleic acid or peptide (described above), and a downstream transcription termination region. Any suitable constitutive promoter or inducible promoter can be used to regulate transcription of the recombinant gene, and one of skill in the art can readily select and utilize such promoters, whether now known or hereafter developed. In accordance with the various methods of the present invention it may be desirable to incorporate a promoter that directs tissue or cell specific expression of the recombinant nucleic acid to the tissues and cells expressing Ostα and Ostβ. Preferably, such tissue specific promoter would direct expression to epithelial tissue of the intestine (e.g. enterocytes), kidney, liver (e.g. hepatocytes), or bile duct (e.g. cholangiocytes). An exemplary enterocyte-specific promoter includes, without limitation, the sucrose isomaltase promoter. Exemplary kidney-specific promoters include, without limitation, ksp-cadherin promoter, uromodulin promoter, and chloride channel K1 promoter. Exemplary hepatocyte-specific promoters include without limitation, transthyretin promoter, cytochrome P450 2C6 promoter, α₁-antitrypsin promoter carrying an apoE enhancer, and albumin promoter. An exemplary cholangiocyte-specific promoter includes, without limitation, cytokeratin 19 promoter. The utilization of tissue or cell specific promoters is particularly desirable when trying to enhance Ostα and Ostβ expression as a strategy for enhancing bile transport. Tissue specific promoters can also be made inducible/repressible using, e.g., a TetO response element. Other inducible elements can also be used. Known recombinant techniques can be utilized to prepare the recombinant gene, transfer it into the expression vector (if used), and administer the vector or naked DNA to a patient. Exemplary procedures are described in Sambrook and Russell, Molecular Cloning: A Laboratory Manual (2d ed. 1989), which is hereby incorporated by reference in its entirety. One of skill in the art can readily modify these procedures, as desired, using known variations of the procedures described therein.

Any suitable viral or infective transformation vector can be used to deliver a transgene. Exemplary viral vectors include, without limitation, adenovirus, adeno-associated virus, and retroviral vectors (including lentiviral vectors).

Adenovirus gene delivery vehicles can be readily prepared and utilized given the disclosure provided in Berkner, “Development of Adenovirus Vectors for the Expression of Heterologous Genes,” Biotechniques 6:616-29 (1988); Rosenfeld et al., “Adenovirus-mediated Transfer of a Recombinant α 1-Antitrypsin Gene to the Lung Epithelium in Vivo,” Science 252:431-434 (1991); International Patent Publication No. WO 1993/007283 to Curiel et al.; International Patent Publication No. WO 1993/006223 to Perricaudet et al.; and International Patent Publication No. WO 1993/007282 to Curiel et al., which are hereby incorporated by reference in their entirety. Additional types of adenovirus vectors are described in U.S. Pat. No. 6,057,155 to Wickham et al., U.S. Pat. No. 6,033,908 to Bout & Hoeben, U.S. Pat. No. 6,001,557 to Wilson et al., U.S. Pat. No. 5,994,132 to Chamberlain & Kumar-Singh, U.S. Pat. No. 5,981,225 to Kochanek & Schniedner, U.S. Pat. No. 5,885,808 to Spooner & Epenetos, and U.S. Pat. No. 5,871,727 to Curiel, which are hereby incorporated by reference in their entirety.

Adeno-associated viral gene delivery vehicles can be constructed and used to deliver into cells a recombinant gene encoding a desired nucleic acid. The use of adeno-associated viral gene delivery vehicles in vitro is described in Chatterjee et al., “Dual-Target Inhibition of HIV-1 in Vitro by Means of an Adeno-associated Virus Antisense Vector,” Science 258:1485-8 (1992); Walsh et al., “Regulated High Level Expression of a Human γ-Globin Gene Introduced into Erythroid Cells by an Adeno-associated Virus Vector,” Proc. Nat'l Acad. Sci. USA 89:7257-61 (1992); Walsh et al., “Phenotypic Correction of Fanconi Anemia in Human Hematopoietic Cells with a Recombinant Adeno-associated Virus Vector,” J. Clin. Invest. 94:1440-8 (1994); Flotte et al., “Expression of the Cystic Fibrosis Transmembrane Conductance Regulator from a Novel Adeno-associated Virus Promoter,”J. Biol. Chem. 268:3781-90 (1993); Ponnazhagan et al., “Suppression of Human α-Globin Gene Expression Mediated by the Recombinant Adeno-associated Virus 2-based Antisense Vectors,” J. Exp. Med. 179:733-8 (1994); Miller et al., “Recombinant Adeno-associated Virus (rAAV)-mediated Expression of a Human γ-Globin Gene in Human Progenitor-derived Erythroid Cells,” Proc. Nat'l Acad. Sci. USA 91:10183-7 (1994); Einerhand et al., “Regulated High-level Human β-Globin Gene Expression in Erythroid Cells Following Recombinant Adeno-associated Virus-mediated Gene Transfer,” Gene Ther. 2:336-43 (1995); Luo et al., “Adeno-associated Virus 2-mediated Gene Transfer and Functional Expression of the Human Granulocyte-macrophage Colony-stimulating Factor,” Exp. Hematol. 23:1261-7 (1995); and Zhou et al., “Adeno-associated Virus 2-mediated Transduction and Erythroid Cell-specific Expression of a Human β-Globin Gene,” Gene Ther. 3:223-9 (1996), which are hereby incorporated by reference in their entirety. In vivo use of these vehicles is described in Flotte et al., “Stable in Vivo Expression of the Cystic Fibrosis Transmembrane Conductance Regulator with an Adeno-associated Virus Vector,”Proc. Nat'l Acad. Sci. USA 90:10613-7 (1993), and Kaplitt et al., “Long-term Gene Expression and Phenotypic Correction Using Adeno-associated Virus Vectors in the Mammalian Brain,” Nat. Genet. 8:148-54 (1994), which are hereby incorporated by reference in their entirety.

Retroviral vectors which have been modified to form infective transformation systems can also be used to deliver a recombinant gene encoding a desired nucleic acid product into a target cell. One such type of retroviral vector is disclosed in U.S. Pat. No. 5,849,586 to Kriegler & Perez, which is hereby incorporated by reference in its entirety. Lentivirus vectors can also be utilized, including those described in U.S. Pat. No. 6,790,657 to Arya, U.S. Patent Application Publication No. 2004/0170962 to Kafri et al., and U.S. Patent Application Publication No. 2004/0147026 to Arya, which are hereby incorporated by reference in their entirety.

Alternatively, the nucleic acid of the pharmaceutical composition, in particular the inhibitory RNA nucleic acids, can be formulated as a component of a composition. Suitable compositions include the siRNA formulated or complexed with polyethylenimine (e.g., linear or branched PEI) and/or polyethylenimine derivatives, including for example grafted PEIs such as galactose PEI, cholesterol PEI, antibody derivatized PEI, and polyethylene glycol PEI derivatives thereof (see, e.g., Blazek-Welsh & Rhodes, “Maltodextrin-based Proniosomes,”AAPS Pharm. Sci. 3(1):1-11 (2001); Furgeson et al., “Modified linear polyethylenimine-cholesterol conjugates for DNA complexation,” Bioconjug. Chem. 14:840-7 (2003); Kunath et al., “The structure of PEG-modified poly(ethylene imines) influences biodistribution and pharmacokinetics of their complexes with NF-κB decoy in mice,” Pharm. Res. 19:810-7 (2002); Choi et al., “Effect of Poly(ethylene glycol) Grafting on Polyethylenimine as a Gene Transfer Vector in Vitro,” Bull. Korean Chem. Soc. 22(1):46-52 (2001); Bettinger et al., “Size Reduction of Galactosylated PEI/DNA Complexes Improves Lectin-mediated Gene Transfer into Hepatocytes,” Bioconjug. Chem. 10:558-61 (1999); Petersen et al., “Polyethylenimine-graft-poly(ethylene glycol) copolymers: influence of copolymer block structure on DNA complexation and biological activities as gene delivery system,” Bioconjug. Chem. 13:845-54 (2002); Erbacher et al., “Transfection and physical properties of various saccharide, poly(ethylene glycol), and antibody-derivatized polyethylenimines (PEI),” J. Gene Med. 1(3):210-22 (1999); Godbey et al., “Tracking the intracellular path of poly(ethylenimine)/DNA complexes for gene delivery,” Proc. Nat'l Acad. Sci. USA 96:5177-81 (1999); Godbey et al., “Poly(ethylenimine) and its role in gene delivery,” J. Control. Release 60:149-60 (1999); Diebold et al., “Mannose Polyethylenimine conjugates for targeted DNA delivery into dendritic cells,” J. Biol. Chem. 274:19087-94 (1999); Thomas & Klibanov, “Enhancing polyethylenimine's delivery of plasmid DNA into mammalian cells,” Proc. Nat'l Acad. Sci. USA 99:14640-5 (2002); U.S. Pat. No. 6,586,524 to Sagara, which are hereby incorporated by reference in their entirety).

The inhibitory RNA molecule can also be present in the form of a bioconjugate, for example a nucleic acid conjugate as described in U.S. Pat. No. 6,528,631 to Cook et al., U.S. Pat. No. 6,335,434 to Guzaev et al., U.S. Pat. No. 6,235,886 to Manoharan & Cook, U.S. Pat. No. 6,153,737 to Manoharan et al., U.S. Pat. No. 5,214,136 to Lin & Matteucci, and U.S. Pat. No. 5,138,045 to Cook & Guinosso, which are hereby incorporated by reference in their entirety.

Pharmaceutical compositions of the present invention may contain any of the Ostα and Ostβ peptides or fragments thereof described supra. In a preferred embodiment of the present invention, the Ostα and Ostβ peptides of the pharmaceutical composition are modified, e.g. conjugated to a targeting component, as described infra or incorporated into an appropriate delivery vehicle, e.g. a liposome, to facilitate its delivery to a target cell, tissue, or organ.

One such modification involves conjugating the Ostα or Ostβ inhibitory peptide of interest to a peptide capable of targeting the peptide of interest to a particular tissue, cell, or compartment within the cell. In one embodiment, the targeting peptide is a ligand domain that is specific for receptors located on a target cell, such as the hepatocyte-specific asialoglycoprotein receptor. Thus, when the chimeric protein is delivered intravenously or otherwise introduced into blood or lymph, the chimeric protein will adsorb to the targeted cell, and the targeted cell will internalize the chimeric protein. Methods of preparing such chimeric proteins are described in U.S. Pat. No. 5,817,789 to Heartlein et al., which is hereby incorporated by reference in its entirety. Alternatively, the targeting peptide can be a cell penetrating peptide (CPP). CPPs translocate across the plasma membrane of eukaryotic cells by a seemingly energy-independent pathway and have been used successfully for intracellular delivery of macromolecules, including antibodies, peptides, proteins, and nucleic acids, with molecular weights several times greater than their own. Several commonly used CPPs, including polyarginines, transportant, protamine, maurocalcine, and M918, are suitable delivery vehicles for use in the present invention and are well known in the art (see Stewart et al., “Cell-Penetrating Peptides as Delivery Vehicles for Biology and Medicine,” Organic Biomolecular Chem 6:2242-2255 (2008), which is hereby incorporated by reference in its entirety). Additionally, methods of making CPP are described in U.S. Patent Application Publication No. 20080234183 to Hallbrink et al., which is hereby incorporated by reference in its entirety. The inhibitory peptides of interest can further possess a signal peptide that directs its compartmentalization once it is internalized. Preferably, the signal peptide is an endoplasmic reticulum (ER) signal peptide sequence. A number of such signal peptides are known in the art, including the signal peptide of SEQ ID NO: 138 (MMSFVSLLLVGILFYATEAEQLTKCEVFQ). Other suitable ER signal peptides include the N-terminus endoplasmic reticulum targeting sequence of the enzyme 17β-hydroxysteroid dehydrogenase type 11 (Horiguchi et al., “Identification and Characterization of the ER/Lipid Droplet-Targeting Sequence in 1713-hydroxysteroid Dehydrogenase Type 11,” Arch. Biochem. Biophys. 479(2):121-30 (2008), which is hereby incorporated by reference in its entirety), or any of the ER signaling peptides (including the nucleic acid sequences encoding the ER signal peptides) disclosed in U.S. Patent Publication No. 20080250515 to Reed et al., which is hereby incorporated by reference in its entirety. Additionally, the inhibitory peptide of the present invention can also be modified to include an ER retention signal, such as the retention signal of SEQ ID NO: 139 (KDEL). Methods of modifying the therapeutic agents of the present invention to incorporate signal and/or retentions peptides for localization and retention of the therapeutic agent in the ER are described in U.S. Patent Publication No. 20080250515 to Reed et al., which is hereby incorporated by reference in its entirety.

It may also be desirable to conjugate the Ostα or Ostβ inhibitory peptides to a polymer or small molecule that is stabilized to avoid enzymatic degradation of the conjugated protein or polypeptide. Conjugated proteins or polypeptides of this type are described in U.S. Pat. No. 5,681,811 to Ekwuribe, which is hereby incorporated by reference in its entirety.

The nucleic acids or peptides of the pharmaceutical composition may further be encapusulated or incorporated into an appropriate delivery vehicle. Suitable delivery vehicles include, but are in no way limited to, liposomes, nanoparticles, biodegradable microspheres, microparticles, and collagen minipellets.

Liposomes are vesicles comprised of one or more concentrically ordered lipid bilayers which encapsulate an aqueous phase. They are normally not leaky, but can become leaky if a hole or pore occurs in the membrane, if the membrane is dissolved or degrades, or if the membrane temperature is increased to the phase transition temperature. Current methods of drug delivery via liposomes require that the liposome carrier ultimately become permeable and release the encapsulated drug at the target site. This can be accomplished, for example, in a passive manner where the liposome bilayer degrades over time through the action of various agents in the body. Every liposome composition will have a characteristic half-life in the circulation or at other sites in the body and, thus, by controlling the half-life of the liposome composition, the rate at which the bilayer degrades can be somewhat regulated.

In contrast to passive drug release, active drug release involves using an agent to induce a permeability change in the liposome vesicle. Liposome membranes can be constructed so that they become destabilized when the environment becomes acidic near the liposome membrane (see, e.g., Wang & Huang, “pH-Sensitive Immunoliposomes Mediate Target-cell-specific Delivery and Controlled Expression of a Foreign Gene in Mouse,” Proc. Nat'l Acad. Sci. USA 84:7851-5 (1987), which is hereby incorporated by reference in its entirety). When liposomes are endocytosed by a target cell, for example, they can be routed to acidic endosomes which will destabilize the liposome and result in drug release.

This liposome delivery system can also be made to accumulate at a target organ, tissue, or cell via active targeting (e.g., by incorporating an antibody or hormone on the surface of the liposomal vehicle). This can be achieved according to known methods.

Different types of liposomes can be prepared according to Bangham et al., “Diffusion of Univalent Ions Across the Lamellae of Swollen Phospholipids,” J. Mol. Biol. 13:238-52 (1965); U.S. Pat. No. 5,653,996 to Hsu; U.S. Pat. No. 5,643,599 to Lee et al.; U.S. Pat. No. 5,885,613 to Holland et al.; U.S. Pat. No. 5,631,237 to Dzau & Kaneda; and U.S. Pat. No. 5,059,421 to Loughrey et al., which are hereby incorporated by reference in their entirety.

The pharmaceutical composition of the present invention also contains a carrier. Acceptable pharmaceutical carriers include solutions, suspensions, emulsions, excipients, powders, or stabilizers. The carrier should be suitable for the desired mode of delivery, discussed infra.

Pharmaceutical compositions suitable for injectable use (e.g., intravenous, intra-arterial, intramuscular, etc.) may include sterile aqueous solutions or dispersions and sterile powders for the extemporaneous preparation of sterile injectable solutions or dispersions. In all cases, the form should be sterile and should be fluid to the extent that easy syringability exists. It should be stable under the conditions of manufacture and storage and should be preserved against the contaminating action of microorganisms, such as bacteria and fungi. Suitable adjuvants, carriers and/or excipients, include, but are not limited to sterile liquids, such as water and oils, with or without the addition of a surfactant and other pharmaceutically and physiologically acceptable carrier. Illustrative oils are those of petroleum, animal, vegetable, or synthetic origin, for example, peanut oil, soybean oil, or mineral oil. In general, water, saline, aqueous dextrose and related sugar solutions, and glycols, such as propylene glycol or polyethylene glycol, are preferred liquid carriers, particularly for injectable solutions.

Oral dosage formulations of the pharmaceutical composition of the present invention can include standard carriers such as pharmaceutical grades of mannitol, lactose, starch, magnesium stearate, sodium saccharine, cellulose, magnesium carbonate, etc. Suitable carriers include lubricants and inert fillers such as lactose, sucrose, or cornstarch. In another embodiment, these compounds are tableted with conventional tablet bases such as lactose, sucrose, or cornstarch in combination with binders like acacia, gum gragacanth, cornstarch, or gelatin; disintegrating agents such as cornstarch, potato starch, or alginic acid; a lubricant like stearic acid or magnesium stearate; and sweetening agents such as sucrose, lactose, or saccharine; and flavoring agents such as peppermint oil, oil of wintergreen, or artificial flavorings. Generally, the ingredients are supplied either separately or mixed together in unit dosage form, for example, as a dry lyophilized powder or water free concentrate in a hermetically sealed container such as an ampule or sachette indicating the quantity of active agent.

As is known in the art, because orally administered agents need to survive the digestive system before cellular uptake, it is possible to administer the therapeutic agents of the present invention with any of a variety of stabilizing reagent that inhibits destruction thereof. One such type of stabilizing reagent is a mammalian colostrum, whether produced as a hyperimmune colostrum for antibody-based therapeutics or as an in vitro mixture of the therapeutic agent and colostrum.

The pharmaceutical composition of the preset invention can be administered by any means suitable for producing the desired therapeutic endpoint. Preferred delivery routes include orally, by inhalation, by intranasal instillation, topically, transdermally, parenterally, subcutaneously, intravenous injection, intra-arterial injection, intramuscular injection, intraplurally, intraperitoneally, or by application to mucous membrane. The composition can be delivered repeatedly over a course of time to achieve optimal therapeutic response.

Effective amounts of the pharmaceutical composition of the present invention will depend upon the mode of administration, frequency of administration, nature of the treatment, age and condition of the individual to be treated, and the type of pharmaceutical composition used to deliver the therapeutic agent. Effective levels of the composition may range from about 0.001 to about 2.5 mg/kg depending upon the clinical endpoints and toxicity thresholds. While individual doses may vary, optimal ranges of the effective amounts may be determined by one of ordinary skill in the art.

The pharmaceutical composition containing the therapeutic agent of the present invention can be administered to a patient alone or in combination with any other standard therapy to treat dyslipidemia and dyslipidemia related diseases, or in combination with any other standard therapy to treat conditions associated with or caused by altered bile acid homeostasis in a patient.

Finally, the present invention also relates to a non-human mammal having in its germ and somatic cells an artificially induced Ostα null mutation, wherein said mammal does not express Ostα protein. An exemplary Ostα−/− rodent of this type is described infra.

### EXAMPLES

The present invention is illustrated, but not limited, by the following examples.

### Material and Methods for Examples 1-6

Materials. HEK293 cells were obtained from the American Type Culture Collection (ATCC) (CRL-1573) and grown as monolayers at 37° C. in an atmosphere of 5% CO₂. Cells were maintained in medium consisting of DMEM (GIBCO, 10-013-CV) with 10% (v/v) fetal calf serum and antibiotics. Antibodies to mouse Ostα (mA315) and Ostβ (mB91) have been described previously (Dawson et al., “The Heteromeric Organic Solute Transporter α-β, Ostα-Ostβ, is an Ileal Basolateral Bile Acid Transporter,” J. Biol. Chem. 280:6960-6968 (2005); and Ballatori et al., “Ostα-Ostβ, A Major Basolateral Bile Acid and Steroid Transporter in Human Intestinal, Renal, and Biliary Epithelia,” Hepatology 42:1270-1279 (2005), which are hereby incorporated by reference in their entirety). The anti-FLAG M2 monoclonal (F 3165) and anti-c-myc clone 9E10 (M 5546) antibodies were purchased from Sigma-Aldrich, the anti-MRP1 antibody (ALX-210-841, which is known to cross-react with mouse Mrp1) was from Alexis Biochemicals and the anti-13 Actin (JLA20) was purchased from Calbiochem. PNGase F (P0704L) was purchase from New England Biolab. [³H (G)]taurocholic acid (1 Ci/mmol) was purchased from New England Nuclear (NEN). pDsRed2-ER (6982-1) plasmid was purchased from Clontech. All other chemicals and reagents were purchased from Ambion, Amersham Biosciences, Biorad, Fermentas, Integrated DNA Technologies, Invitrogen, Jackson Immunology Research Lab, J. T., Baker Inc., New England Biolab, Perkin-Elmer, Qiagen, Roche, Sigma-Aldrich, or Stratagene. C57B1/6 mice were purchased from Charles River Laboratories (Kingston, N.Y.) and were kept under a 12 hours light cycle at room temperature. All experimental protocols involving animals were approved by the local Animal Care and Use Committee, according to criteria outlined in the “Guide for the Care and Use of Laboratory Animals” prepared by the National Academy of Sciences, as published by the National Institutes of Health (NIH publication 86-23, revised 1985).

Generation of Ostα^{-/-} Mice. A targeting vector was designed to replace exons 3-9 of Ostα (cDNA nucleotides 490-1545, or a deletion of ˜4.3 kb) with a neomycin (Neo)-containing cassette (Caliper Life Sciences/Xenogen, Cranbury, N.J.). The 5′ homologous arm (2.4 kb) was generated by PCR using BAC clone RP23-123E9 and proofreading LA Taq DNA polymerase (Takara) and cloned into the pCRXL-TOPO vector. The 3′ homologous arm (7.5 kb) was generated by RED cloning/gap repair, and cloned into the FtNwCD vector. Both plasmids were confirmed by restriction digestion and end sequencing. The final FtNwCD vector contained Neo and DTA (diphtheria toxin fragment A) expression cassettes for positive and negative selection in ES cells, respectively, and the sequence was confirmed by both restriction digestion and end sequencing analysis. The vector was linearized with NotI before electroporation into C57BL/6 ES cells (Caliper Life Sciences/Xenogen, Cranbury, N.J.). G418-resistant ES clones were analyzed by Southern blot analysis, the selected clones injected into FVB blastocysts, and the blastocysts implanted into uteri of pseudo-pregnant females for generation of chimeras. Male chimeric mice were bred, and the resulting progeny were genotyped until heterozygous germline transmission was achieved. Heterozygous animals (Ostα^{+/-}) were bred to generate Ostα^{-/-} mice, and all animals were maintained on a standard laboratory diet (LabDiet, PMI Nutrition International, Saint Louis, Mo.) at the University of Rochester School of Medicine and Dentistry Vivarium. Genotyping was performed by PCR analysis of DNA isolated from tail biopsies. Genomic DNA was isolated from 0.5 cm of tail using DirectPCR-Tail lysis reagent (Viagen Biotech, Inc., Los Angeles, Calif.). One μl of crude lysate was used in a one-step genotyping PCR using primers for amplification of a ˜600 bp segment of the neomycin cassette (Fwd 5′-CTGTGCTCGACGTTGTCACTG-3′, SEQ ID NO:98 and Rev 5′-GATCCCCTCAGAAGAACTCGT-3′, SEQ ID NO:99), and an ˜800 bp region of Ostα genomic DNA containing exons 4 and 5 (Fwd 5′-ATTTGTGGTGTCAGTTCTCCTGTCT-3′, SEQ ID NO:100 and Rev 5′-TATTATTGGCTTTGCCCTACACAAG-3′, SEQ ID NO:101), which were designed with Primer Express 1.5.

Protein and Total mRNA Isolation from Various Mouse Tissues. Wild type and Ostα^{-/-} mice were anesthetized with pentobarbital sodium, 50 mg/kg, ip. For protein isolation, liver, kidney and ileum were homogenized in buffer A (10 mM Tris-HCl pH=7.4, 10 mM KCl, 1.5 mM MgCl₂, 200 μg/mL EDTA, 0.25 M sucrose, 0.625% (v/v) protease inhibitor cocktail (Sigma-Aldrich P8340), and 2 mM PMSF), and then centrifuged at 800×g for 20 mM at 4° C. The supernatant is the protein lysate for each tissue. For RNA isolation, liver, kidney, duodenum, jejunum and ileum were collected. Tissue samples not immediately processed for RNA isolation were stored in RNAlater (Ambion). Total mRNA was isolated using Rneasy Midi Kit (Qiagen).

Mouse Ileum Membrane Fraction Isolation. Ileum from C57BL/6 mice were homogenized in buffer A, and then centrifuged at 800×g for 20 mM at 4° C. The supernatant was centrifuged at 32,000×g for 20 mM at 4° C. Mouse ileum membrane fraction was obtained by re-suspending the pellet of the second spin in buffer B (10 mM Tris-HCl pH=7.4, 200 μg/mL EDTA, 0.125 M sucrose, 0.625% (v/v) protease inhibitor cocktail, and 2 mM PMSF).

Co-Immunoprecipitation and Immunoblotting.

Immunoprecipitations (IP) in mouse ileum membrane fractions and in transfected HEK293 cells were performed using the protein G immunoprecipitation kit (IP-50) from Sigma-Aldrich. After pre-clearing with protein G agarose, mouse ileum membrane fraction or proteins extracted from transfected HEK293 cells (˜300 μg) were solubilized in 250 μl of RIPA buffer (50 mM Tris-HCl pH 7.4, 1% NP-40, 0.25% Na-deoxycholate, 150 mM NaCl, 1 mM EDTA, 1 mM PMSF, and 0.625% (v/v) protease inhibitor cocktail). Proteins were incubated with 2.5 μg of anti-Ostα (mA315), anti-Ostβ (mB91), anti-Mrp1, anti-FLAG, or 2 μL anti-c-myc antibodies overnight at 4° C. Immunocomplexes were isolated by incubation with 50 μL of protein G-agarose for 2 hours at room temperature. The agarose beads were washed four times with PBS, and the product of the immunoprecipitation was eluted by adding 90 μL of 1×SDS sample buffer (40 mM Tris-HCl, 4% glycerol, 2% SDS) with β-mercaptoethanol, incubating for 30 mM at 37° C. The protein in the elution was separated by 4-20% SDS-PAGE gel (Bio-Rad). The separated polypeptides were electrotransferred onto a PVDF membrane (Bio-Rad) for 90 minutes at 95 volts using a wet transfer apparatus (Bio-Rad). The membranes were blocked overnight in 5% milk TBST at 4° C. Antibodies were used at the following dilutions: anti-Ostα (mA315) and anti-Ostβ (mB91) at 1:1000, and the HRP-conjugated anti-rabbit secondary antibody at 1:3000 (Amersham Biosciences). Antibody binding was then detected using an enhanced chemiluminescence technique (Perkin-Elmer).

Immunofluorescence Staining. HEK293 cells were grown on lysine-coated 4-well chambered coverslips (Lab-Tek II) and transiently transfected with 0.3 μg each of the following plasmids: Ostα-FLAG or Ostβ-c-myc (negative controls), both Ostα-FLAG and Ostβ-c-myc, or both Ostα-FLAG and c-myc-Ostβ. For surface antigen immunofluorescence, the cells were not permeabilized. Cells were fixed for 15 min in 4% formaldehyde in PBS²⁺ (PBS containing 1 mM MgCl₂ and 0.1 mM CaCl₂), blocked for 30 min with 5% normal goat serum in PBS²⁺, and then incubated at room temperature for 1 hour with 1:50 diluted antibody for c-myc (M 5546) or 1:20 anti-FLAG (F 3165) antibody. Subsequently, the cells were incubated at room temperature for 1 hour with anti-mouse antibody rhodamine—conjugated secondary antibodies (Jackson Immunology Research Lab) at the concentration of 30 μg/mL and then mounted in Prolong® gold antifade reagent with 4′, 6-diamidino-2-phenylindole (DAPI; Invitrogen P36935). Labeled cells were analyzed with an Olympus AH-2 microscope using 20× or 40× air immersion objective lenses. When the cells were permeabilized, a similar procedure was used, except that the blocking and the antibody incubation solutions were supplemented with 0.1% saponin.

Protein Stability Assay. HEK293 cells were transfected with Ostα-FLAG, mOstβ-c-myc or both (0.1 μg each plasmid/3-cm plate). Twenty-four hours after transfection, the cells were changed to medium with or without 100 μg/ml of cycloheximide (Sigma-Aldrich), a protein synthesis inhibitor. Cells were harvested at different time points and processed for immunoblotting using anti-FLAG or anti-c-myc antibodies.

Real-time Quantitative Reverse-Transcriptase PCR Analyses. Gene-specific primers for Ostβ were as previously described (Ballatori et al., “Ostα-Ostβ, a Major Basolateral Bile Acid and Steroid Transporter in Human Intestinal, Renal, and Biliary Epithelia,” Hepatology 42:1270-1279 (2005); and Boyer et al., “Upregulation of a Basolateral Fxr-Dependent Bile Acid Efflux Transporter Ostα-Ostβ in Cholestasis in Humans and Rodents,” Am. J. Physiol. Gastrointest. Liver Physiol. 290:G11240-30 (2006), which are hereby incorporated by reference in their entirety). Relative gene expression was determined on a Corbett Rotor-Gene 3000 real-time cycler. Samples were analyzed using an iScript One-step reverse-transcriptase PCR kit with SYBR Green (Bio-Rad).

Production of Fluorescent Fusion Proteins. The sequences encoding YFP (1-154) and YFP (155-238) were kindly provided by Dr. Tom K. Kerppola, University of Michigan Medical School, Ann Arbor, Mich. (Hu et al., “Visualization of Interactions Among Bzip and Rel Family Proteins in Living Cells Using Bimolecular Fluorescence Complementation,” Mol. Cell. 9:789-798 (2003), which is hereby incorporated by reference in its entirety) and cloned into pCMV-HA (Clonetech) and pCMV-FLAG2 (Sigma-Aldrich). The sequences encoding YFP (1-158) and YFP (159-238) were generously provided by Dr. Catherine H. Berlot, Weis Center for Research, Geisinger Clinic, Danville, Pa. (Hynes et al., “Visualization of G Protein Betagamma Dimers Using Bimolecular Fluorescence Complementation Demonstrates Roles for Both Beta and Gamma in Subcellular Targeting,”J. Biol. Chem. 279:30279-86 (2004), which is hereby incorporated by reference in its entirety) and cloned into pDNAI/Amp. To construct mammalian expression vectors for bimolecular fluorescence complementation (BiFC) analysis, the mouse Ostα sequence was fused to the N-terminal end of YFP (1-154), YFP (155-238) or the C-terminal end of YFP (1-158) to produce Ostα-YN, Ostα-YC, and YN-Ostα. Mouse Ostβ was fused to the N-terminal end of YFP (155-238) or C-terminal end of YFP (159-238) to produce Ostβ-YC or YC-Ostβ. The constructs were verified by DNA sequencing.

Transient Expression and Assay for Bile Acid Transport Activity. HEK293 cells were maintained under conditions recommended by the American Type Culture Collection. Cells grown in six-well plates to 70-80% confluence were transfected with 0.1-1 μg of the plasmids expressing the proteins indicated in each experiment using Fugene 6 (Roche). After 24 hour of culture, the cells were incubated at 37° C. for 30 mM in Krebs Henseleit (KH) buffer (118 mM NaCl, 4.7 mM KCl, 1.2 mM KH₂PO₄, 25 mM NaHCO₃, 0.6 mM MgSO₄, 1.25 mM CaCl₂, 5 mM Hepes/Tris), containing 25 μM [³H]taurocholic acid. After incubation, the culture medium was removed, and each cell monolayer was washed three times with ice-cold KH buffer containing 0.2% (wt/vol) bovine serum albumin and 1 mM taurocholate, and then once more with ice-cold KH buffer alone. The cell monolayer was dissolved in 0.1M NaOH and aliquots were taken to determine cell-associated protein and radioactivity.

Imaging of Fluorescent Fusion Proteins. HEK293 cells grown in 35 mm glass bottom dishes (MatTek P-35G-0-14-C) to 50% confluence were transfected with 0.25-0.5 μg of various plasmids using Fugene 6. Transfected cells were incubated at 37° C. for 24 hour and then switched to 30° C. for 2 hours to promote fluorophore maturation. The cells were observed on a Leica TCS SP Spectral confocal microscope, equipped with an inverted DMIRBE microscope. YFP fluorescence emission was measured at 555/15 nn. DsRed2 fluorescence was measured at 610/30 nm.

Statistical Analyses. Data are given as means±SEM. Mean values were considered to be significantly different, when P<0.05 by use of an one-way ANOVA followed by Bonferroni's multiple comparison test.

### Example 1

### Mouse Ileal Ostα and Ostβ Proteins Appear as Multiple Bands on Western Blots

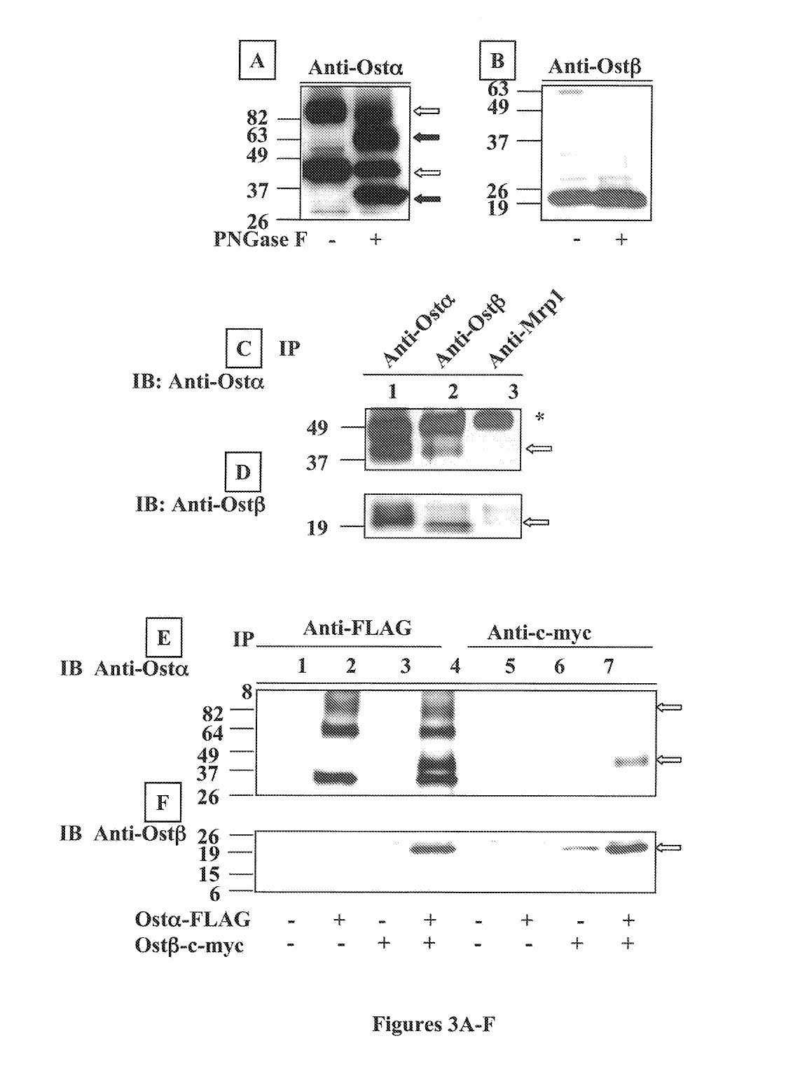

In the mouse, Ostα and Ostβ proteins are most abundant in kidney and small intestine, and are especially high in the ileum (Dawson et al., “The Heteromeric Organic Solute Transporter α-β, Ostα-Ostβ, is an Ileal Basolateral Bile Acid Transporter,” J. Biol. Chem. 280:6960-6968 (2005), which is hereby incorporated by reference in its entirety). To determine the relative molecular sizes of Ostα and Ostβ in the ileum and to test for possible protein complexes, affinity-purified polyclonal anti-peptide antibodies for mouse Ostα (mA315) and Ostβ (mB91) were used in Western blot analysis of mouse ileum membrane proteins. Ostα was detected as a predominant protein band of ˜40 kDa, as well as bands of ˜50 and 80 kDa, although the ˜50 kDa band was faint and not always detectable (FIG. 3A). Comparable results were observed when the gel was run under non-reducing conditions. The predicted mouse Ostα molecular weight is 37.8 kDa, and thus the 40 kDa band is likely the monomer form of Ostα (Dawson et al., “The Heteromeric Organic Solute Transporter α-β, Ostα-Ostβ, is an Ileal Basolateral Bile Acid Transporter,” J. Biol. Chem. 280:6960-6968 (2005), which is hereby incorporated by reference in its entirety), whereas the 80 kDa band may represent Ostα that is either post-translationally modified or complexed with another protein, including perhaps Ostαitself (i.e., the Ostα homodimer).

To test whether the higher molecular band of Ostα is a glycosylated form, mouse ileum membrane proteins were treated with PNGase F. As expected (Dawson et al., “The Heteromeric Organic Solute Transporter α-β, Ostα-Ostβ, is an Ileal Basolateral Bile Acid Transporter,” J. Biol. Chem. 280:6960-6968 (2005), which is hereby incorporated by reference in its entirety), PNGase F treatment led to a decrease in the 40 kDa Ostα band and the appearance of a 36 kDa band (FIG. 3A, lane 2). Interestingly, the size of 80 kDa band also decreased after treatment with PNGase F, and a new band was formed at about ˜60 kDa (FIG. 3A, lane 2). The 80 kDa band was not sensitive to Endo H treatment.

For Ostβ, bands were detected at around 19 kDa and 17 kDa, although these bands often overlapped (FIG. 3B). The predicted mouse Ostβ molecular size is about 14.7 kDa. The Ostβ antibody was not able to detect the ˜50 and 80 kDa bands stained by anti-Ostα, indicating that these higher molecular weight bands are not heteromeric complexes of Ostα and Ostβ. The 17 and 19 kDa bands of Ostβ were minimally affected by PNGase F (FIG. 3B).

### Example 2

### Co-Immunoprecipitation of Ostα and Ostβ

To examine whether Ostα and Ostβ associate directly, immunoprecipitation and immunoblot analyses were carried out using mouse ileum membrane proteins. The mA315 (anti-Ostα) antibody was used to immunoprecipitate the Ostα subunit and the precipitated proteins were probed with mA315 and mB91 antibodies. The experiment was also performed in the opposite direction using the anti-Ostβ (mB91) to immunoprecipitate Ostβ. Mrp1, a transport protein that is also located in the basolateral membrane, was chosen as a negative control.

Immunoprecipitation of Ostβ from mouse ileum resulted in the co-immunoprecipitation of a 40 kDa protein that was detected by the anti-Ostα antibody (FIG. 3C, lane 2). Likewise a 19 kDa Ostβ-reactive band was co-immunoprecipitated by the Ostα antibody (FIG. 3D, lane 1), indicating that the two proteins are associated with each other. Anti-Mrp1 antibody did not pull down any band corresponding to Ostα (FIG. 3C, lane 3, open arrow), and only a faint signal was detected with the anti-Ostβ antibody (FIG. 3D, lane 3, open arrow). Because the antibodies used to immunoprecipitate and immunoblot were both rabbit polyclonal antibodies, some extra bands were created that obscured the ˜50 and ˜80 kDa bands. Thus, the higher molecular weight Ostα bands which were detected by mA315 (FIG. 3A) were not distinguishable in this experiment.

To confirm these findings and examine whether the higher molecular weight forms of Ostα also interact with Ostβ, co-immunoprecipitation studies were also performed in transfected HEK293 cells. Mouse Ostα was labeled with the FLAG epitope and mouse Ostβ was tagged with the c-myc epitope on their carboxyl termini. The epitope tags allowed the use of different antibodies for immunoprecipitation and immunoblotting. When Ostα-FLAG was expressed by itself at relatively high levels (1 μg DNA/3-cm plate), bands at ˜36, 60 and 80 kDa were formed (FIG. 3E, lane 2); however, when co-expressed with Ostβ-c-myc a new ˜40 kDa mA315 antibody-reactive band was formed (FIG. 3E, lane 4), consistent with glycosylation of Ostα (Dawson et al., “The Heteromeric Organic Solute Transporter α-β, Ostα-Ostβ, is an Ileal Basolateral Bile Acid Transporter,” J. Biol. Chem. 280:6960-6968 (2005), which is hereby incorporated by reference in its entirety). When the same samples were blotted with anti-Ostβ antibody, a prominent ˜19 kDa band was seen only in cells co-expressing Ostα-FLAG and Ostβ-c-myc (FIG. 3F, lane 4), indicating that precipitation of Ostα resulted in the co-immunoprecipitation of Ostβ. When the anti-c-myc antibody was used to precipitate Ostβ-c-myc, bands were detected with the mA315 antibody at ˜40 kDa and ˜80 kDa, whereas the 36 kDa and the ˜60 kDa were not detected (FIG. 3E lane 8). These results support the findings obtained in mouse ileum, and suggest that the ˜80 kDa form of Ostα also interacts with Ostβ.

### Example 3

### Visualization of Ostα-Ostβ Heterodimers or Heteromultimers in Living HEK293 Cells

To confirm the immunoprecipitation results, and to localize the putative Ostα-Ostβ heterodimer or heteromultimers in living cells, BiFC analysis was performed. Although HEK293 cells express human Ostα and Ostβ mRNA, the levels are quite low (approximately 36 and 49 copies/ng total RNA, respectively), and did not interfere in the analysis of the heterologously expressed mouse genes. An amino-terminal YFP fragment (residues 1-154) was fused to the carboxyl-terminus of mouse Ostα to produce Ostα-YN, and a carboxyl-terminal YFP fragment (residues 155-238) was fused to the carboxyl-terminus of mouse Ostβ to produce Ostβ-YC. The pDsRed2-ER plasmid (Clonetech) was co-transfected as a marker of the endoplasmic reticulum, and the bJun-YN and bFos-YC plasmids were used as a BiFC positive control. As reported previously (Hu et al., “Visualization of Interactions Among Bzip and Rel Family Proteins in Living Cells Using Bimolecular Fluorescence Complementation,” Mol. Cell. 9:789-798 (2003), which is hereby incorporated by reference in its entirety), bJun-YN-bFos-YC associated preferentially in nuclei (FIGS. 4A-C). When Ostα-YN was co-expressed with untagged Ostβ, or when Ostβ-YC was co-expressed with untagged Ostα no fluorescence signal was detected, as expected; however, when Ostα-YN and Ostβ-YC were co-expressed, a fluorescence signal was detected at both the plasma membrane and intracellularly (FIGS. 4E and F). The intracellular Ostα-YN-Ostβ-YC green staining partially co-localized with the red endoplasmic reticulum staining, suggesting that Ostα-YN and Ostβ-YC interact in the endoplasmic reticulum and that the resulting complex(es) then proceed to the cell membrane.

To confirm that the putative Ostα-YN-Ostβ-YC complex is functionally active, [³H]taurocholate transport activity was measured in the transfected cells. As expected (Seward et al., “Functional Complementation Between a Novel Mammalian Polygenic Transport Complex and an Evolutionarily Ancient Organic Solute Transporter, Ostα-Ostβ,”J. Biol. Chem. 278:27473-27482 (2003) and Dawson et al., “The Heteromeric Organic Solute Transporter α-β, Ostα-Ostβ, is an Ileal Basolateral Bile Acid Transporter,” J. Biol. Chem. 280:6960-6968 (2005), which are hereby incorporated by reference in their entirety), co-expression of untagged Ostα-Ostβ generated taurocholate transport activity (FIG. 4G). Note that roughly comparable taurocholate transport activity was observed when Ostα-YN and Ostβ-YC were co-expressed (FIG. 4G), indicating that the carboxyl-terminal YFP fragment constructs are able to reach the plasma membrane and generate a functional transporter.

### Example 4

### Ostα Forms Homodimers or Homomultimers, and Ostβ is Required for Delivery of the Complex to the Plasma Membrane

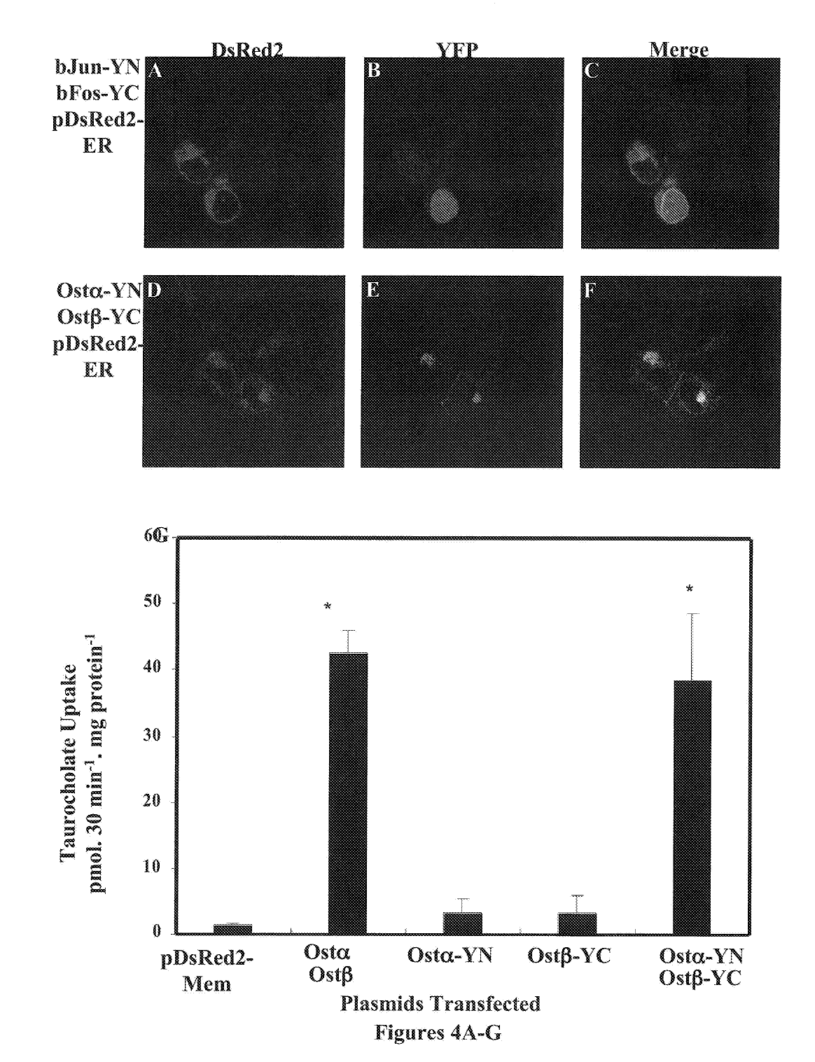

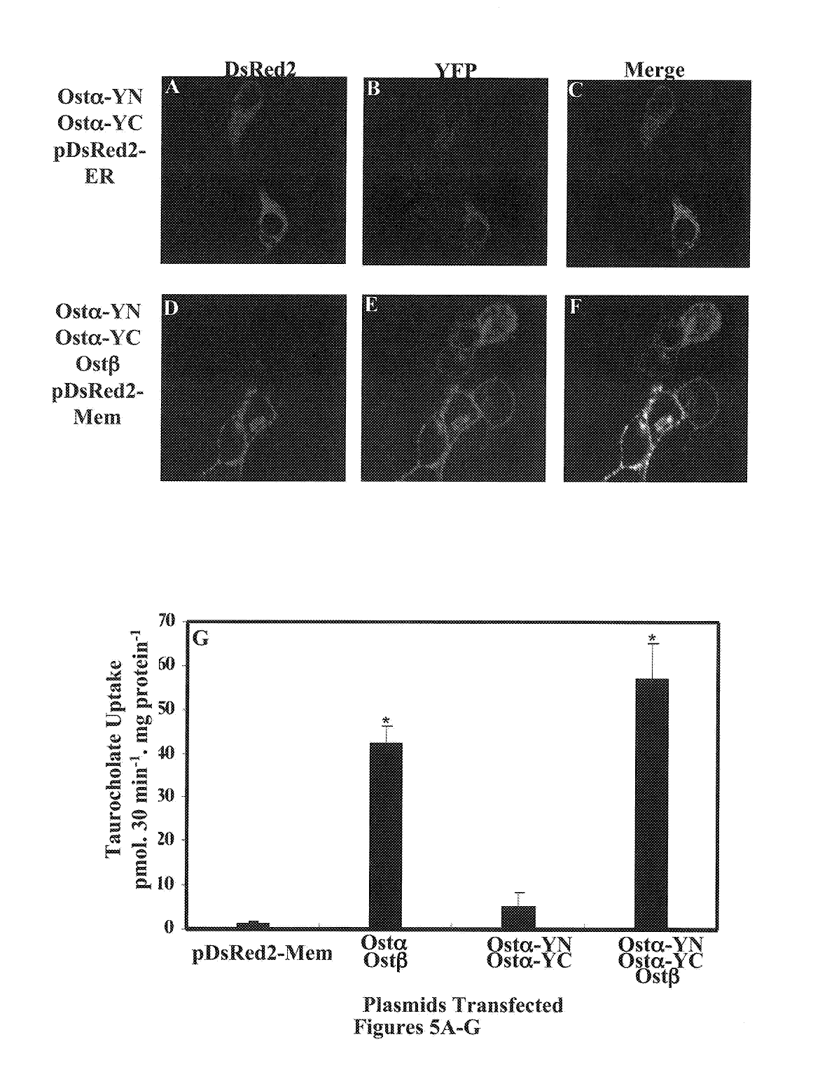

To directly test the possibility that Ostα is forming a homodimer and/or homomultimers, BiFC analysis was performed using two different Ostαconstructs; one tagged with the YFP carboxyl-terminal fragment (Ostα-YC), and the other with the amino-terminal fragment (Ostα-YN). When these two constructs were co-expressed in HEK293 cells, the total amount of YFP signal was relatively low, but was detected in a number of cells, indicating that Ostα-YN interacts with Ostα-YC (FIGS. 5B and C). In contrast with the plasma membrane localization of the heteromeric Ostα-YN-Ostβ-YC complex (FIGS. 4E and F), the YFP signal of Ostα-YN-Ostα-YC largely overlapped that of the endoplasmic reticulum marker and there was no significant plasma membrane staining (FIG. 5C).

On the other hand, when Ostβ was transfected along with Ostα-YN and Ostα-YC, a strong YFP signal was now observed at the plasma membrane (FIGS. 5D-F). In this experiment, a marker of the plasma membrane was used, pDsRed2-Mem, which encodes for a fluorescent fusion protein that is targeted to plasma membrane by the amino-terminal 20 residues of neuromodulin (Zuber et al., “A Membrane-Targeting Signal in the Amino Terminus of the Neuronal Protein Gap-43,” Nature 341:345-348 (1989), which is hereby incorporated by reference in its entirety) (FIG. 5D). As illustrated in FIGS. 5E and 5F, the fluorescence signal overlapped with that of the pDsRed2-Mem signal, confirming that the complex localizes to the plasma membrane.

Measurements of bile acid transport activity revealed that cells transfected with Ostα-YN and Ostα-YC showed no significant increase in taurocholate transport activity; however, when Ostβ was co-transfected along with Ostα-YN and Ostα-YC, taurocholate transport activity was now comparable to that generated by untagged Ostα and Ostβ (FIG. 5G).

### Example 5

### Membrane Topology of Ostα and Ostβ

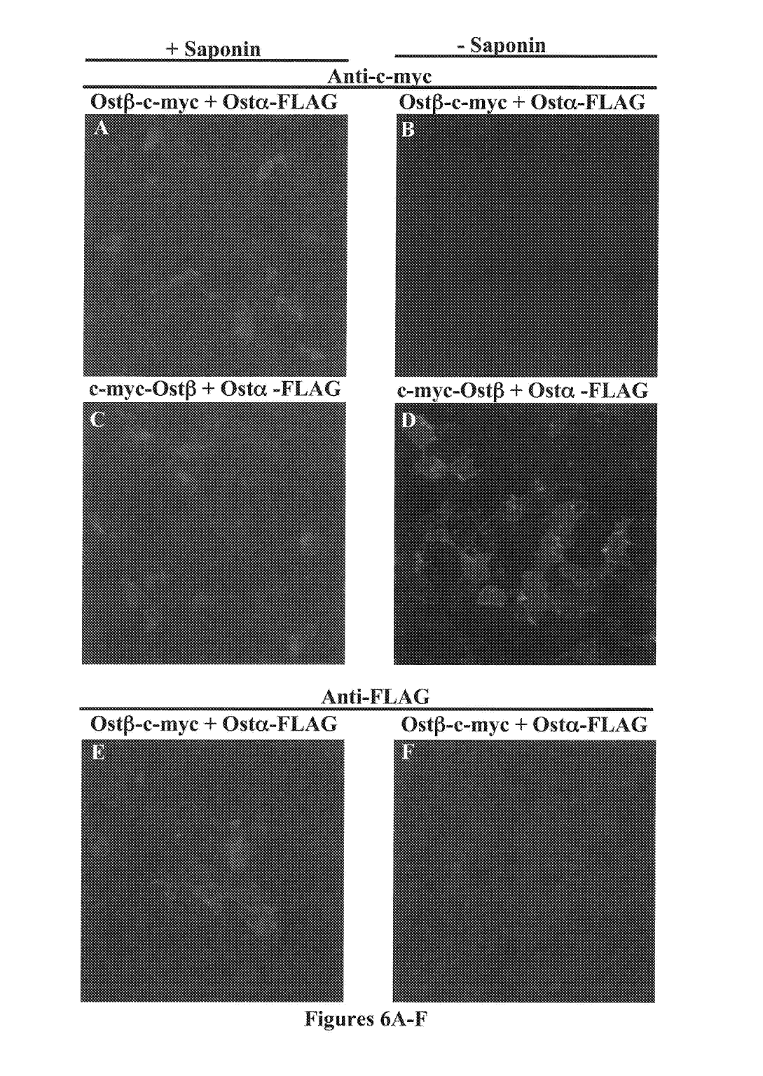

Based on the evolutionarily conserved 7-TM domain predicted architecture of Ostα, this protein is thought to have an extracellular amino terminus and an intracellular carboxyl terminus (Seward et al., “Functional Complementation Between a Novel Mammalian Polygenic Transport Complex and an Evolutionarily Ancient Organic Solute Transporter, Ostα-Ostβ,”J. Biol. Chem. 278:27473-27482 (2003), which is hereby incorporated by reference in its entirety). On the other hand, Ostβ is thought to have a single TM domain (Seward et al., “Functional Complementation Between a Novel Mammalian Polygenic Transport Complex and an Evolutionarily Ancient Organic Solute Transporter, Ostα-Ostβ,” J. Biol. Chem. 278:27473-27482 (2003), which is hereby incorporated by reference in its entirety), but its membrane topology is more difficult to predict. To gain insight into the membrane topology of Ostα and Ostβ, immunofluorescence studies were carried out in HEK293 cells transfected with carboxyl terminus tagged Ostα (Ostα —FLAG) along with Ostβ tagged at either its carboxyl or amino termini with the c-myc epitope (Ostβ-c-myc or c-myc-Ostβ, respectively). Both of these combinations (i.e., Ostα-FLAG with either Ostβ-c-myc or c-myc-Ostβ) generated taurocholate transport activity in transfected cells. When cells were permeabilized with 0.1% saponin, both of the combinations were labeled by the anti-c-myc antibody (FIGS. 6A and C); however, when non-permeabilized cells were incubated with anti-c-myc antibody, only cells transfected with the amino terminus-tagged Ostβ construct were labeled (compare FIGS. 6B and D), supporting the conclusion that the carboxyl terminus of Ostβ is intracellular. Likewise, when cells were incubated with anti-FLAG antibody, only cells permeabilized with saponin showed staining (FIGS. 6E and F), indicating that the carboxyl terminus of Ostα is also intracellular.

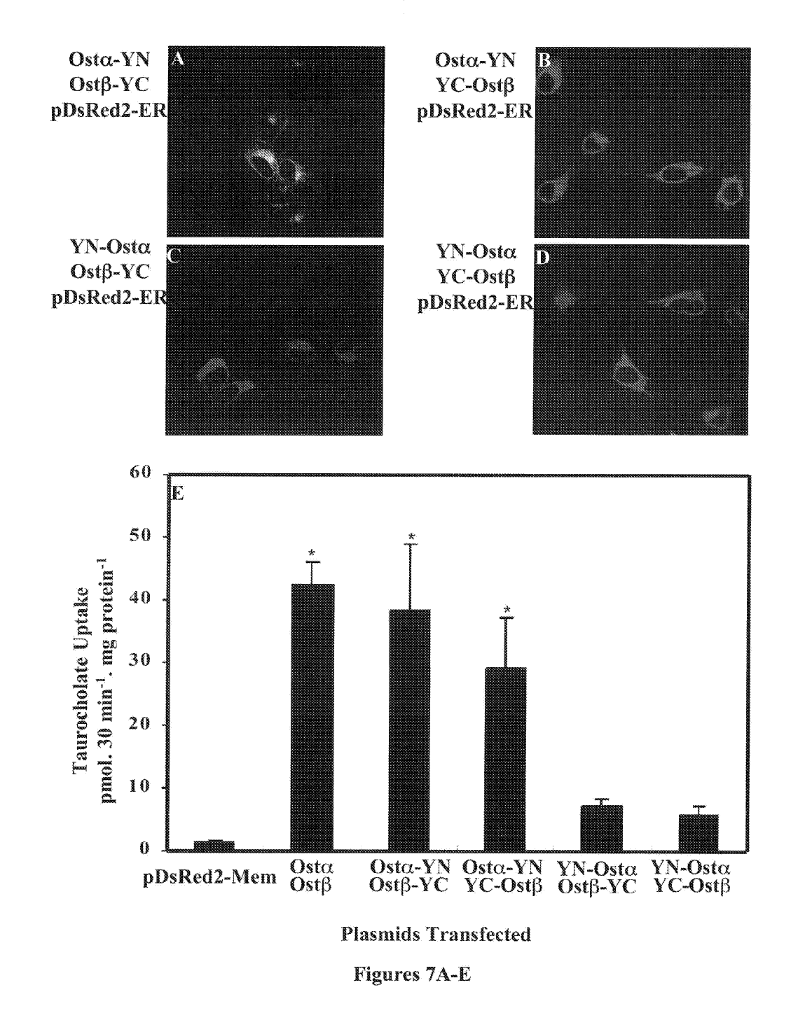

To confirm this membrane topology, BiFC analysis was performed with various combinations of constructs, including YN-Ostα and YC-Ostβ, in which the two YFP fragments were placed on the amino terminal portions of Ostα and Ostβ. HEK293 cells were transfected with either, a) Ostα-YN and Ostβ-YC, b) Ostα-YN and YC-Ostβ, c) YN-Ostα and Ostβ-YC, or d) YN-Ostα and YC-Ostβ, along with pDsRed2-ER as a control for transfection and as a marker of the endoplasmic reticulum (FIGS. 7A-D). FIGS. 7 A-D show the results of the merged YFP and pDsRed2 fluorescence signal, and FIG. 7E provides measurements of their functional activity (taurocholate transport).

Of these four combinations of BiFC constructs, only the Ostα-YN and Ostβ-YC pair was able to elicit a YFP signal (FIG. 7A), indicating that the carboxyl termini of both proteins are on the same side of the membrane, and most likely the cytoplasmic side. These constructs also elicited taurocholate transport activity (FIG. 7E). No fluorescence signal was detected when one YFP fragments was placed on the amino-terminal portion of Ostα and one on the carboxyl-terminal portions of Ostβ, and vice versa (FIG. 7). It was noted, however, that the addition of amino terminal epitope tags to Ostα invariably abolished taurocholate transport activity (FIG. 7E), indicating that such modifications to Ostα generate products are either unstable, cannot be targeted properly to the plasma membrane, or that lack transport activity. Of significance, although the Ostα-YN and YC-Ostβ pair failed to generate a YFP signal, it generated taurocholate transport activity (FIG. 7E), indicating a functional interaction. These observations provide additional direct support for the conclusion that the carboxyl termini of both Ost proteins are on the same side of the plasma membrane, and are most likely intracellular.

### Example 6

### Heterodimerization of Ostα and Ostβ Increases Stability of the Proteins

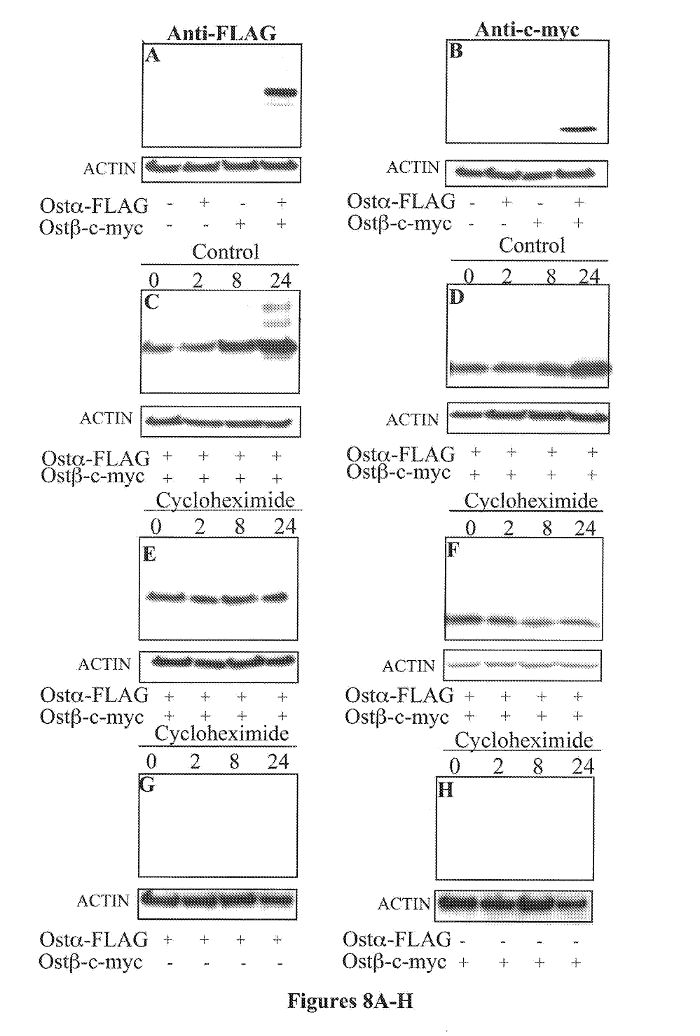

To examine whether the interaction between Ostα and Ostβ influences the turnover rate of these proteins, HEK293 cells were transfected with either Ostα-FLAG, Ostβ-c-myc, or both, and protein abundance was assessed at different time intervals by Western blotting (FIGS. 8A-H). For these studies, a lower level of DNA was used for the transfection, namely 0.1 μg DNA/3-cm dish, versus 1 μg DNA/3-cm dish for the studies illustrated in FIGS. 3E and F. When Ostα or Ostβ were expressed individually, the proteins were not detectable (FIG. 8A lane 2, and 8B lane 3), but were easily detected when Ostα and Ostβ were co-expressed (FIGS. 8A and 8B, lane 4). To assess relative protein stability, the levels of transiently expressed proteins were compared at different time points in the presence and absence of cycloheximide to inhibit protein synthesis. In Ostα-Ostβ co-expressing cells, the Ostα and Ostβ proteins continued to accumulate with time in culture (FIGS. 8C and 8D). In the presence of cycloheximide, protein levels decreased only slightly during the 24 hour period (FIGS. 8E and 8F) but they were not detectable when expressed individually (FIGS. 8G and 8H), suggesting that the proteins are relatively stable when co-expressed.

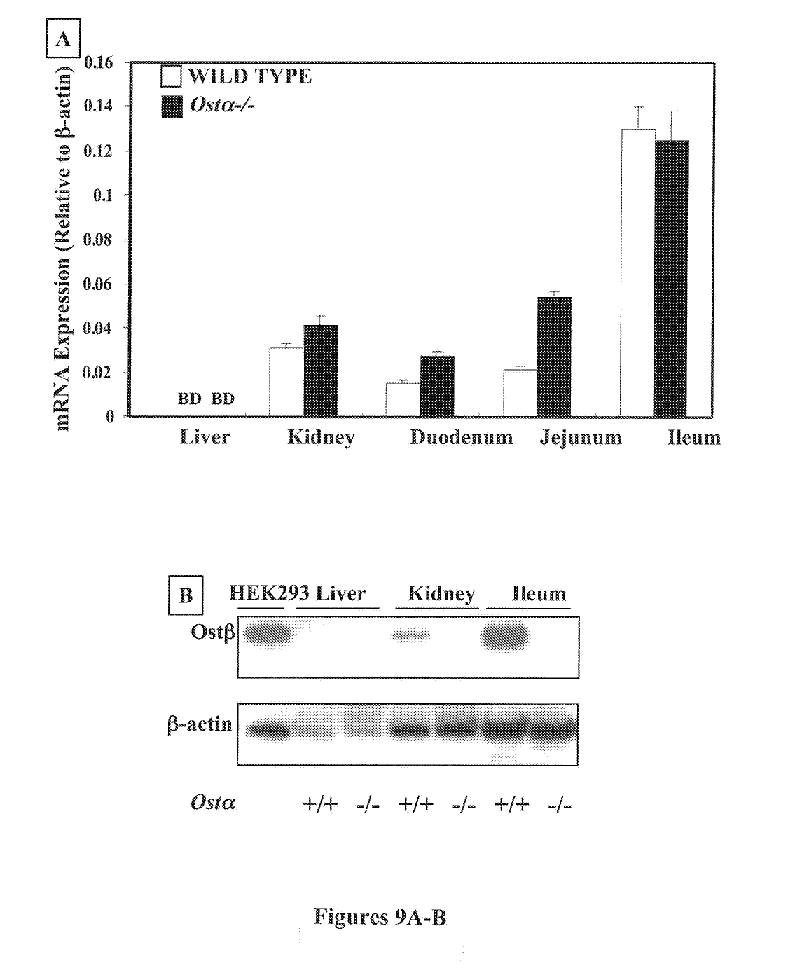

If heterodimerization is required for protein stability, one would predict that the interacting partner should be present at lower levels in cells that are deficient in either Ostα or Ostβ. To test this possibility, studies took advantage of the Ostα-deficient mouse model. Ostα-null mice are viable and fertile, but exhibit growth retardation. Interestingly, Ostβ mRNA levels were maintained in tissues of Ostα^{-/-} mice (FIG. 9A); however, Ostβ protein was not detected in any of the tissues examined (FIG. 9B). Taken together with the findings in HEK293 cells (FIGS. 8A-H), these data suggest that in the absence of their heterodimerization partner, Ostα and Ostβ proteins are rapidly degraded.

### Discussion of Examples 1-6

The present observations provide important insights into Ostα and Ostβ membrane topology, trafficking, and protein stability. Ostα and Ostβ are known to be expressed in parallel in most tissues, such that cells that express high levels of Ostα also express relatively high levels of Ostβ, and vice versa (Seward et al., “Functional Complementation Between a Novel Mammalian Polygenic Transport Complex and an Evolutionarily Ancient Organic Solute Transporter, Ostα-Ostβ,” J. Biol. Chem. 278:27473-27482 (2003); Dawson et al., “The Heteromeric Organic Solute Transporter α-β, Ostα-Ostβ, is an Ileal Basolateral Bile Acid Transporter,” J. Biol. Chem. 280:6960-6968 (2005); Ballatori et al., “Ostα-Ostβ, a Major Basolateral Bile Acid and Steroid Transporter in Human Intestinal, Renal, and Biliary Epithelia,” Hepatology 42:1270-1279 (2005); and Ballatori, “Biology of a Novel Organic Solute and Steroid Transporter, Ostα-Ostβ,” Exp. Biol. Med. 230:689-698 (2005), which are hereby incorporated by reference in their entirety). Ostα and Ostβ are both localized to the basolateral plasma membrane of polarized intestinal, renal and biliary epithelial cells, and in vitro transfection studies show that co-expression of Ostα and Ostβ is required for delivery of the individual proteins to the plasma membrane (Dawson et al., “The Heteromeric Organic Solute Transporter α-β, Ostα-Ostβ, is an Ileal Basolateral Bile Acid Transporter,” J. Biol. Chem. 280:6960-6968 (2005); and Ballatori et al., “Ostα-Ostβ, a Major Basolateral Bile Acid and Steroid Transporter in Human Intestinal, Renal, and Biliary Epithelia,” Hepatology 42:1270-1279 (2005), which are hereby incorporated by reference in their entirety). Previous work has demonstrated that co-expression is also required to convert the Ostα subunit to a mature N-glycosylated, Endo H-resistant form, indicating that co-expression facilitates the movement of Ostα through the Golgi apparatus (Dawson et al., “The Heteromeric Organic Solute Transporter α-β, Ostα-Ostβ, is an Ileal Basolateral Bile Acid Transporter,” J. Biol. Chem. 280:6960-6968 (2005), which is hereby incorporated by reference in its entirety). The present results extend these findings by showing that an Ostα-Ostβ heteromeric complex is apparently formed in the ER, is modified as it transits through the Golgi apparatus, and is then targeted to the plasma membrane. In contrast, when Ostα and Ostβ are expressed individually, the proteins appear to be targeted for degradation.

Evidence for a direct interaction between Ostα and Ostβ was obtained from co-immunoprecipitation and BiFC analyses, two powerful approaches for examining protein-protein interactions (Phizicky et al., “Protein-Protein Interactions: Methods for Detection and Analysis,” Microbiol. Rev. 59:94-123 (1995); Kerppla, “Visualization of Molecular Interactions by Fluorescence Complementation,” Nat. Rev. Mol. Cell. Biol. 7:449-56 (2006); and Kerppla, “Complementary Methods for Studies of Protein Interactions in Living Cells,” Nat. Methods 3:969-71 (2006), which are hereby incorporated by reference in their entirety). The use of primary antibodies to immunoprecipitate the native proteins in mouse ileum provides direct evidence for a physiologically relevant interaction, and the results of the BiFC analysis of transfected cells provides insight into the subcellular localization of the complex in intact cells.

A recent report by Sun and coworkers (Sun et al., “Protein-Protein Interaction and Membrane Localization of Human Organic Solute Transporter (Host),” Am. J. Physiol. Gastrointest. Liver Physiol. 292(6):G1586 (2007), which is hereby incorporated by reference in its entirety) proposes that the amino-terminal 50 amino acid residues of Ostα may be important for the interaction between Ostα and Ostβ. These investigators demonstrated that truncation of this stretch of amino acids in human Ostα led to the intracellular accumulation of Ostα and Ostβ and to only background levels of taurocholate transport activity. However, this truncation may have also affected Ostα biogenesis and folding, but these possibilities were not examined. The results of Examples 1-6 have revealed that although epitope tags are well tolerated on the carboxyl terminus of Ostα (e.g., see FIGS. 4-7), tags placed on the amino terminus abolished taurocholate transport activity (FIG. 7E), indicating that such modifications result in proteins that are either unstable, cannot be targeted properly to the plasma membrane, or lack transport activity.

Co-expression of Ostα and Ostβ was not only required for membrane targeting, but it also appeared to protect the proteins from degradation. Studies with other multimeric protein complexes have established that folding and assembly of such complexes are tightly controlled processes that ensure the expression of an appropriate number of fully assembled complexes on the cell surface. The assembly of multimeric protein complexes is often directly coupled to the trafficking of their individual components, preventing incompletely assembled complexes from reaching the cell surface (Margeta-Mitrovic et al., “A Trafficking Checkpoint Controls Gaba (B) Receptor Heterodimerization,” Neuron 27:97-106 (2000), which is hereby incorporated by reference in its entirety). In the preceding Examples, when Ostα-YN and Ostα-YC were co-expressed in the absence of Ostβ, the resulting homodimer was retained within the cell, and mainly in the ER. However, in the presence of Ostβ, the fluorescence was stronger and was mainly distributed at the plasma membrane (FIG. 5). The altered subcellular YFP localization after transfection with Ostβ indicates that the trafficking of Ostα is regulated by Ostβ. Moreover, the smaller YFP signal from Ostα-YN-Ostα-YC indicates that this homodimer is not as stable in the absence of Ostβ, and it suggests that Ostβ somehow facilitates the movement of the Ostα homodimer through the ER checkpoint. As demonstrated by Dawson et al., (“The Heteromeric Organic Solute Transporter α-β, Ostα-Ostβ, is an Ileal Basolateral Bile Acid Transporter,” J. Biol. Chem. 280:6960-6968 (2005), which is hereby incorporated by reference in its entirety), co-expression facilitates the glycosylation of Ostα. Another possibility is suggested by studies showing that individual subunits of some ion channels or receptor complexes contain discrete ER retention signal(s) that are hidden or overcome by forward trafficking signals in the correctly assembled protein complexes (Letourneur et al., “A Novel Di-Leucine Motif and a Tyrosine-Based Motif Independently Mediate Lysosomal Targeting and Endocytosis of CD3 Chains,” Cell 69:1143-1157 (1992); Letourneur et al., “Steric Masking of a Dilysine Endoplasmic Reticulum Retention Motif During Assembly of The Human High Affinity Receptor for Immunoglobulin,” E. J. Cell Biol. 129:971-978 (1995); and Teasdale et al., “Signal-Mediated Sorting of Membrane Proteins Between the Endoplasmic Reticulum and Golgi Apparatus,” Annu. Rev. Cell Dev. Biol. 12:27-54 (1996), which are hereby incorporated by reference in their entirety). The unassembled proteins would be retained in the ER. Interestingly, mouse Ostβ has an Arg-X-Arg (RXR) motif in its carboxyl terminus, whereas Ostα has an RRK sequence at its carboxyl terminus, and both have di-leucine motifs that have been demonstrated to function as endoplasmic reticulum retention/retrieval signals, which prevent the cell surface expression of individual subunits and partially assembled complexes (Margeta-Mitrovic et al., “A Trafficking Checkpoint Controls Gaba (B) Receptor Heterodimerization,” Neuron 27:97-106 (2000); Ellgaard et al., “Setting the Standards: Quality Control in the Secretory Pathway,” Science 286:1882-1888 (1999); Zerangue et al., “A New ER Trafficking Signal Regulates the Subunit Stoichiometry of Plasma Membrane K(Atp) Channels,” Neuron 22:537-548 (1999); and Ma et al., “ER Transport Signals and Trafficking of Potassium Channels and Receptors,” Curr. Opin. Neurobiol. 12:287-92 (2002), which are hereby incorporated by reference in their entirety). Thus, one possibility is that Ostβ functions to sequester this retrieval signal in the correctly assembled complex.

Additional evidence that co-expression of Ostα and Ostβ increases protein stability was provided in Example 4 using transfected HEK293 cells and in Example 6 with Ostα^{-/-} mice. When Ostα and Ostβ were expressed individually using moderate levels of DNA for the transfections (0.1 μg plasmid DNA/3-cm plate) the proteins were not detected, and were only seen when co-expressed. Interestingly, in Ostα^{-/-} mice the Ostβ protein was also not detected, although the Ostβ mRNA levels were comparable to those of wild type mice, indicating that the absence of Ostβ protein in knockout mouse occurs post-transcriptionally, and that Ostα expression is required for Ostβ protein stability.

The preceding Examples also provide convincing evidence that the carboxyl termini of both Ostα and Ostβ are located intracellularly. This overall topology of Ostα-Ostβ is similar to that of the GPCR-RAMP complexes (Morfis et al., “RAMPs: 5 Years On, Where to Now?,” Trends Pharmacol. Sci. 24:596-601 (2003); Hay et al., “GPCR Modulation By RAMPs,” Pharmacol. Ther. 109:173-197 (2006); and Bockaert et al., Molecular Tinkering of G Protein-Coupled Receptors: An Evolutionary Success,” EMBO J. 18:1723-1729 (1999), which are hereby incorporated by reference in their entirety). Ostα-Ostβ and GPCR-RAMP both consist of a seven-helix TM protein (i.e., Ostα and GPCRs) and a single-TM accessory polypeptide (i.e., Ostβ and RAMPs) with cytosolic carboxyl terminus. GPCRs are known to be present as homodimers and heterodimers, and GPCR-RAMP complexes as heterodimers and hetero-oligomers (Angers et al., “Dimerization: An Emerging Concept for G Protein-Coupled Receptor Ontogeny and Function,” Annu. Rev. Pharmacol. Toxicol. 42:409-435 (2002); and Bouvier, M., “Oligomerization of G-Protein-Coupled Transmitter Receptors,” Nat. Rev. Neurosci. 2:274-286 (2001), which are hereby incorporated by reference in their entirety). The preceding Examples indicate that the Ostα-Ostβ complex is also likely to be a hetero-oligomer, consisting of two Ostα and one or more Ostβ proteins, although the actual stoichiometry has yet to be defined.

### Material and Methods for Examples 7-11

Materials. HEK293 cells were obtained from ATCC(CRL-1573) and grown as monolayers at 37° C. in an atmosphere of 5% CO₂. Cells were maintained in medium consisting of DMEM (GIBCO, 10-013-CV) with 10% (v/v) fetal calf serum and antibiotics. [³H(G)]Taurocholic acid (2 Ci/mmol) was purchased from PerkinElmer Life and Analytical Sciences, Inc.; the pDsRed2-ER (6982-1) plasmid was purchased from Clontech; and the pDsRed2-MEM, pBiFCC155, and pBiFCN155 plasmids were generated as described infra.

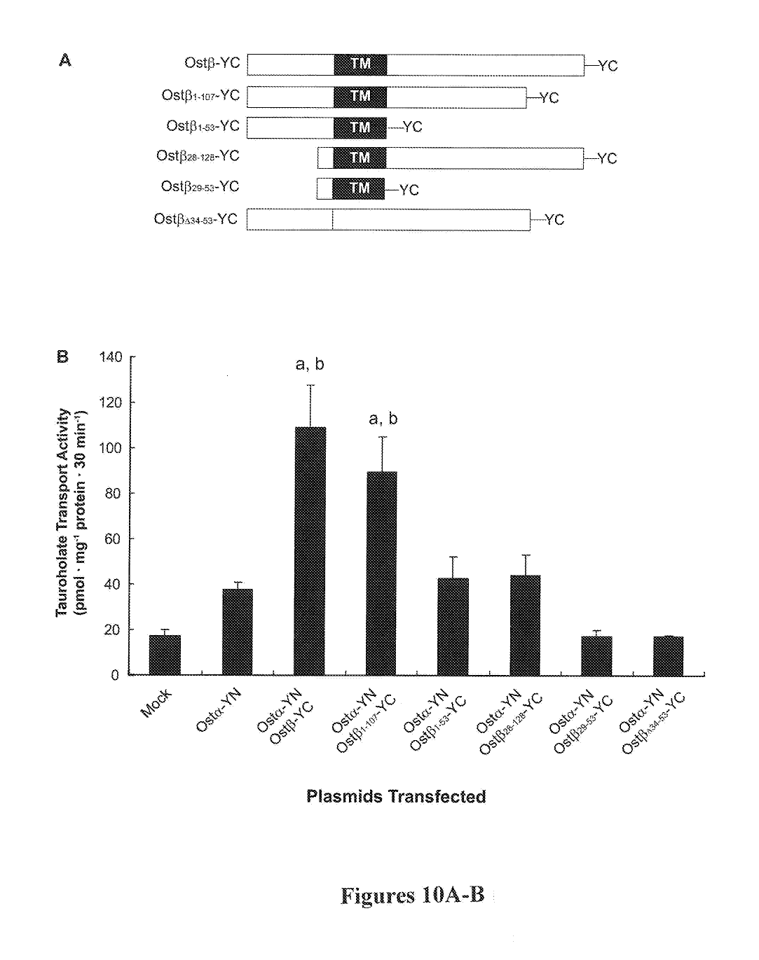

Production of Fluorescent Fusion Proteins. The various truncated Ostβ sequences (i.e., Ostβ₁₋₁₀₇, Ostβ₁₋₅₃, Ostβ₂₈₋₁₂₈, and Ostβ₂₉₋₅₃) were generated by PCR. Primers used for mutagenesis and cloning are shown in Table 1 below. PCR products were purified by gel electrophoresis, digested with KpnI and EcoRI, and then ligated into the pBiFCC155 plasmid to produce Ostβ₁₋₁₀₇-YC, Ostβ₁₋₅₃-YC, Ostβ₂₈₋₁₂₈-YC, Ostβ₂₉₋₅₃-YC (FIG. 10A). The Ostβ sequence lacking the TM domain (Ostβ_{Δ34-53}-YC) was constructed by a two-step approach. The Ostβ54 forward primer was designed with a HindIII restriction site such that the Ostβ (54-128) PCR fragment included a HindIII site on its 5′ end. After subcloning into the pBiFCC155 plasmid using KpnI and EcoRI sites, the plasmid was cut with EcoRI and HindIII, and the Ostβ (1-33) PCR product with the same restriction enzyme sites was inserted. Ostα_{C7/S7}, Ostα_{RRK/AAA}, and Ostβ_{RNR/AAA} mutants were generated using the QuikChange® II XL site-directed mutagenesis kit (200521) from Stratagene (La Jolla, Calif.), and the primers used are shown in Table 1. All constructs were verified by enzyme digestion and sequencing prior to use.

TABLE 1
PCR Primers Used for the Generation of the Various Ostα and Ostβ Constructs

| | | SEQ | Restriction |
|---|---|---|---|
| Primer Name | Sequence (5′ . . . 3′) | ID NO: | Sites |
| Ostβ1F | GGGGAATTCGGATGGACCACAGTGCAGAGAAA | 102 | EcoRI |
| Ostβ28F | GGGGAATTCGGGCAGAAGATGCGGCTCCT | 103 | EcoRI |
| Ostβ29F | CCCGAATTCGGATGGAAGATGCGGCTCCTTGG | 104 | EcoRI |
| Ostβ54F | GGGGAATTCGGATGAAGCTTAGAAGGAGCATCCTGGCAAACAGAAATCGA | 105 | EcoRI |
| Ostβ33R | GGGAAGCTTAGGAGCCGCATCTTCTGCACG | 106 | HindIII |
| Ostβ53R | GGGGGTACCCAGGAGGAACATGCTTG | 107 | KpnI |
| Ostβ107R | GGGGGTACCTTCCCCCGGGGCCAAGTC | 108 | KpnI |
| Ostβ128R | CCCGGTACCGCTCTCTGTTTCCTGTGGGTC | 109 | KpnI |
| Ostα_{RRK/AAA}F | GTGCTGACACGAATGTACTATGCAGCGGCAGACGACAAGGTTGG | 110 | |
| Ostα_{RRK/AAA}R | CCAACCTTGTCGTCTGCCGCTGCATAGTACATTCGTGTCAGCAC | 111 | |
| Ostα_{RNR/AAA}F | GAAGGAGCATCCTGGCAAACGCAGCTGCAAAGAAGCAGCCAC | 112 | |
| Ostα_{RNR/AAA}R | GTGGCTGCTTCTTTGCAGCTGCGTTTGCCAGGATGCTCCTTC | 113 | |
| Ostα_{C7/S7}F | CACACGGGTCCCAGTAGCAGCAGCAGTCCCAGCAGCCCACCTCTCATAC | 114 | |
| Ostα_{C7/S7}R | GTATGAGAGGTGGGCTGCTGGGACTGCTGCTGCTACTGGGACCCGTGTG | 115 | |

The DNA fragments were amplified by standard PCR. Forward primers have (+) orientation, and reverse
primers have (−) orientation except for Ostα_{C7/S7}R. The restriction sites that were introduced for each
subclone are listed. The point mutations constructs were generated by QuikChange ® II XL site-directed
mutagenesis kit (Stratagene No. 20052). The sequences that were mutated into alanine or serine are
shown in bold.

GFP tagged constructs were also made by a two-step ligation. Mouse Ostα, Ostα_{RRK/AAA}, Ostβ, and Ostβ_{RNR/AAA} sequences flanked with EcoRV (5′) and XhoI (3′) were amplified by PCR, and an AgeI site was introduced near the 3′ end and ligated into pcDNA3.1/Hygro+ vector (Invitrogen, Carlsbad, Calif.). The ppluGFP2 sequence flanked with AgeI (5′) and XhoI (3′) was amplified by PCR and after digestion inserted into the pcDNA3.1/Hygro+ vector with wild type or mutant Ostα or Ostβ.

Co-Immunoprecipitation (Co-IP) and Western Blotting. Immunoprecipitation was performed as described supra. For Western blotting, HEK293 cells were washed with ice-cold PBS and then scraped into ice-cold PBS containing protease inhibitor cocktail. The cells were pelleted at 10,000×g at 4° C. and stored at −70° C. Cell extracts were prepared by lysing the cell pellets in whole cell lysis buffer (25 mM Tris-HCl, pH 7.4, 300 mM NaCl, 1 mM CaCl₂, 1% Triton X-100, 1 mM phenylmethylsulfonyl fluoride, 0.625% (vol/vol) protease inhibitor cocktail, and 10 mM EDTA) via repeated aspiration through a 25-gauge needle. The samples were centrifuged at 10,000×g for 2 mM at 4° C., and aliquots of cell supernatants were stored at −70° C. Samples (40-60 μg) were diluted into SDS sample buffer with β-mercaptoethanol, incubated at 37° C. for 30 mM, and separated on a 4-20% Tris-SDS-PAGE gel. The separated polypeptides were electrotransferred onto a PVDF membrane for 90 mM at 95 volts using a wet transfer apparatus. The membranes were blocked overnight in 5% milk in TBST at 4° C. Antibodies were used at the following dilutions: anti-FLAG (F3165, Sigma-Aldrich), anti-c-myc (9E10, Sigma-Aldrich) or anti-HA (12CA5, Roche, Switzerland), and horseradish peroxide-conjugated anti-mouse secondary antibody (Sigma-Aldrich) at 1:3000. Antibody binding was then detected using an enhanced chemiluminescence technique.

Statistical Analyses. Data were evaluated using one-way ANOVA followed by Bonferroni's multiple comparison test or the two-tailed unpaired Student's t test and are given as mean±SEM. Differences were considered statistically significant at p<0.05.

### Example 7

### Ostβ Plays a Direct Role in the Transport of Bile Acids

To examine whether Ostβ is required for the formation of the functional transport unit at the plasma membrane, various Ostβ mutants were generated and examined for their ability to support taurocholate transport activity in transiently transfected HEK293 cells. As described supra, cells co-expressing wild type Ostα-YN and Ostβ-YC demonstrated significant taurocholate uptake activity (FIG. 10B). Deletion of the C-terminal 21 amino acids of Ostβ (Ostβ₁₋₁₀₇-YC) had only small effects on transport activity, whereas further truncation of the C-terminus to Ostβ₁₋₅₃-YC markedly decreased the transport rate (FIG. 10B). Removal of the N-terminal 27 amino acids (Ostβ₂₈₋₁₂₈-YC) also abolished taurocholate transport activity, indicating that the N-terminal 27 amino acids as well as amino acids 54-107 are required for functional activity (FIG. 10B).

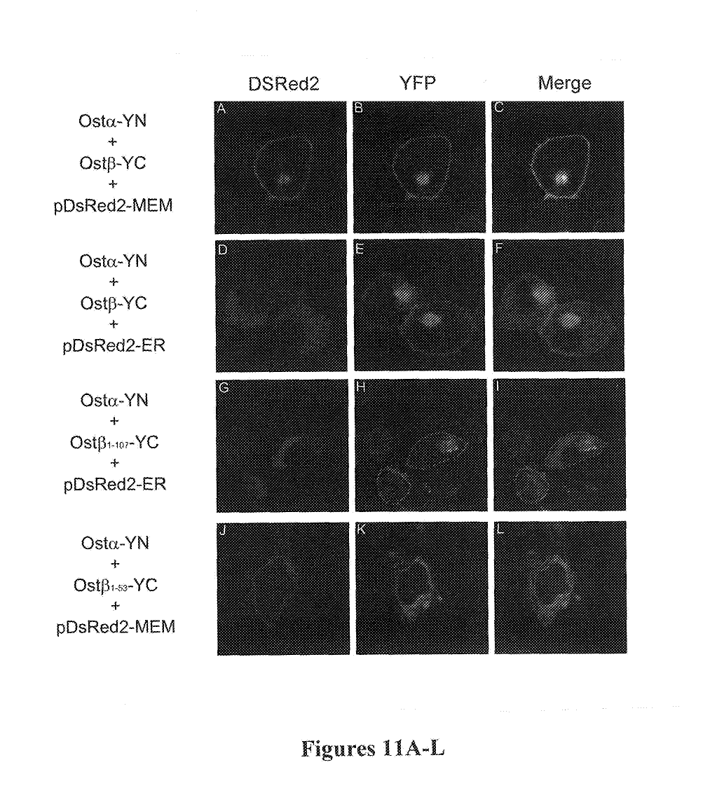

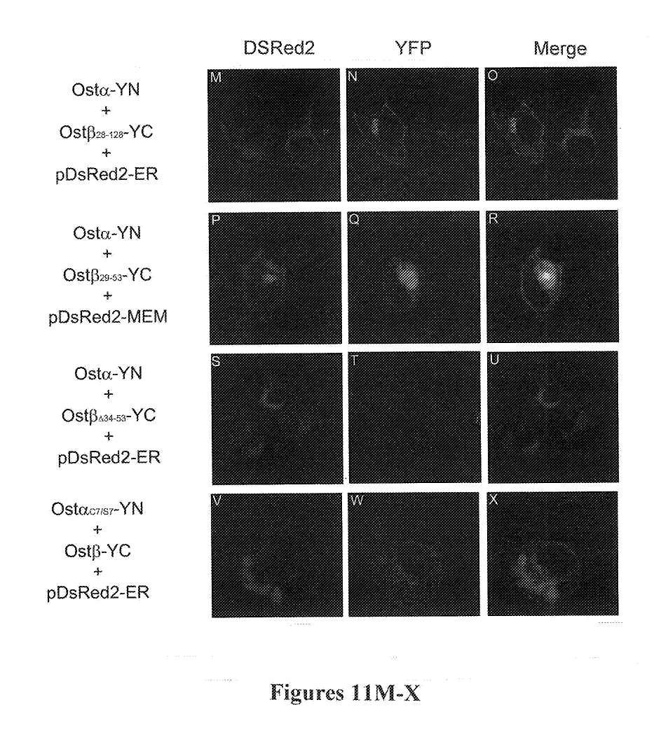

To investigate the mechanism of transport activity loss, bimolecular fluorescence complementation (BiFC) (Hu et al., “Visualization of Interactions Among bZIP and Rel Family Proteins in Living Cells Using Bimolecular Fluorescence Complementation,” Mol. Cell. 9(4):789-98 (2002); Kerppola, “Design and Implementation of Bimolecular Fluorescence Complementation (BiFC) Assays for the Visualization of Protein Interactions in Living Cells,” Nat. Protoc. 1(3):1278-86 (2006); and Kerppola, “Complementary Methods for Studies of Protein Interactions in Living Cells,” Nat. Methods 3(12):969-71 (2006), which are hereby incorporated by reference in their entirety) was used to establish the ability of these Ostβ mutants to heterodimerize with Ostα-YN, and to facilitate delivery of the resulting complexes to the plasma membrane. For these experiments, either pDsRed2-ER or pDsRed2-MEM was co-transfected as ER or plasma membrane markers, respectively. In agreement with previous results, the wild type Ostα-YN and Ostβ-YC proteins heterodimerized within the cell, and the resulting complexes were correctly targeted to the plasma membrane (FIGS. 11B and E). Deletion of the C-terminal 21 amino acids had no affect on the subcellular localization of the complex (FIG. 11H), supporting the lack of effect of this mutation on taurocholate transport activity (FIG. 10B). On the other hand, although Ostβ₁₋₅₃-YC was able to heterodimerize with Ostα-YN, the YFP signal was detected mainly intracellularly (FIG. 11K). Thus, amino acids 1-53 of Ostβ most likely contain the heterodimerization domain or domains, and amino acids 54-107 most likely contain plasma membrane targeting information.

Interestingly, the mutant in which the N-terminal 27 amino acids of Ostβ were deleted (i.e., Ostβ₂₈₋₁₂₈-YC) was able to heterodimerize with Ostα-YN and localized correctly at the plasma membrane (FIG. 11N), yet the resulting complex lacked transport activity (FIG. 10B), indicating that the N-terminal 27 amino acids are important for functional activity. Thus, Ostβ is required not only for delivery of Ostα to the plasma membrane, but it is also required for formation of the functional transport unit.

### Example 8

### A Region Encompassing the Transmembrane Domain of Ostβ is Necessary and Sufficient for Binding to Ostα

As noted above, Ostβ₁₋₅₃-YC was able to heterodimerize with Ostα-YN (FIG. 11N), and thus the heterodimerization domain or domains are likely present in amino acids 1-53 of Ostβ. This region includes the Ostβ transmembrane domain, which is part of the most evolutionarily conserved region of the protein (FIG. 2), and may therefore be a site of interaction. As illustrated in FIG. 2, a comparison of the predicted Ostβ amino acid sequences among different species reveals that the TM domain and specific amino acids in close proximity (20 amino acid residues toward the N terminal side of the TM domain) to this membrane helix are the most conserved residues. Conserved amino acid residues across these species include a glutamate-aspartate sequence near the N-terminus of the TM helix, a tryptophan at the predicted start of the TM domain, and an arginine located 8 amino acids from the C-terminus of the TM domain (FIG. 2A).

To investigate whether the Ostβ TM helix and the nearby amino acids are required for heterodimerization with Ostα, an Ostβ construct was made that lacked amino acids 34-53 (Ostβ_{Δ34-53}-YC) and one that consisted mainly of the Ostβ ™ domain and some upstream amino acids (Ostβ₂₉₋₅₃-YC). The latter construct contained the 20 amino acids that are predicted to form the TM helix, along with 5 N-terminal amino acids of the mouse sequence (i.e., EDAAP, residues 29-33 of SEQ ID No: 9) of the mouse sequence (FIG. 2A). As noted above, the two negatively charged amino acids in this sequence are highly conserved among species (FIG. 2A). These anionic amino acids were also preserved in this construct to facilitate properly oriented insertion of the peptide within the membrane (Tarasova et al., “Inhibition of G-protein-coupled Receptor Function by Disruption of Transmembrane Domain Interactions,” J. Biol. Chem. 274(49):34911-5 (1999); and Tarasova et al., “Transmembrane Inhibitors of P-glycoprotein, an ABC Transporter,” J. Med. Chem. 48(11):3768-75 (2005), which are hereby incorporated by reference in their entirety). The results demonstrate neither Ostβ_{Δ34-53}-YC nor Ostβ₂₉₋₅₃-YC was able to generate taurocholate transport activity when co-expressed with Ostα-YN (FIG. 10B). Interestingly, Ostβ₂₉₋₅₃-YC was able to heterodimerize with Ostα-YN (FIG. 11Q), but the resulting complex was retained intracellularly and co-localized with the ER marker (FIG. 11R). These observations are in agreement with those in FIG. 11K for the Ostβ₁₋₅₃-YC construct. In contrast, no fluorescence was detected when Ostβ_{Δ34-53}-YC was co-expressed with Ostα-YN (FIG. 11T), demonstrating that deletion of the TM domain abolishes its ability to interact with Ostα-YN. These results are consistent with the lack of taurocholate transport activity (FIG. 10B). Western blot analysis indicated both Ostα-YN and Ostβ_{Δ34-53}-YC proteins were present, although at very low levels under these experimental conditions, consistent with previous findings demonstrating that non-heterodimerized proteins are more rapidly degraded (Example 6). Taken together, these observations indicate that this region encompassing the TM domain of Ostβ is necessary and sufficient for binding to Ostα; however, the region by itself is not sufficient for delivery of the heterodimeric complex to the plasma membrane, and thus the resulting complex is unable to mediate taurocholate transport in intact cells (FIG. 10B).

### Example 9

### The TM Region of Ostβ Competes with the Wild Type Ostβ and Decreases Taurocholate Transport Activity When Co-expressed in HEK293 Cells

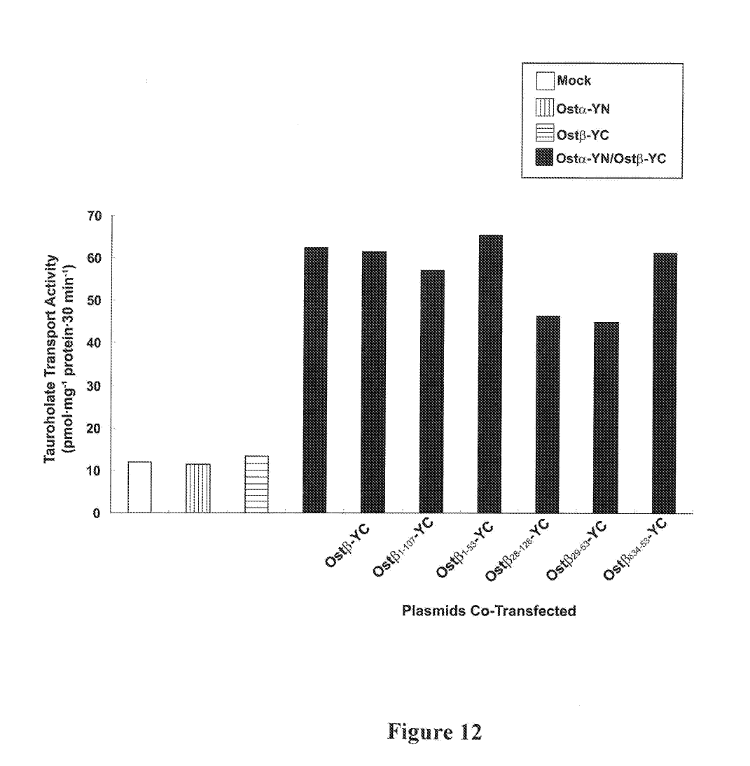

As noted in Example 8, the TM region of Ostβ (amino acids 29-53 of the SEQ ID NO: 9) alone was sufficient for interaction with Ostα, but the resulting complex was retained in the ER. This observation is consistent with the expection that a peptide derived from this TM domain should be able to compete with full length Ostβ for Ostα bonding and trap newly synthesized Ostα in the ER. To test this possibility, HEK293 cells were co-transfected the various truncated Ostβ constructs, along with Ostα-YN and Ostβ-YC. In this experiment, 0.5 μg of wild type Ostα and Ostβ construct were used, along with 1 μg the truncated Ostβ plasmids (FIG. 12). As previously shown, Ostα-YN or Ostβ-YC alone are unable to generate significant transport activity (FIG. 12, lanes 1-3), whereas expression of wild type Ostα-YN and Ostβ-YC resulted in enhanced [³H]taurocholate transport (FIG. 12, lane 4). The only two Ostβ constructs that inhibited transport were Ostβ₂₈₋₁₂₈-YC, and Ost₂₉₋₅₃-YC. The Ostβ₂₉₋₅₃-YC construct, which corresponds to the Ostβ transmembrane domain and amino acid region upstream, decreased transport activity ˜30% (FIG. 12).

### Example 10

### The Conserved Cysteine-Rich Region of Ostα is Not Required for Dimerization or Trafficking, But is Required for Transport Function

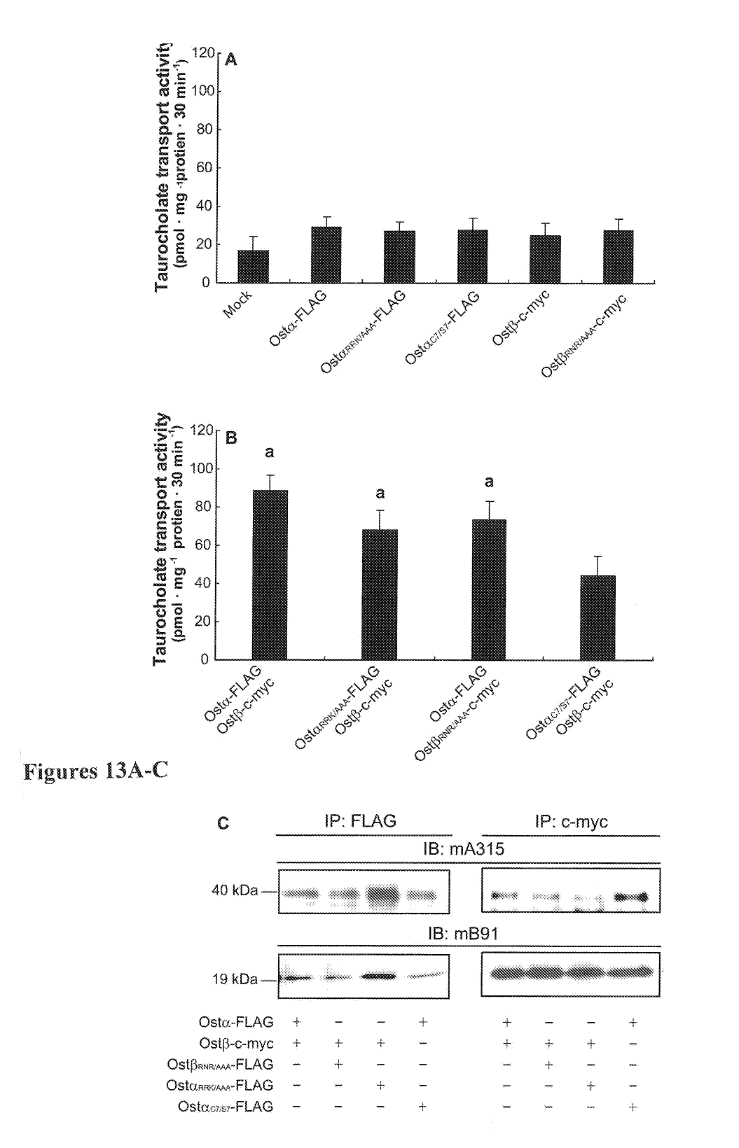

For Ostα, an unusual, highly conserved stretch of 6-7 cysteine residues is found in the predicted cytosolic loop between TM domains 3 and 4 (Seward et al., “Functional Complementation Between a Novel Mammalian Polygenic Transport Complex and an Evolutionarily Ancient Organic Solute Transporter, Ostα-Ostβ,” J. Biol. Chem. 278(30):27473-82 (2003), which is hereby incorporated by reference in its entirety). To investigate whether this region is involved in dimerization, trafficking, or function, all seven cysteine residues were substituted with serine (Ostα_{C7/S7}). Co-immunoprecipitation experiments revealed the mutant was able to interact with wild type Ostβ when co-expressed in HEK293 cells (FIG. 13C). In addition, BiFC analysis showed that Ostα_{C7/S7}-YN was able to heterodimerize with Ostβ-YC and this complex localized correctly to the plasma membrane (FIGS. 11V-X); however, taurocholate transport activity was significantly lower (FIG. 13B), indicating that this mutation does not impair heterodimerization or trafficking but leads to a decrease in transport activity. Thus, the cysteine-rich region may be involved in substrate binding or translocation, or regulation of protein structure or post-translational modification.

### Example 11

### Replacement of the RRK/RNR Sequences Does Not Affect Transport Activity

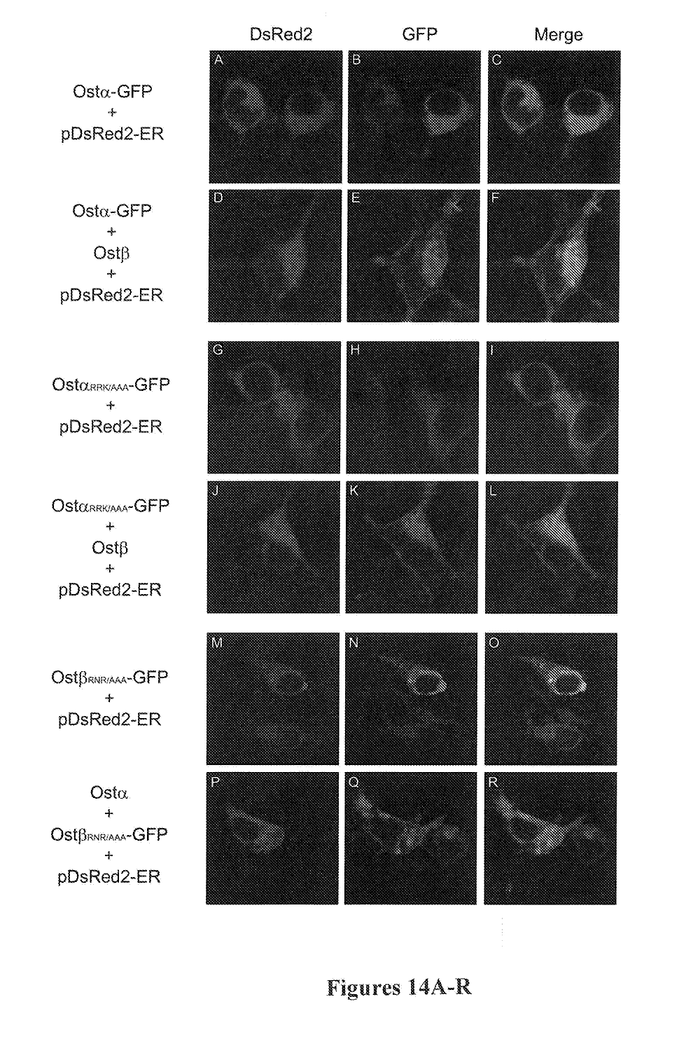

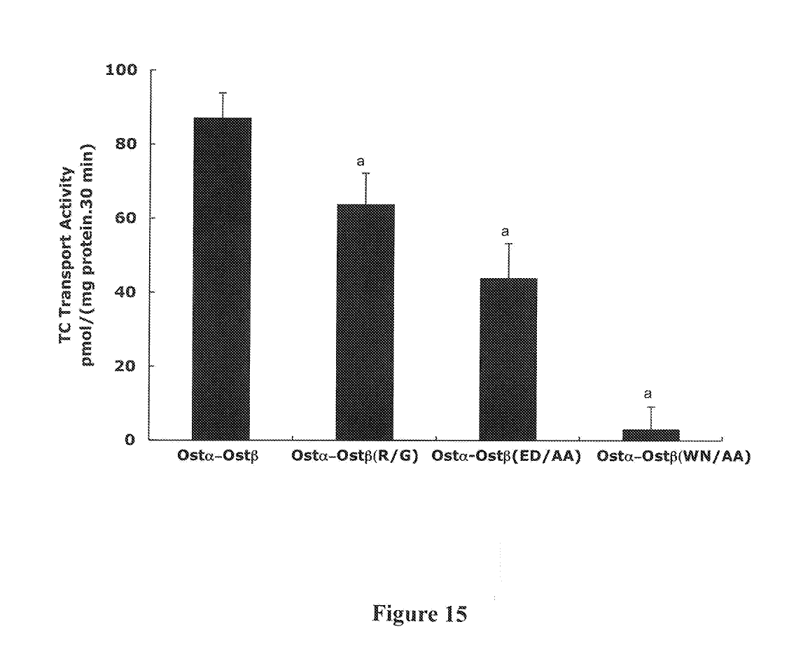

Mouse Ostα and Ostβ proteins both have putative ER retention sequences (RRK and RNR, respectively) in their C-terminus (Seward et al., “Functional Complementation Between a Novel Mammalian Polygenic Transport Complex and an Evolutionarily Ancient Organic Solute Transporter, Ostα-Ostβ,” J. Biol. Chem. 278(30):27473-82 (2003); and Li et al., “Heterodimerization, Trafficking and Membrane Topology of the Two Proteins, Ostα and Ostβ, That Constitute the Organic Solute and Steroid Transporter,” Biochem. J. 407(3):363-72 (2007), which is hereby incorporated by reference in its entirety). Such sequences in hetero-oligomeric proteins may function as retention or retrieval signals that must be masked before the corresponding protein complexes can be transported from the ER (Ellgaard et al., “Setting the Standards: Quality Control in the Secretory Pathway,” Science 286(5446):1882-8 (1999); Ma et al., “ER Transport Signals and Trafficking of Potassium Channels and Receptors,” Curr. Opin. Neurobiol. 12(3):287-92 (2002); Margeta-Mitrovic et al., “A Trafficking Checkpoint Controls GABA(B) Receptor Heterodimerization,” Neuron 27(1):97-106 (2000); Michelsen et al., “Hide and Run. Arginine-based Endoplasmic-reticulum-sorting Motifs in the Assembly of Heteromultimeric Membrane Proteins,” EMBO Rep. 6(8):717-22 (2005); Zerangue et al., “A New ER Trafficking Signal Regulates the Subunit Stoichiometry of Plasma Membrane K(ATP) Channels,” Neuron 22(3):537-48 (1999), which are hereby incorporated by reference in their entirety). To examine whether these sequences are membrane sorting signal motifs and are involved in transport activity, mutations of the RRK and RNR sequences were made. Ostα_{RRK/AAA}-GFP and Ostβ_{RNR/AAA}-GFP constructs were created to investigate whether these individual proteins can be delivered to the cell surface without the aid of their dimerization partner. If the RRK and RNR regions function as ER retrieval signals, mutation of these regions may result in direcTrafficking to the plasma membrane. However, neither Ostα_{RRK/AAA}-GFP nor Ostβ_{RNR/AAA}-GFP was able to reach the plasma membrane without co-expression of their heterodimerization partner (FIGS. 14G-L and M-R). Likewise, when unmodified Ostα-GFP was expressed alone, the GFP fluorescence largely co-localized with the ER marker (FIGS. 14A-C), but upon co-expression with wild type Ostβ, fluorescence was also observed on the cell surface (FIGS. 14D-F). The same shift in subcellular localization was observed in HEK293 cells expressing Ostβ-GFP in the absence and presence of wild type Ostα, when Ostα_{RRK/AAA}-GFP was paired with wild type Ostβ (FIGS. 14J-L), and when Ostβ_{RNR/AAA}-GFP was paired with wild type Ostα (FIGS. 14P-R). Heterodimerization of the RRK and RNR mutants was also examined by co-immunoprecipitation, and the replacement of these sequences did not prevent interaction between the two subunits (FIG. 13C).

Taurocholate transport assays revealed that transport activity was decreased either Ostα_{RRK/AAA}-FLAG or Ostβ_{RNR/AAA}-c-myc were expressed alone (FIG. 13A). However, upon co-expression with their wild type partner, they generated approximately the same level of transport activity as the wild type counterparts (FIG. 13B). Taken together, these results suggest that the RNR and RRK sequences do not function as retrieval signals in Ostα and Ostβ and are not critical for heterodimerization or function.

### Discussion of Examples 7-11

Ostα-Ostβ is an unusual organic solute transporter composed of two distinct subunits which interact to generate the functional transport unit. The results of Examples 7-11 indicate that the Ostβ subunit is not just a chaperone for delivery of the larger Ostα subunit to the plasma membrane, but is also required to generate the functional plasma membrane transporter. These results also demonstrate that the Ostβ ™ domain is necessary and sufficient for heterodimerization with Ostα, and thus they demonstrate one approach for blocking Ostα-Ostβ functional activity. In addition, these results of Examples 7-11 provide important insights into other amino acid regions involved in Costa-Ostβ trafficking and transport activity.

Even though the overall Ostβ amino acid identity is low among species, the Ostβ TM region from evolutionarily divergent species exhibits higher amino acid identity (>40%) (FIGS. 2A-B). This indicates that the TM domain may be a key element for heterodimerization or transport activity. The results of Examples 7-11 confirm this belief by demonstrating the requirement of the TM region for formation of an obligate heteromeric complex with Ostα. When this part of the polypeptide was deleted (Ostβ_{Δ34-53}-YC), no evidence for an interaction with Ostαwas found, and no transport activity was detected. On the other hand, a 25-amino acid peptide sequence consisting mainly of the TM domain and some upstream amino acids (Ostβ₂₉₋₅₃-YC) was able to heterodimerize with Ostα-YN although the resulting complex failed to reach the plasma membrane. This peptide, therefore, represents a new class of peptide inhibitors of Ostα-Ostβ activity.

Interestingly, comparable findings have been reported for another class of heteromeric proteins with similar predicted membrane architecture as Ostα-Ostβ, namely the G-protein coupled receptor-receptor activity-modifying protein (GPCR-RAMP) complexes (Morfis et al., “RAMPs: 5 Years on, Where to Now?,” Trends Pharmacol. Sci. 24(11):596-601 (2003); Hay et al., “GPCR Modulation by RAMPs,” Pharmacol. Ther. 109(1-2):173-97 (2006); Bockaert et al., “Molecular Tinkering of G Protein-coupled Receptors: An Evolutionary Success,” Embo. J 18(7):1723-9 (1999), which are hereby incorporated by reference in their entirety). Recent studies have shown the single TM domain of the RAMP proteins is required for their heterodimerization with the 7-TM domain GPCR proteins and formation of fully functional GPCR-RAMP complexes (Steiner et al., “The Transmembrane Domain of Receptor-activity-modifying Protein 1 is Essential for the Functional Expression of a Calcitonin Gene-Related Peptide Receptor,” Biochemistry 41(38):11398-404 (2002), which is hereby incorporated by reference in its entirety). Not surprisingly, one or more of the 7 ™ domains in the GPCRs are also critical for their heterodimerization with the RAMP proteins and for GPCR hetero- and homo-oligomerization (Carrillo et al., “Multiple Interactions Between Transmembrane Helices Generate the Oligomeric Alpha1b-adrenoceptor,” Mol. Pharmacol. 66(5):1123-37 (2004); Hebert et al., “A Peptide Derived From A Beta2-adrenergic Receptor Transmembrane Domain Inhibits Both Receptor Dimerization and Activation,” J. Biol. Chem. 271(27):16384-92 (1996); Lee et al., “D2 Dopamine Receptor Homodimerization is Mediated by Multiple Sites of Interaction, Including an Intermolecular Interaction Involving Transmembrane Domain 4,” Biochemistry 42(37):11023-31 (2003); Overton et al., “The Extracellular N-terminal Domain and Transmembrane Domains 1 and 2 Mediate Oligomerization of a Yeast G protein-coupled Receptor,” J. Biol. Chem. 277(44):41463-72 (2002); and Ng et al., “Dopamine D2 Receptor Dimers and Receptor-blocking Peptides,” Biochem. Biophys. Res. Commun. 227(1):200-4 (1996), which are hereby incorporated by reference in their entirety). These findings with the GPCR-RAMP complexes in combination with the above-noted results demonstrating the requirement of the Ostβ TM domain, indicate that one or more of the 7 ™ domains in Ostα may be required for interaction with the Ostβ TM domain (FIG. 11) and for Ostα homo-dimerization.

Although the present results demonstrate that the TM region of Ostβ is necessary and sufficient for heterodimeriztion with Ostα, other sites may also stabilize or contribute to this interaction. For example, Sun et al. “Protein-Protein Interactions and Membrane Localization of the Human Organic Solute Transporter,” Am. J. Physiol. 292(6):G1586-93 (2007), which is hereby incorporated by reference in its entirety suggested that the 50 amino acids on the N-terminus of human Ostα may contain information for the assembly of the heterodimer and for trafficking to the plasma membrane, but the mechanism was not identified.

Another major finding of the preceding Examples is Ostβ's direct participation in generating transport activity. Ostβ lacking the N-terminal 27 amino acids was able to heterodimerize with Ostα, and the resulting complex was correctly targeted to the plasma membrane but failed to generate transport activity. These observations indicate that Ostβ not only modulates Ostα post-translational modifications, membrane trafficking, and turnover rate (Li et al., “Heterodimerization, Trafficking and Membrane Topology of the Two Proteins, Ost alpha and Ost beta, That Constitute the Organic Solute and Steroid Transporter,” Biochem. J. 407(3):363-72 (2007); and Dawson et al., “The Heteromeric Organic Solute Transporter α-β, Ostα-Ostβ, is an Ileal Basolateral Bile Acid Transporter,”J. Biol. Chem. 280(8):6960-8 (2005), which are hereby incorporated by reference in their entirety) but is also important for formation of the functional transport unit. Furthermore, these conclusions on the role of Ostβ in Ostα-Ostβ-mediated functional activity are comparable to reported GPCR-RAMP interactions. The RAMP proteins not only regulate membrane trafficking of certain GPCRs, but they appear to regulate receptor activity and phenotype as well (Hay et al., “GPCR Modulation by RAMPs,” Pharmacol. Ther. 109(1-2):173-97 (2006), which is hereby incorporated by reference in its entirety).

In contrast to N-terminal deletions, Ostβ lacking the C-terminus 21 amino acids was similar to wild type in terms of function and subcellular localization. The further removal of the 75 amino acids in the C-terminus of Ostβ changed the localization of the complex formed with Ostα from the plasma membrane to intracellular sites co-localizing with the ER marker. Thus, amino acids from position 54-107 likely encode plasma membrane targeting information. However, mutation of amino acids 61-63 (i.e., conversion of RNR to AAA) in mouse Ostβ had no effect on heterodimerization, trafficking, or taurocholate transport activity, and thus these three amino acids are probably not critical for these processes.

In addition, the present results identify the cysteine-rich region of Ostα as a determinant of transport activity. When the region was mutated, dimerization and trafficking was unaffected, but transport activity was decreased. Therefore, this site may be involved in substrate binding, translocation, or in stabilizing protein structure.

Of significance, given that heterodimerization of Ostα and Ostβ is required for function, the present findings provide important insight into a potential strategy for preventing plasma membrane delivery of functional transporter. Observing that the Ostβ TM region is a major site of interaction led to the expectation that a peptide derived from the TM domain might be able to compete with Ostβ for binding to Ostα. This was demonstrated with the Ostβ₂₉₋₅₃ peptide, which inhibited trafficking of Ostα to the plasma membrane and abolished transport activity. In addition, because the interaction between Ostα and Ostβ is required for protein stability, the peptide mimetic might accelerate the degradation of any uncomplexed Ostβ proteins, accentuating the inhibitory effect.

Examples 7-11 therefore identify a class of peptides that may be useful as inhibitors of intestinal bile acid absorption. Because bile acids are required for intestinal lipid absorption, inhibition of Ostα-Ostβ using one or more peptides that mimic the heterodimerization site should diminish both bile acid and lipid absorption, and thus such drugs should be useful in a variety of human conditions related to imbalances in bile acid, sterol, or lipid homeostasis. Dyslipidemia, including hypercholesterolemia and hypertriglyceridemia, is a common and important cluster of risk factors for coronary heart disease, hypertension, hyperinsulinemia and obesity. Obesity, in turn, is associated with an increased risk of many other diseases, including stroke and certain forms of cancer. Treatments of lipid imbalances involve a combination of altered diet and other lifestyle changes, drugs that alter lipid absorption and/or metabolism, and in some cases surgery. The three major pharmacologic approaches for treating dyslipidemias are: 1) Inhibiting cholesterol synthesis with statins (Friesen et al., “The 3-hydroxy-3-methylglutaryl Coenzyme-A (HMG-CoA) Reductases,” Genome Biol. 5:248 (2004), which is hereby incorporated by reference in its entirety); 2) Inhibiting cholesterol absorption with ezetimibe (Davis et al., “Zetia: Inhibition of Niemann-Pick Cl Like 1 (NPC1L1) To Reduce Intestinal Cholesterol Absorption and Treat Hyperlipidemia,” J. Atheroscler. Thromb. 14:99-108 (2007); and Altmann et al., “Niemann-Pick Cl Like 1 Protein Is Critical for Intestinal Cholesterol Absorption,” Science 303:1201-4 (2004), which are hereby incorporated by reference in there entirety), plant stanols/sterols, polyphenols, or with nutraceuticals such as oat bran, psyllium and soy proteins (Patch et al., “Plant Sterols As Dietary Adjuvants in the Reduction of Cardiovascular Risk: Theory and Evidence,” Vasc. Health Risk Manag. 2:157-162 (2006), which is hereby incorporated by reference in its entirety); and 3) Sequestering bile acids in the intestine with non-absorbable resins (Bays et al., “Pharmacotherapy for Dyslipidaemia—Current Therapies and Future Agents,” Expert. Opin. Pharmacother. 4:1901-1938 (2003); Insull, W Jr., “Clinical Utility of Bile Acid Sequestrants in the Treatment of Dyslipidemia: a Scientific Review,” South. Med. J. 99:257-273 (2006); Kobayashi et al., “Prevention and Treatment of Obesity, Insulin Resistance, and Diabetes by Bile Acid-Binding Resin,” Diabetes 56:239-247 (2007); Jacobson et al., “Safety Considerations With Gastrointestinally Active Lipid-Lowering Drugs,” Am. J. Cardiol. 99:47 C-55C (2007); Charlton-Menys et al., “Human Cholesterol Metabolism and Therapeutic Molecules,” Exp. Physiol. 93:27-42 (2008), which are hereby incorporated by reference in their entirety). Adjuvant therapies include: fibrates to increase expression of lipoprotein lipase (Moon et al., “Ezetimibe and Fenofibrate Combination Therapy for Mixed Hyperlipidemia,” Drugs Today 43:35-45 (2007), which is hereby incorporated by reference in its entirety), niacin to raise the levels of high-density lipoprotein cholesterol (HDL-C) (Sanyal et al., “Present-day Uses of Niacin: Effects On Lipid and Non-lipid Parameters,” Expert. Opin. Pharmacother. 8:1711-7 (2007), which is hereby incorporated by reference in its entirety), and drugs such as orlistat that inhibit pancreatic lipase, thereby reducing the digestion and absorption of fat in the small intestine (Drew et al., “Obesity Management: Update On Orlistat,” Vasc. Health Rish Manag. 3:817-21 (2007), which is hereby incorporated by reference in its entirety). Although reduction of plasma cholesterol by statins is effective at diminishing the risk of coronary heart disease, such therapy is often sub-optimal, particularly in patients with reduced LDL receptors (familial hypercholesterolemia), or results in undesirable side-effects such as inflammation. Novel or adjuvant therapies are therefore warranted.

The present Examples reveal another approach that may be used to inhibit intestinal bile acid absorption, namely to prevent the trafficking of the functional Ostα-Ostβ transporter to the enterocyte basolateral plasma membrane. This approach would appear to offer significant advantages over simple competitive inhibitors of intestinal bile acid uptake transporters or over agonists/antagonists of the various transcription factors. First, peptide-based drugs should be much more selective, and thus it should be possible to inhibit Ostα-Ostβ without affecting other transporters, enzymes, or signaling pathways. Second, the Ostα and Ostβ proteins are relatively unstable and are rapidly degraded in the absence of their dimerization partner. Thus, when the peptide drug binds to one protein, the unassociated protein should be more susceptible to degradation, and thus the inhibition of bile acid transport activity may be prolonged and/or heightened. Third, as demonstrated in the Ostα^{-/-} mice, disruption of basolateral bile acid transport disrupts the normal homeostatic control of bile acid biosynthesis, such that bile acid synthesis rate is markedly decreased. Ostα^{-/-} mice have significantly lower hepatic Cyp7a1 expression, a much smaller bile acid pool size, and lower serum cholesterol and triglyceride levels. In addition, fecal cholesterol excretion is 4-fold higher in Ostα^{-/-} mice (Rao et al., “The Organic Solute Transporter {alpha}-{beta}, Ost {alpha}-Ost {beta}, is Essential for Intestinal Bile Acid Transport and Homeostasis,” Proc. Natl. Acad. Sci. USA. 105(13):4965-6 (2008), which is hereby incorporated by reference in its entirety). Thus Ostα-Ostβ inhibition should not be associated with a compensatory increase in bile acid or cholesterol synthesis rate, and this would accentuate the effect of these drugs.

Although peptide-based drugs have some potential drawbacks, including their generally low stability and solubility in biological media and their low membrane permeability (Hamman et al., “Oral Delivery of Peptide Drugs: Barriers and Developments,” BioDrugs 19:165-177 (2005), which is hereby incorporated by reference in its entirety), there are several approaches for overcoming these limitations, including chemical modifications, receptor-targeted delivery mechanisms such as small-molecule peptides, monoclonal antibodies, and specific vectors, as well as non-targeted procedures such as liposomes, nanostructures, and natural and synthetic polymers. In addition, it may be possible to limit metabolism of peptides by co-administration of competitive enzyme inhibitors (Malik et al., “Recent Advances in Protein and Peptide Drug Deliver,” Curr. Drug Deliv. 4:141-51 (2007), which is hereby incorporated by reference in its entirety).

Taken together, these findings suggest a novel pharmacologic approach for treating a variety of human conditions related to imbalances in bile acid or lipid homeostasis.

### Materials and Methods for Examples 12-17

Materials. [³H(G)]Taurocholic acid (2 Ci/mmol), [1,2,6,7-³H(N)]DHEAS (74 Ci/mmol), and [6,7⁻³H(N)]estrone 3-sulfate (46 Ci/mmol) were purchased from PerkinElmer Life and Analytical Sciences, Inc. (Boston, Mass.).

Clinical chemistry analysis. Female mice that had been fasted overnight were anesthetized with pentobarbital sodium, 50 mg/kg, ip, and blood was collected by cardiac puncture. Serum was immediately separated by centrifugation for 15 min at 1,700 g at 4° C., and was frozen at −20° C. Clinical chemistry analysis was performed by Charles River Laboratories (Wilmington Mass.).

Bile acid analysis. To assess total bile acid pool size, the liver, gall bladder, bile duct and small intestine were removed from anesthetized Ostα^{+/+} and Ostα^{-/-} female mice that had been fasted overnight. Feces were collected over a 24 h period from Ostα^{+/+} Ostα^{-/-} female mice that had unrestricted access to their normal diet. The tissues and feces were homogenized in 10-volumes of 10N NaOH using an Ultra-Turrax homogenizer (Janke & Kunkel IKA Labortechnik, Staufen im Breisgau, Germany). The insoluble material was removed through centrifugation at 1,700 g, and fecal lysates were also passed through a 0.45 μm nitrocellulose filter (Millipore, Billerica, Mass.). The samples were neutralized with HCl prior to analysis of total bile acids using an enzymatic assay (Mashige et al., “Direct Spectrophotometry of Total Bile Acids in Serum,” Clin. Chem. 27: 1352-1356 (1981), which is hereby incorporated by reference in its entirety).

Total mRNA isolation from mouse tissues. Wild type and Ostα^{-/-} female mice that had been fed ad libitum were anesthetized with pentobarbital sodium, 50 mg/kg, ip. Liver, kidney, and small intestine were collected. The small intestine was divided into three equal sections along the cephalocaudal axis, and total mRNA was isolated using Rneasy Midi Kit (Qiagen).

Real-time quantitative reverse-transcriptase PCR analyses. Gene-specific oligonucleotide primers (Table 2, below) were designed using Primer Express® 1.5 (Applied Biosystems, Foster City, Calif.). Relative gene expression was determined as described supra. Expression levels were quantified as a ratio to mouse β-actin within each sample and reported as percent of control.

TABLE 2
Primer Sequences Used for Real Time RT-PCR Analysis

| Primer | | | SEQ | Tₘ(° C.)/ |
|---|---|---|---|---|
| Pair | Forward Sequence (5′-3′) | Reverse Sequence (5′-3′) | ID NOS: | Size (bp) |
| Ntcp | GCCACACTATGTACCCTACGTC | TTTAGTCGGAAGAGAGCAGAGA | 116, 117 | 62/201 |
| Asbt | GCACAAGCAGTGATGGATAAC | CCTAGGAACTTGTGGACTTCC | 118, 119 | 62/191 |
| Shp1 | AAGATACTAACCATGAGCTCCG | GTCTTGGCTAGGACATCCAAG | 120, 121 | 62/218 |
| Fgf15 | CAGTCTGTGTCAGATGAAGATCC | GATGGCAATCGTCTTCAGAG | 122, 123 | 60/201 |
| Fxr | TCCGGACATTCAACCATCAC | TCACTGCACATCCCAGATCTC | 124, 125 | 55/101 |
| Cyp7a1 | GACATGGAGAAGGCTAAGACG | CCAAGTAAATGGCATTCCCT | 126, 127 | 62/193 |
| Bsep | TGGTAGAGAAGAGGCGACAAT | TGAGGTAGCCATGTCCAGAA | 128, 129 | 62/208 |
| Mrp2 | TATAAATCTCAGTGGCGGTCAG | GAAGTGAATGCCATGTGTAACC | 130, 131 | 62/199 |
| Mrp3 | TGAAGACTGCACCGTACTGAC | AGAAACCCTTGGAATGCATC | 131, 133 | 62/191 |
| Ostβ | AGATGCGGCTCCTTGGAATTA | TGGCAGAAAGACAAGTGATG | 134, 135 | 60/277 |
| β-actin | ACCCTGTGCTGCTCACCGA | CTGGATGGCTACGTACATGGCT | 136, 137 | 59/110 |

Genbank Accession Nos: Ntcp (NM_011387); Asbt (NM_011388); Shp1 (NM_011850);
Fgf15 (NM_008003); Fxr (NM_009108); Cyp7a1 (NM_007824); Bsep (NM_021022); Mrp2
(NM_013806); Mrp3 (NM_029600); Ostβ (NM_178933); β-actin (NM_007393), each of which
is hereby incorporated by reference in its entirety.

Intraileal and intraperitoneal administration of [³H]estrone 3-sulfate, [³H]DHEAS, or [³H]taurocholic acid to anesthetized mice. Mice that had been fed ad libitum were anesthetized by intraperitoneal administration of pentobarbital sodium (50 mg/kg) and additional anesthetic was administered as required for the duration of the experiment. For the intraileal injection, a median laparotomy was performed and 100 μl of 50 μM [³H]estrone 3-sulfate (1 μCi/100 μl) or 100 μM [³H]taurocholic acid (1 μCi/100 μl) was injected using a 27 g needle directly into the ileum (approximately 3 cm above the junction between small intestine and cecum). The experiment was terminated 15 min after [³H]taurocholic acid administration and 30 min after [³H]estrone 3-sulfate administration. [³H]Estrone 3-sulfate (200 μl of 500 μM (1 μCi/200 μl), or 400 μl of 1 mM (1 μCi/400 μl)) and [³H]DHEAS (400 μl of 1 mM; 1 μCi/400 μl) were also administered intraperitoneally and 2 h later tissues were collected. At the end of the experiments, 150-300 μl of blood was collected from the abdominal aorta in a tared 1 ml syringe containing 50 μl of heparin (1000 U/ml). The small intestine was removed and segmented into 3 parts, where the first third after the stomach comprised the duodenum, the middle third the jejunum, and the section before the ileo-ceco-colic junction comprised the ileum. Colon, liver, gallbladder, kidney and the urinary bladder were also removed and weighed. The contents of the small intestine, colon, gallbladder and urinary bladder were included in the analysis. For the liver, approximately 0.2-0.4 g of tissue was taken for analysis of radioisotope content. Tissue and blood samples were placed in tared 20-ml glass vials, and Solvable (Perkin-Elmer, Life and Analytical Sciences, Boston, Mass.) was added to each vial (1.0 ml/0.1 g tissue or 2.0 ml/1.0 ml blood), which was then heated to 60° C. for 2 to 3 h. The vials were allowed to cool to room temperature, and 0.1 ml of 0.1 M EDTA/1.0 ml of Solvable was added to each vial of blood. H₂O₂ (30%) was then added in 0.1-ml aliquots (0.3 ml/1.0 ml Solvable for blood or 0.1 ml/1.0 ml Solvable for tissues). After standing at room temperature for 15 to 30 min, the vials were capped tightly and heated to 60° C. for 1 h and allowed to cool back to room temperature. After the addition of scintillation fluid (4 ml of Opti-Fluor to 200 μl of each sample) (Perkin-Elmer, Life and Analytical Sciences, Boston, Mass.), the samples were allowed to stand for at least 2 h at room temperature before counting. Samples were counted in a Beckman LS 6500 scintillation counter (Beckman Coulter, Fullerton, Calif.). Total blood volume was estimated as 6% of body weight.

Statistical Analyses. Data are given as means±SEM. Mean values were considered to be significantly different, when p<0.05 by use of a one-way ANOVA followed by Bonferroni's multiple comparison test or a Student's t-Test where applicable.

### Example 12

### Ostα-Deficient Mice are Viable and Fertile, But Exhibit Growth Retardation and Have Small Intestine Hypertrophy

As described infra, a conventional gene targeting approach was used to replace exons 3-9 of Ostα with a neomycin-containing cassette in the C57BL/6 background to generate Ostα^{-/-} mice. Western blotting confirmed that these mice lack Ostα protein, but interestingly, these animals also lack the obligate heterodimerization partner of the transporter, namely Ostβ protein, indicating that Ostβ is not stable in the absence of Ostα (Example 6, FIG. 9B).

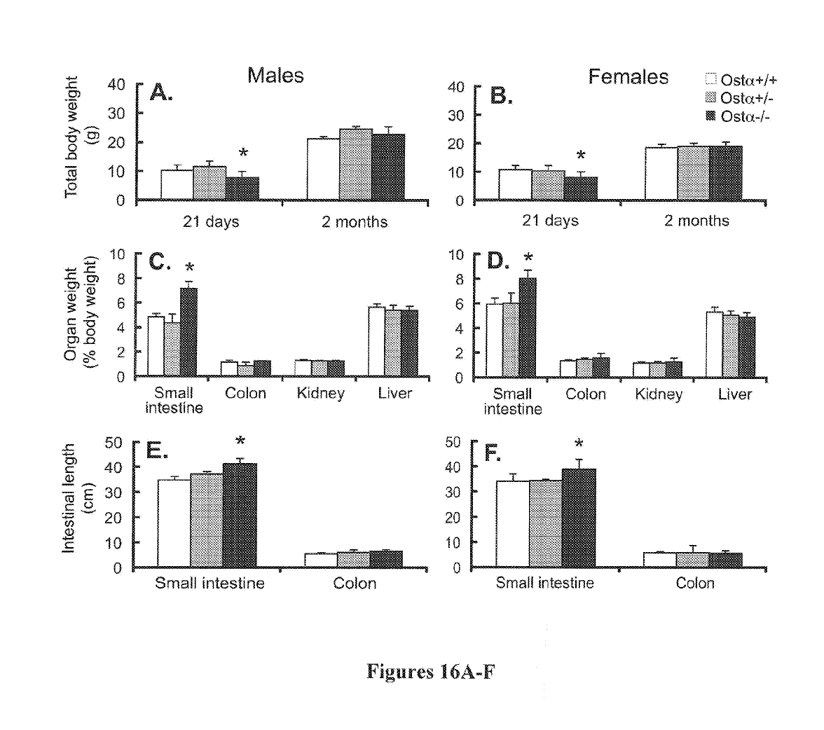

The Ostα^{-/-} mice were found to be viable and fertile, and matings between heterozygous animals produced the predicted Mendelian distribution of genotypes. Ostα^{-/-} mice exhibited no gross abnormalities up to 1.8 years of age, and were able to thrive on normal rodent laboratory chow. However, pre-weanling Ostα^{-/-} mice of both sexes were about 25% smaller than wild-type or heterozygous (Ostα^{+/-}) littermates, although this difference in weight disappeared shortly after weaning (FIGS. 16A and B). Analysis of organ size revealed that the small intestine was heavier and longer in the Ostα^{-/-} mice, suggesting intestinal hypertrophy, whereas the other organs analyzed were similar in weight (FIGS. 16C-F). No gross pathology was noted in the small intestine of Ostα^{-/- mice.}

### Example 13

### Bile Acid Pool Size and Serum Levels Are Markedly Lower in Ostα^{-/-} Mice, But Fecal Bile Acid Excretion is Unchanged

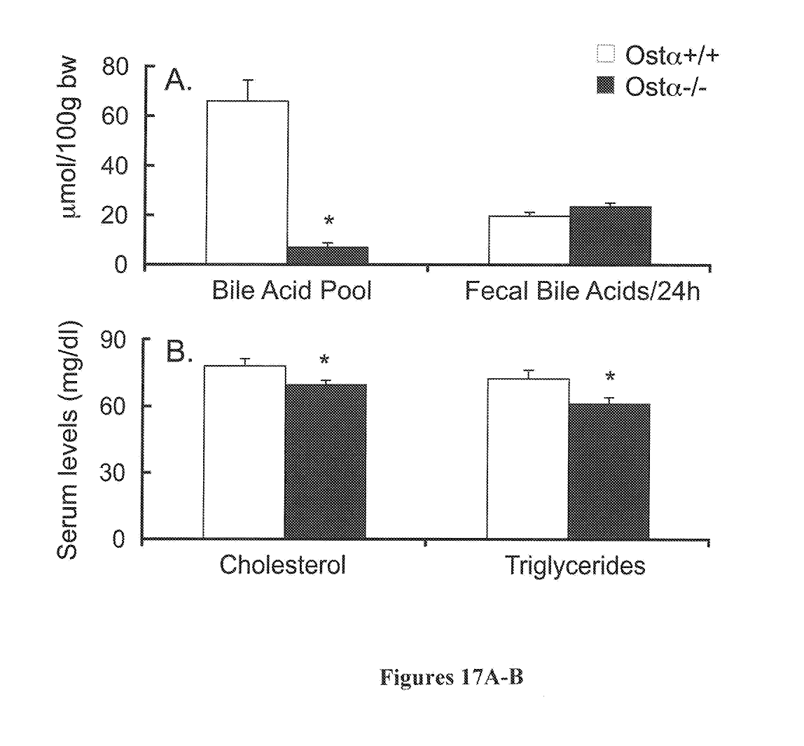

If Ostα-Ostβ is the critical intestinal basolateral bile acid transporter, one would predict that Ostα^{-/-} mice should have higher fecal bile acid excretion rates, and lower tissue bile acid levels. Because the vast majority of the bile acids are present in the liver, gallbladder, bile duct, and small intestine, these tissues were combined to assess the total bile acid pool size. The results reveal that the bile acid pool size in Ostα^{-/-} mice was approximately 90% smaller (FIG. 17A) and serum levels were about 60% lower when compared to wild type mice (Table 3, below). However, fecal bile acid excretion rates were similar (FIG. 17A), indicating a defect in intestinal bile acid absorption.

TABLE 3
Serum Clinical Chemistry Profile of Ostα^{+/+} and Ostα^{-/-} Mice

| | Ostα^{+/+} | Ostα^{-/-} |
|---|---|---|
| Bile Acids (μmol/l) | 7.2 ± 1.0 | 2.8 ± 0.3* |
| Total Bilirubin (mg/dl) | 0.4 ± 0.1 | 0.3 ± 0.1 |
| Glucose (mg/dl) | 205 ± 15 | 248 ± 13* |
| Phosphorus (mg/dl) | 8.7 ± 0.6 | 8.7 ± 0.5 |
| Total Protein (g/dl) | 5.0 ± 0.2 | 4.9 ± 0.2 |
| Calcium (mg/dl) | 8.8 ± 0.2 | 9.0 ± 0.1 |
| Blood Urea Nitrogen (mg/dl) | 31 ± 2 | 27 ± 1 |
| Creatinine (mg/dl) | 0.19 ± 0.03 | 0.20 ± 0.01 |
| Albumin (g/dl) | 2.8 ± 0.1 | 2.8 ± 0.1 |
| Alanine Aminotransferase (U/l) | 85 ± 26 | 90 ± 23 |
| Aspartate Aminotransferase (U/l) | 462 ± 107 | 590 ± 128 |
| Alkaline Phosphatase (U/l) | 158 + 17 | 149 + 10 |

Values are means ± SE, n = 8 female mice for each genotype. The animals were starved overnight.
*Statistically significant from Ostα^{+/+} mice, p < 0.05.

### Example 14

### Decreased Deal Absorption of [³H]Taurocholate and [³H]Estrone 3-Sulfate in Ostα^{-/-} Mice

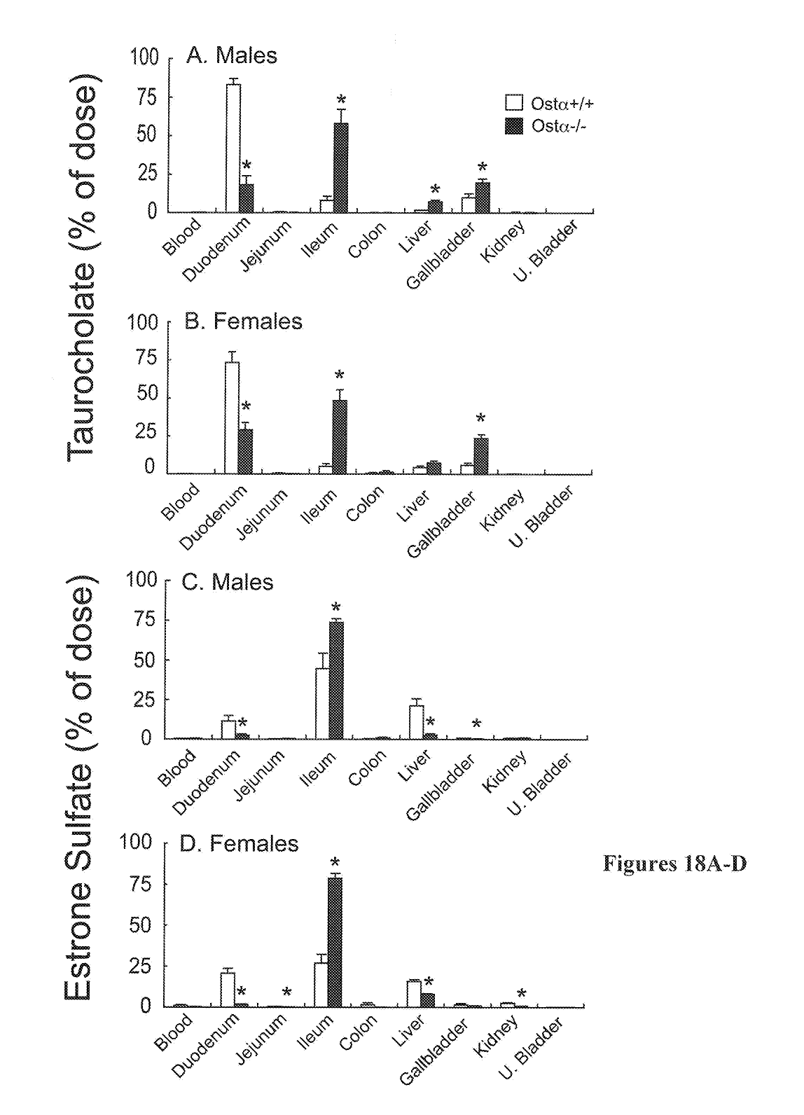

To examine the role of Ostα-Ostβ in the ileal absorption of a bile acid and a steroid conjugate, [³H]taurocholic acid and [³H]estrone 3-sulfate were injected directly into the ileal lumen of anesthetized male and female Ostα^{+/+} and Ostα^{-/-} mice, and tissue distribution of radioactivity measured at short time points thereafter (15 min for taurocholate and 30 min for estrone 3-sulfate). Ileal absorption of both compounds was lower in Ostα^{-/-} mice (FIG. 18), indicating that Ostα-Ostβ is important for this process. In particular, the amount of taurocholate absorbed from the ileum of Ostα^{-/- mice was} 60-80% lower than in the wild type animals (FIGS. 18A and B). Of the taurocholate that was absorbed by the Ostα^{-/-} mice, a significant fraction was found in the liver and gallbladder at the 15 min time point (FIGS. 18A and B), whereas in the wild type animals most of the taurocholate had already reached the duodenum by this time point. Estrone sulfate was absorbed more slowly than taurocholate by both wild type and Ostα^{-/- mice; only about} 60-70% of the estrone sulfate was removed from the ileum of wild type animals in 30 min, versus >90% absorption for taurocholate in only 15 min (FIG. 18). Estrone sulfate absorption was markedly lower in the Ostα^{-/-} mice when compared to the wild type mice (FIGS. 18C and D). Given that only a small fraction of the estrone sulfate was absorbed from the ileum of Ostα^{-/-} mice (i.e., 20-30%), relatively little radioactivity was recovered in the tissues from these animals (FIGS. 18C and D).

Note, however, that these data also demonstrate that alternate or compensatory mechanisms are present in the Ostα^{-/-} mice that allowed taurocholate and estrone sulfate to still be absorbed, albeit less efficiently than in wild type animals.

### Example 15

### Altered Tissue Distribution [³H]Estrone 3-Sulfate and [³H]DHEAS in Ostα^{-/-} Mice

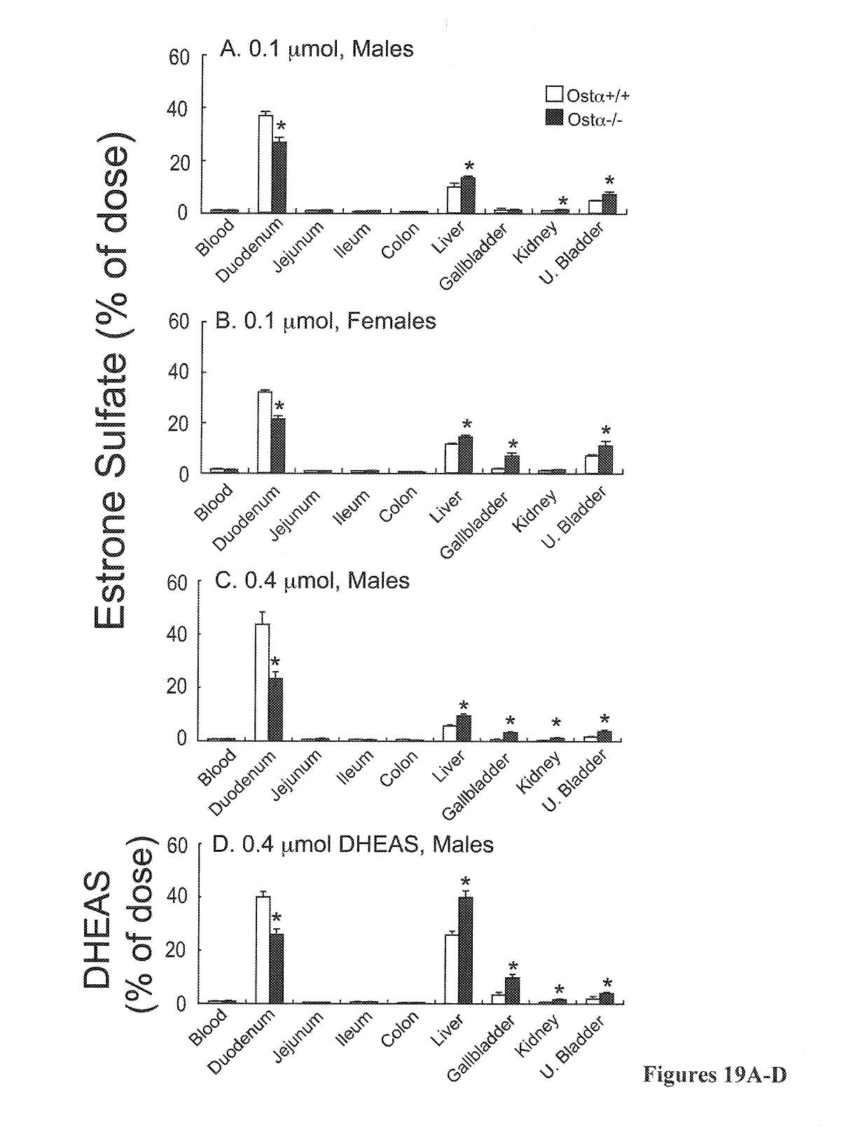

To characterize the role of Ostα-Ostβ in the in vivo disposition of known substrates for this transporter, the tissue distribution of [³H]estrone 3-sulfate and [³H]DHEAS was measured at 2 h after their intraperitoneal administration (FIG. 19). The Ostα^{-/-} mice had less radioactivity in their small intestine, and more in liver, kidney, and urinary bladder (FIG. 19), indicating altered disposition of these steroids and/or their metabolites. These observation support the hypothesis that Ostα-Ostβ is a major sterol transporter in the intestine, kidney and biliary epithelia (Ballatori et al., “Ostα-Ostβ, a Major Basolateral Bile Acid and Steroid Transporter in Human Intestinal, Renal, and Biliary Epithelia,” Hepatology 42: 1270-1279 (2005), which is hereby incorporated by reference in its entirety). In the kidney, the absence of Ostα-Ostβ is expected to diminish sterol reabsorption, and thus to increased urinary excretion of these compounds, and this hypothesis is supported by the data illustrated in FIG. 19. Although expression of Ostα-Ostβ is low in the whole mouse liver, these proteins are present in mouse cholangiocytes (Ballatori et al., “Ostα-Ostβ, a Major Basolateral Bile Acid and Steroid Transporter in Human Intestinal, Renal, and Biliary Epithelia,” Hepatology 42: 1270-1279 (2005), which is hereby incorporated by reference in its entirety), and thus this transporter may contribute to hepatobiliary disposition of bile acids and related molecules in the mouse (FIG. 19).

### Example 16

### Serum Cholesterol and Triglyceride Levels Are Also Lower in Ostα^{-/-} Mice

Because bile acids are required for intestinal lipid absorption, the diminished ability to absorb bile acids in Ostα^{-/-} mice may lead to a decrease in lipid absorption, and possibly to decreased serum lipid levels. Indeed, as illustrated in FIG. 17B, serum cholesterol and triglyceride levels were lower in the Ostα^{-/- mice (-}15% lower), whereas other serum parameters were generally similar (Table 3).

### Example 17

### Gene Expression Profiling in Ostα^{-/-} Intestine, Liver, and Kidney

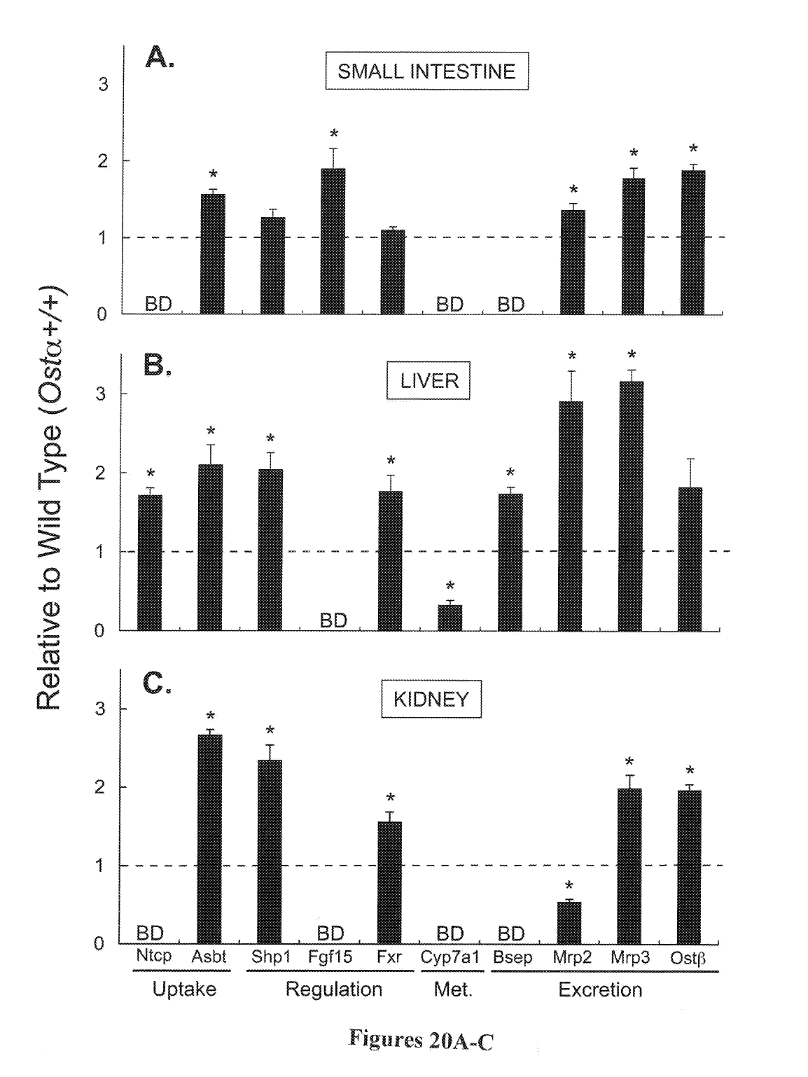

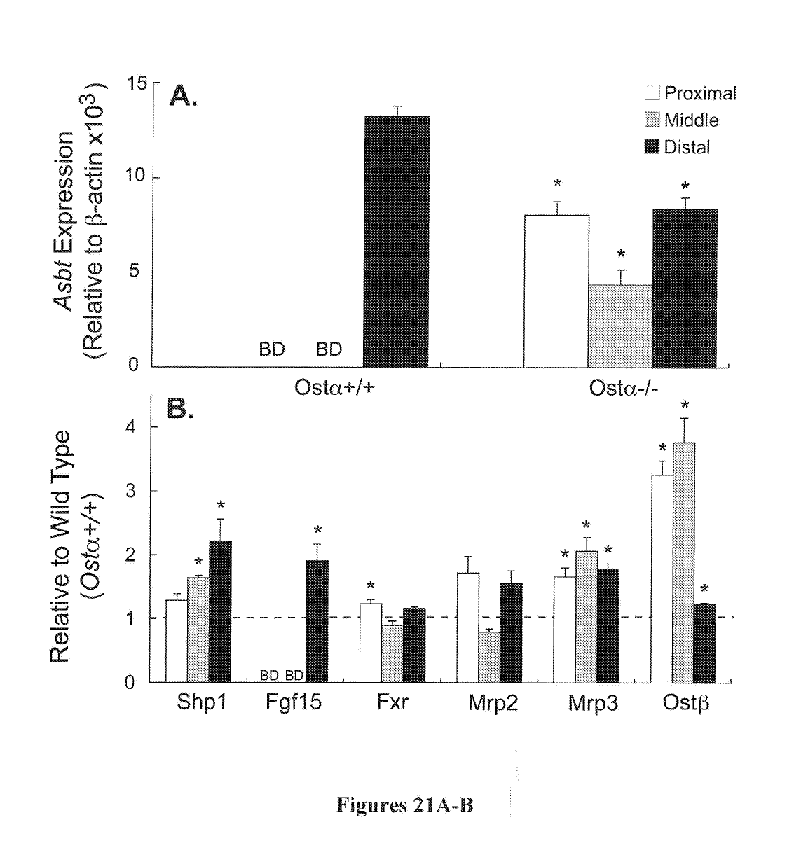

To further examine the functional significance of Ostα, and to assess whether expression of other putative bile acid and organic anion transporters is altered to compensate for the loss of Ostα-Ostβ in Ostα^{-/-} mice, real time RT-PCR was used to analyze transcript abundance of some of the key genes involved in bile acid homeostasis in the small intestine, kidney, and liver (FIGS. 20A-C). Because of the marked functional differences along the length of the small intestine, this tissue was divided into 3 equal sections, with the distal section representing mainly the ileum, and transcript abundance was measured in each section (FIGS. 21A-B).

If the proposed function of Ostα-Ostβ is correct, loss of this transporter should lead to the accumulation of bile acids in ileocytes, the major site of bile acid absorption, and thus to altered expression of bile acid-regulated genes. As expected, Asbt expression was decreased in the terminal portion of the small intestine (composed primarily of ileum), but was increased in proximal segments of the small intestine (FIG. 21A), as well as in liver and kidney (FIGS. 20B and C, respectively). Expression of Mrp3, a known alternative bile acid export pump, was upregulated in all three tissues analyzed (FIGS. 20A-C). In addition, Ntcp, Shp1, Fxr, Bsep and Mrp2 were increased in liver (FIG. 20B), and Shp1, Fxr, and Ostβ were increased in kidney (FIG. 20C), indicating altered bile acid homeostasis. Note that Mrp2 mRNA was higher in liver, but was lower in kidney (FIG. 20B-C), indicating differential tissue regulation of this organic anion export pump.

Of significance, these data also demonstrate that intestinal expression of fibroblast growth factor 15 (Fgf15), a key negative transcriptional regulator of the rate-limiting enzyme in bile acid synthesis (i.e., cholesterol 7α-hydroxylase, Cyp7a1), was increased (FIG. 20A), an effect that was seen mainly in the distal portion of the small intestine (FIG. 21B).

Because Fgf15 is strong a negative regulator of Cyp7a1 expression (Inagaki et al., “Fibroblast Growth Factor-15 Functions as an Enterohepatic Signal to Regulate Bile Acid Homeostasis,” Cell Metab. 2: 217-225 (2005); Jung et al., “Fxr Agonists and Fgf15 Reduce Fecal Bile Acid Excretion in a Mouse Model of Bile Acid Malabsorption,” J. Lipid Res. 48: 2693-2700 (2007); Kim et al., “Differential Regulation of Bile Acid Homeostasis by the Farnesoid X Receptor in Liver and Intestine,” J Lipid Res. 48: 2664-2672 (2007), which are hereby incorporated by reference in their entirety), one would predict that the increased levels of this growth factor may then decrease hepatic Cyp7a1, despite the smaller bile acid pool size in Ostα^{-/-} mice. Indeed, as illustrated in FIG. 20B, hepatic Cyp7a1 transcript abundance was markedly lower in Ostα^{-/-} mice. Overall, these changes in gene expression support the functional data described above, and provide insight into possible compensatory mechanisms.

### Discussion of Examples 12-17

The results presented in the preceding Examples demonstrate that Ostα-Ostβ is a major basolateral transporter of bile acids and structurally related molecules, that impairment of this transport step leads to a marked decrease in the bile acid pool size and serum bile acid levels, and is associated with lower serum cholesterol and triglyceride levels. These observations provide key insights into mechanisms of bile acid and sterol disposition, and have significant implications for human conditions related to imbalances in bile acid or lipid homeostasis.

Given the importance of bile acids to overall lipid balance, much effort has been devoted to the identification of mechanisms of bile acid transport, and to the development of strategies for altering bile acid disposition. The present observations in Ostα^{-/-} mice demonstrate that Ostα-Ostβ plays a key role in bile acid homeostasis, but also indicate that other transport mechanisms are able to compensate partially for the loss of this function.

The present findings also identify Ostα-Ostβ as a potential therapeutic target for interrupting the enterohepatic circulation of bile acids. In this regard, the one approach that has been used successfully in humans since the 1960s is the oral administration of non-absorbable bile acid-binding resins or polymers, which sequester the bile acids in the intestinal lumen and thus prevent their absorption (Bays et al., “Pharmacotherapy for Dyslipidaemia—Current Therapies and Future Agents,” Expert Opin. Pharmacother 4: 1901-1938 (2003), which is hereby incorporated by reference in its entirety). Other agents have also been evaluated as either inhibitors of intestinal bile acid absorption, including direct Asbt inhibitors, or as agonists/antagonists of the transcription factors that regulate bile acid levels, but none are currently available for human use (Bays et al., “Pharmacotherapy for Dyslipidaemia—Current Therapies and Future Agents,” Expert Opin. Pharmacother 4: 1901-1938 (2003), which is hereby incorporated by reference in its entirety). When intestinal bile acid uptake is interrupted with either bile acid sequestrants or with Asbt inhibitors, this leads to an increase in hepatic bile acid synthesis (Dawson et al., “Targeted Deletion of the Ileal Bile Acid Transporter Eliminates Enterohepatic Cycling of Bile Acids in Mice,” J. Biol. Chem. 278: 33920-33927 (2003); and Russell, D, “The Enzymes, Regulation, and Genetics of Bile Acid Synthesis,” Anna. Rev. Biochem. 72: 137-174 (2003), which are hereby incorporated by reference in their entirety), whereas the converse is seen in the Ostα^{-/-} mice, namely hepatic Cyp7a1 expression is decreased (FIG. 20B). As described in more detail below, these contrasting effects are most likely explained by the enhanced intestinal expression of Fgf15, an important negative regulator of hepatic bile acid synthesis (Inagaki et al., “Fibroblast Growth Factor-15 Functions as an Enterohepatic Signal to Regulate Bile Acid Homeostasis,” Cell Metab. 2: 217-225 (2005), which is hereby incorporated by reference in its entirety), when Ostα-Ostβ function is abrogated.

Bile acid concentrations are normally controlled by a feedback regulatory mechanism, whereby bile acid activation of Fxr represses hepatic transcription of Cyp7a1 levels, and thus leads to a decrease in bile acid synthesis. Bile acid activation of Fxr also leads to decreased expression of the bile acid uptake transporters Asbt and Ntcp, and to increased expression of the bile acid exporters Bsep and Ostα-Ostβ. Collectively, these transport proteins, along with the enzyme Cyp7a1, mediate a decrease in intracellular bile acid concentrations back to basal levels. Ileocytes, however, also express Fgf15, another key regulator of bile acid synthesis (Inagaki et al., “Fibroblast Growth Factor-15 Functions as an Enterohepatic Signal to Regulate Bile Acid Homeostasis,” Cell Metab. 2: 217-225 (2005), which is hereby incorporated by reference in its entirety). When intracellular bile acid levels in ileocytes are elevated (as they are expected to be in the Ostα^{-/-} mice), this leads to the Fxr-mediated induction of Fgf15 (Inagaki et al., “Fibroblast Growth Factor-15 Functions as an Enterohepatic Signal to Regulate Bile Acid Homeostasis,” Cell Metab. 2: 217-225 (2005), which is hereby incorporated by reference in its entirety). Fgf15 is then delivered to the liver, where it represses Cyp7a1 expression through a mechanism that involves Fgf receptor 4 (Fgfr4) and the orphan nuclear receptor Shp (Inagaki et al., “Fibroblast Growth Factor-15 Functions as an Enterohepatic Signal to Regulate Bile Acid Homeostasis,” Cell Metab. 2: 217-225 (2005), which is hereby incorporated by reference in its entirety). As illustrated in FIG. 20, intestinal expression of Fgf15 was higher and hepatic expression of Cyp7a1 was lower in Ostα^{-/-} mice, as predicted by this model.

These observations in Ostα^{-/-} mice contrast with those seen in Asbt^{-/-} mice (Dawson et al., “Targeted Deletion of the Ileal Bile Acid Transporter Eliminates Enterohepatic Cycling of Bile Acids in Mice,” J. Biol. Chem. 278: 33920-33927 (2003), which is hereby incorporated by reference in its entirety). The absence of Asbt leads to a diminished ability of the enterocytes to take up bile acids across their apical membranes, and thus, to relatively low intracellular bile acid levels. These low bile acid levels down-regulate Fgf15 expression, and thus relieve Fgf15-mediated repression of Cyp7a1 transcription. In addition, the low hepatocellular bile acid levels in Asbt^{-/-} mice also relieve the Fxr- and Shp-mediated repression of hepatic Cyp7a1 activity, with the net result being a 5-fold increase in Cyp7a1 expression and in bile acid synthesis rate in Asbt^{-/-} mice (Dawson et al., “Targeted Deletion of the Ileal Bile Acid Transporter Eliminates Enterohepatic Cycling of Bile Acids in Mice,” J. Biol. Chem. 278: 33920-33927 (2003), which is hereby incorporated by reference in its entirety).

Another significant difference between Ostα^{-/-} and Asbt^{-/-} mice relates to contrasting effects on serum cholesterol levels: serum total cholesterol is decreased in Ostα^{-/-} mice (FIG. 17B), but increased in Asbt^{-/-} mice (Dawson et al., “Targeted Deletion of the Ileal Bile Acid Transporter Eliminates Enterohepatic Cycling of Bile Acids in Mice,” J. Biol. Chem. 278: 33920-33927 (2003), which is hereby incorporated by reference in its entirety). The reason for this difference remains to be elucidated, but may also be related to altered bile acid and cholesterol homeostasis. In Asbt^{-/- mice, the increased Cyp}7a1-mediated cholesterol utilization leads to a decrease in hepatic cholesteryl ester levels (Dawson et al., “Targeted Deletion of the Ileal Bile Acid Transporter Eliminates Enterohepatic Cycling of Bile Acids in Mice,” J. Biol. Chem. 278: 33920-33927 (2003), which is hereby incorporated by reference in its entirety), which may stimulate either cholesterol synthesis or absorption, whereas the opposite is believed to be occurring in Ostα^{-/-} mice.

The preceding Examples also indicate that serum glucose levels are elevated in Ostα^{-/-} mice (Table 3). Although the mechanism for this increase is not clear, it may be related to the link between bile acid levels and glucose metabolism (Claudel et al., “The Farnesoid X Receptor: a Molecular Link Between Bile Acid and Lipid and Glucose Metabolism,” Arterioscler. Thromb. Vasc. Biol. 25: 2020-2030 (2005); and De Fabiani et al., “Coordinated Control of Cholesterol Catabolism to Bile Acids and of Gluconeogenesis Via a Novel Mechanism of Transcription Regulation Linked to the Fasted-To-Fed Cycle,”J. Biol. Chem. 278: 39124-39132 (2003), which are hereby incorporated by reference in their entirety). In particular, bile acids inhibit transcription of the gene encoding phosphoenolpyruvate carboxykinase (Claudel et al., “The Farnesoid X Receptor: a Molecular Link Between Bile Acid and Lipid and Glucose Metabolism,” Arterioscler. Thromb. Vasc. Biol. 25: 2020-2030 (2005); and De Fabiani et al., “Coordinated Control of Cholesterol Catabolism to Bile Acids and of Gluconeogenesis Via a Novel Mechanism of Transcription Regulation Linked to the Fasted-To-Fed Cycle,”J. Biol. Chem. 278: 39124-39132 (2003), which are hereby incorporated by reference in their entirety), the rate-limiting enzyme in gluconeogenesis, and thus, the low bile acid levels in Ostα^{-/-} mice may relieve this inhibition and result in higher glucose levels.

Although the present data indicate that Ostα-Ostβ is essential for sterol disposition, they also indicate that alternate absorptive mechanisms are able to partially compensate for the loss of this transporter, at least in adult mice. These compensatory mechanisms include intestinal hypertrophy (FIG. 16), and increased expression of alternate transport proteins (FIGS. 20 and 21). The increased intestinal length, and presumably increased surface area, would provide a longer transit time in the intestine, and thus would facilitate absorption by other mechanisms. The increased expression of Mrp3/Abcc3, an alternate basolateral bile acid export pump, and perhaps of other auxiliary transporters may subsume some of the sterol absorptive functions in the absence of Ostα-Ostβ.

In contrast to the adult mice, these compensatory gene expression changes may be inefficient in pre-weanling mice, and may thus explain the growth delay of the Ostα^{-/-} mice. Indeed, expression of Mrp3 and of some other Mrps is known to be very low in neonatal mice and reaches adult levels only after weaning (Maher et al., “Tissue Distribution and Hepatic and Renal Ontogeny of the Multidrug Resistance-Associated Protein (Mrp) Family in Mice,” Drug Metab. Dispos. 33: 947-955 (2005); and Tomer et al., “Differential Developmental Regulation of Rat Liver Canalicular Membrane Transporters Bsep and Mrp2,” Pediatr. Res. 53: 288-294 (2003), which are hereby incorporated by reference in their entirety). Likewise, expression of the nuclear receptors that are involved in bile acid and sterol homeostasis, including Fxr, are expressed at low levels in neonatal animals (Balasubramaniyan et al., “Multiple Mechanisms of Ontogenic Regulation of Nuclear Receptors During Rat Liver Development,” Am. J. Physiol. Gastrointest. Liver Physiol. 288: G251-G260 (2005), which is hereby incorporated by reference in its entirety). The low expression of these transcription factors and alternate sterol transporters in neonatal mice may account for the inability of Ostα-deficient mice to compensate for the lack of bile acid absorption; however, additional studies are needed to test these possibilities.

Taken together, the results of Examples 12-17 indicate that Ostα-Ostβ is a major basolateral transporter of bile acids and conjugated steroids in the intestine, kidney, and liver, and demonstrate that the resulting changes in bile acid levels have an effect on serum cholesterol, triglyceride, and glucose levels. In addition, these results indicate that targeted inhibition of Ostα-Ostβ may have advantages over other maneuvers that have been used to interrupt the enterohepatic circulation of bile acids.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

All of the features described herein (including any accompanying claims, abstract and drawings), and/or all of the steps of any method or process so disclosed, may be combined with any of the above aspects in any combination, except combinations where at least some of such features and/or steps are mutually exclusive.

1. A method of treating a patient having a condition or a disease associated with altered Ostα-Ostβ transporter activity comprising:
providing a therapeutic agent that inhibits Ostα-Ostβ heteromeric complex formation or Ostα-Ostβ transporter activity; and
administering the therapeutic agent to a patient having the condition or disease associated with altered Ostα-Ostβ transporter activity, wherein said administering is effective to treat the condition or disease associated with altered Ostα-Ostβ transporter activity.

2. The method according to claim 1, wherein the the disease or condition associated with altered Ostα-Ostβ transporter activity is selected from the group consisting of dyslipidemia, a disease associated with dyslipidemia, abnormal bile acid absorption, conditions associated with altered bile acid homeostasis, and abnormal lipid absorption.

3. The method according to claim 1, wherein the therapeutic agent inhibits Ostα-Ostβ heteromeric complex formation.

4-8. (canceled)

9. The method according to claim 3, wherein the therapeutic agent binds to an Ostβ transmembrane region.

10. The method according to claim 9, wherein the therapeutic agent that binds to the Ostβ transmembrane region is an antibody or aptamer that binds to at least a portion of the Ostβ transmembrane region comprising any one of the amino acid sequences selected from the group consisting of SEQ ID NOs:87-96.

11-12. (canceled)

13. The method according to claim 9, wherein the therapeutic agent that binds to an Ostβ transmembrane region is a peptide comprising any one of the sequences selected from the group consisting of SEQ ID NOs:17-86 or a fragment thereof.

14. The method according to claim 1, wherein the therapeutic agent inhibits Ostα-Ostβ transporter activity.

15. The method according to claim 14, wherein the therapeutic agent binds to the cysteine-rich region of Ostα located between transmembrane domains three and four.

16. The method according to claim 15, wherein the therapeutic agent is an antibody or aptamer that binds specifically to an epitope comprising the cysteine rich region of Ostα having an amino acid sequence of SEQ ID NO: 97, or an active fragment thereof.

17. (canceled)

18. The method according to claim 14, wherein the therapeutic agent binds to at least a portion of the amino-terminus of Ostβ.

19. The method according to claim 18, wherein the therapeutic agent is an antibody or aptamer that binds specifically to at least a portion of amino acids 1-27 of any one of the sequences selected from the group consisting of SEQ ID NOs: 9, 11, 13, and 15.

20. (canceled)

21. The method according to claim 2, wherein the disease or condition associated with altered Ostα-Ostβ transporter activity is dyslipidemia and is selected from the group consisting of hypercholesterolemia, hypertriglyceridemia, and hyperlipidemia.

22-63. (canceled)

64. The method according to claim 2, wherein the disease or condition associated with altered Ostα-Ostβ transporter activity is abnormal lipid absorption, and said lipid is selected from the group consisting of fats, oils, waxes, cholesterol, sterols, fat-soluble vitamins, monoglycerides, diglycerides, triglycerides, phospholipids

65. The method of claim 2, wherein the disease or condition associated with altered Ostα-Ostβ transporter activity is a condition associated with altered bile acid homeostasis and is selected from the group consisting of cholestasis, bile acid induced oxidative stress, necrotizing enterocolitis (NEC), and colon cancer.

66-85. (canceled)

86. A method of treating a patient having a condition or a disease associated with altered Ostα-Ostβ transporter activity comprising:
providing a therapeutic agent that enhances Ostα-Ostβ transporter activity or Ostα-Ostβ heteromeric complex formation; and
administering the therapeutic agent to a patient having the condition or disease associated with altered Ostα-Ostβ transporter activity, wherein said administering is effective to treat the condition or disease associated with altered Ostα-Ostβ transporter activity.

87. The method according to claim 86, wherein the therapeutic agent comprises an expression vector comprising one or more nucleic acid molecules encoding a functional Ostα-Ostβ transporter.

88. The method according to claim 87, wherein the one or more nucleic acid molecules comprise a nucleotide sequence selected from the group consisting of SEQ ID NOs: 2, 4, 6, 8, 10, 12, 14, and 16.

89. The method according to claim 87, wherein the expression vector comprises a tissue specific promoter operable in a tissue selected from the group consisting of liver, kidney, or small intestine.

90. The method according to claim 86, wherein said condition or disease associated with altered Ostα-Ostβ transporter activity is bile acid malabsorption or cholelithiasis.

91-94. (canceled)

95. An isolated nucleic acid or polypeptide that binds to either (i) an Ostα or an Ostβ transmembrane domain epitope, which upon binding prevents formation of an Ostα-Ostβ heteromeric complex, or (ii) an Ostα or Ostβ transport domain, which upon binding prevents transport by the Ostα-Ostβ heteromeric complex.

96. An isolated polypeptide according to claim 95, wherein the polypeptide is a peptide comprising any one of the amino acid sequences selected from the group consisting of SEQ ID NOs:17-97.

97. The isolated polypeptide of claim 95 further comprising a signal peptide sequence, wherein the signal peptide sequence is a cell uptake peptide, an endoplasmic reticulum signal peptide sequence, or a combination thereof.

98. (canceled)

99. The isolated polypeptide according to claim 95, wherein the polypeptide is an antibody or active fragment thereof that specifically binds to either an Ostα transmembrane domain epitope comprising at least a portion of any one of SEQ ID NOs: 17-86, an Ostβ transmembrane domain epitope comprising at least a portion of any one of SEQ ID NOs: 87-86, an OSTα transport domain epitope comprising SEQ ID NO: 97, or an OSTβ transport domain epitope comprising at least a portion of amino acids 1-27 of any one of the sequences selected from the group consisting of SEQ ID NOs: 9,11,13, and 15.

100-108. (canceled)

109. An isolated nucleic acid according to claim 95, wherein the nucleic acid is an aptamer that binds specifically to an Ostα transmembrane domain comprising at least a portion of any one of SEQ ID NOs: 17-86, an Ostβ transmembrane domain comprising at least a portion of any one of SEQ ID NOs: 87-96, an Ostα transport domain comprising SEQ ID NO:97, or an Ostβ transport domain comprising at least a portion of amino acids 1-27 of any one of the sequences selected from the group consisting of SEQ ID NOs: 9, 11, 13, and 15.

110. A pharmaceutical composition comprising the isolated nucleic acid or polypeptide according to claim 95 and a pharmaceutically acceptable carrier.

111. The pharmaceutical composition according to claim 110 further comprising a stabilizing reagent.

112. (canceled)

113. A non-human mammal comprising in its germ and somatic cells an artificially induced Ostα null mutation, wherein said mammal does not express Ostα protein.

114. The non-human mammal of claim 113, wherein the mammal is a rodent.

115. (canceled)
